(19) United States
(12) Patent Application Publication
(10) Pub. No.: US 2022/0105179 A1
(43) Pub. Date: Apr. 7, 2022

(54) GLYCOENGINEERING IMMUNOGLOBULIN E

(71) Applicant: The General Hospital Corporation, Boston, MA (US)

(72) Inventors: Robert M. Anthony, Cambridge, MA (US); Kai-Ting Chuang, Arlington, MA (US); Maya Kitaoka, Arlington, MA (US)

(21) Appl. No.: 17/430,949

(22) PCT Filed: Feb. 14, 2020
(86) PCT No.: PCT/US2020/018380

§ 371 Date: Aug. 13, 2021

Related U.S. Application Data
(60) Provisional application No. 62/808,456, filed on Feb. 21, 2019.
(60) Provisional application No. 62/808,449, filed on Feb. 21, 2019.

(51) Int. Cl. A61K 39/395; A61K 38/47; A61K 47/68; A61P 37/08; C12N 15/62; C12N 9/24; C12N 15/85; C12P 21/00

(52) U.S. Cl. CPC A61K 39/395; A61K 38/47; A61K 47/6815; A61P 37/08; C12N 2800/107; C12N 9/2402; C12Y 302/01018; C12N 15/85; C12P 21/005; C12N 15/62

(57) ABSTRACT

This disclosure relates to glycoengineering, and methods of utilizing glycoengineering for treating various diseases or disorders (e.g., IgE-mediated disorders). The methods include administering to the subject an effective amount of a composition comprising a fusion protein described herein. In some embodiments, the IgE-mediated disorder is an allergic disorder. In some embodiments, the allergic disorder is an anaphylactic allergy.

### CLAIM OF PRIORITY

This application claims the benefit of U.S. Provisional Patent Application Ser. No. 62/808,449, filed on Feb. 21, 2019, and 62/808,456, filed on Feb. 21, 2019. The entire contents of the foregoing are hereby incorporated by reference.

### FEDERALLY SPONSORED RESEARCH OR DEVELOPMENT

This invention was made with Government support under Grant Nos. AR068272 and AI139669 awarded by the National Institutes of Health (NIH). The Government has certain rights in the invention.

### TECHNICAL FIELD

This disclosure relates to glycoengineering immunoglobulin E (IgE), and methods of utilizing glycoengineering for treating various diseases or disorders. Also provided herein are methods for diagnosing allergies.

### BACKGROUND

Allergic disease is a global health burden affecting almost one in three individuals worldwide. Mechanistically, IgE antibodies bind to the surface of mast cells or basophils that express the IgE high affinity receptor, FcεRI³. Subsequent exposure to allergen crosslinks cell-bound IgE, leading to cellular activation and release of allergic mediators including histamine, prostaglandins, and leukotrienes³. This cascade culminates in the canonical symptoms of allergic disease, the most severe of which is anaphylaxis. While IgE that recognizes otherwise innocuous allergens is well established as the causative agent of most allergic diseases^{1,3}, testing for allergic disease remains relatively inaccurate⁴⁻⁶, and curative therapies, including oral immunotherapy, are cumbersome, and only partially effective⁸⁻¹⁰. Further, allergen-specific IgE is detected in many people who do not express allergic symptoms¹¹. Thus, while IgE is absolutely necessary for triggering the allergic cascade, it is not clear how IgE causes allergic disease in some circumstances and not others.

### SUMMARY

Approximately one-third of the world's population suffers from allergies^{1,2}. Allergen exposure crosslinks mast cell- and basophil-bound immunoglobulin E (IgE), triggering the release of inflammatory mediators, including histamine³. Although IgE is absolutely required for allergies, it is not understood why total and allergen-specific IgE concentrations do not reproducibly correlate with allergic disease⁴⁻⁶. It is well-established that glycosylation of IgG dictates its effector function and has disease-specific patterns. However, whether IgE glycans differ in disease states or impact biological activity is completely unknown. We therefore unbiasedly examined glycosylation patterns of total IgE from peanut-allergic and non-atopic individuals. This revealed an increase in sialic acid content on total IgE from peanut allergic individuals compared to non-atopic subjects. Sialic acid removal from IgE attenuated effector cell degranulation and anaphylaxis in multiple functional models of allergic disease. Therapeutic interventions, including sialic acid removal from cell-bound IgE with a FIERI targeted-neuraminidase, or administration of asialylated IgE, markedly reduced anaphylaxis. Together, these results reveal a role for IgE glycosylation, and specifically sialylation, in regulating allergy and anaphylaxis, and establish IgE sialylation as a biomarker and therapeutic target for allergies.

Thus, provided herein are fusion polypeptides comprising: an Immunolobulin E (IgE) or IgG antibody Fc domain region; and a sialidase or a functional portion thereof, preferably wherein the sialidase or a functional portion thereof can hydrolyze alpha-(2->3)-, alpha-(2->6)-, alpha-(2->8)-glycosidic linkages of terminal sialic residues on IgE. In some embodiments, the sialidase is NEU1, NEU2, NEU3, NEU4, or Vibrio cholerae serotype O1 sialidase. In some embodiments, the sialidase is a human sialidase. In some embodiments, the fusion polypeptide comprises an IgE CH2 region, an IgE CH3 region, and/or an IgE CH4 region; or an IgG CH2 and CH3 region.

Also provided herein are polynucleotides encoding the fusion polypeptides described herein, vectors comprising polynucleotides encoding the fusion polypeptides, and cells comprising the vectors, and optionally expressing the fusion polypeptides described herein.

Further, provided herein are methods for treating a subject having an IgE-mediated disorder. The methods include administering to the subject an effective amount of a composition comprising a fusion protein described herein. In some embodiments, the IgE-mediated disorder is an allergic disorder. In some embodiments, the allergic disorder is an anaphylactic allergy. In some embodiments, the allergic disorder is asthma, atopic dermatitis. allergic rhinitis, allergic conjunctivitis, eczema, or urticaria.

Additionally, provided herein are methods for preparing glycoengineered IgE, e.g., a composition comprising glycoengineered IgE, the method comprising: providing a composition comprising IgE, preferably human IgE, obtained from a plurality of subjects, contacting the IgE with a sialidase under conditions and for a time sufficient to remove sialylation, e.g., a desired amount of sialylation, from the IgE; thereby preparing glycoengineered IgE. In some embodiments, the method further comprises formulating the glycoengineered IgE for intravenous administration. In addition, provided herein are compositions comprising the glycoengineered IgE prepared by a method described herein, and a pharmaceutically acceptable carrier. In some embodiments, the compositions are formulated for intravenous administration.

Also provided herein are methods for treating a subject having an IgE-mediated disorder. The methods include administering to the subject an effective amount of a composition comprising glycoengineered IgE as described herein.

Further, provided herein are fusion proteins, glycoengineered IgE, and compositions comprising a fusion polypeptide and/or glycoengineered IgE as described herein, optionally with a pharmaceutically acceptable carrier, and the use of these compositions, fusion proteins, glycoengineered IgE and in treating a subject having an IgE-mediated disorder.

In some embodiments, the IgE-mediated disorder is an allergic disorder, e.g., an anaphylactic allergy. In some embodiments, the allergic disorder is asthma, atopic dermatitis. allergic rhinitis, allergic conjunctivitis, eczema, or urticaria.

This disclosure relates to glycoengineering, and methods of utilizing glycoengineering for treating various diseases or disorders (e.g., IgE-mediated disorders).

In another aspect, the disclosure relates to a fusion polypeptide having an antibody heavy chain CH2 region; an antibody heavy chain CH3 region; and a catalytic domain of sialidase, wherein the catalytic domain of sialidase removes sialic acid from a glycoprotein.

In some embodiments, the sialidase is NEU1, NEU2, NEU3, NEU4, or Vibrio cholerae serotype O1 sialidase. In some embodiments, the sialidase is a human sialidase.

In some embodiments, the fusion polypeptide has an IgG CH2 region, and an IgG CH3 region.

In some embodiments, the fusion polypeptide has an IgE CH2 region, an IgE CH3 region, and an IgE CH4 region.

In another aspect, the disclosure provides a polynucleotide encoding the fusion polypeptide as described herein.

In another aspect, the disclosure also relates to a vector having a polynucleotide sequence encoding the fusion polypeptide as described herein.

In one aspect, the disclosure relates to a cell having the vector as described herein, and the vector optionally expresses the fusion polypeptide as described herein.

In one aspect, the disclosure relates to a heteromultimer that has a first fusion polypeptide having an antibody heavy chain CH2 region, an antibody heavy chain CH3 region, and a catalytic domain of mannosidase, wherein the catalytic domain of mannosidase removes mannose from a glycoprotein; and a second fusion polypeptide having an antibody heavy chain CH2 region, an antibody heavy chain CH3 region, and a catalytic domain of sialidase, wherein the catalytic domain of sialidase removes sialic acid from a glycoprotein.

In some embodiments, the heteromultimer is a heterodimer, and the first fusion polypeptide associates with the second fusion polypeptide, thereby forming the heterodimer.

In some embodiments, the mannosidase is MAN1B1 or MAN2A1.

In some embodiments, the sialidase is NEU1, NEU2, NEU3, NEU4, or Vibrio cholerae serotype O1 sialidase.

In some embodiments, the first fusion polypeptide and the second polypeptide each has a human IgE CH2 region, a human IgE CH3 region, and a human IgE CH4 region.

In some embodiments, the first fusion polypeptide and the second polypeptide each has a human IgG CH2 region, and a human IgG CH3 region.

In another aspect, the disclosure also relates to methods of treating a subject having an IgE-mediated disorder. The methods involve administering to the subject an effective amount of a composition having the heteromultimer as described herein.

In some embodiments, the IgE-mediated disorder is an allergic disorder.

In some embodiments, the IgE-mediated disorder is an autoimmune disease.

In some embodiments, the IgE-mediated disorder is anaphylaxis.

In some embodiments, the allergic disorder is asthma. In some embodiments, the allergic disorder is atopic dermatitis. In some embodiments, the allergic disorder is allergic rhinitis, allergic conjunctivitis, eczema, or urticaria.

In one aspect, the disclosure relates to methods of treating a subject having an IgE-mediated disorder. The methods involve administering to the subject an effective amount of one or both of the following:

(a) a first polypeptide having a catalytic domain of mannosidase; and

(b) a second polypeptide having a catalytic domain of sialidase,

wherein the catalytic domain of the mannosidase removes mannose from a glycoprotein, and the catalytic domain of sialidase removes sialic acid from a glycoprotein.

In some embodiments, the first polypeptide further has a human IgE CH2 region, a human IgE CH3 region, and a human IgE CH4 region.

In some embodiments, the first polypeptide further has a human IgG CH2 region, and a human IgG CH3 region.

In some embodiments, the second polypeptide further has a human IgE CH2 region, a human IgE CH3 region, and a human IgE CH4 region.

In some embodiments, the second polypeptide further has a human IgG CH2 region, and a human IgG CH3 region.

In some embodiments, the IgE-mediated disorder is an allergic disorder. In some embodiments, the IgE-mediated disorder is an autoimmune disease. In some embodiments, the IgE-mediated disorder is anaphylaxis.

In some embodiments, the allergic disorder is asthma, atopic dermatitis, allergic rhinitis, allergic conjunctivitis, eczema, or urticaria.

In one aspect, the disclosure provides a heteromultimer that has a first fusion polypeptide having a collagen trimerizing domain and a catalytic domain of mannosidase; a second fusion polypeptide having a collagen trimerizing domain and a catalytic domain of sialidase; and a third fusion polypeptide having a collagen trimerizing domain, wherein the first fusion polypeptide, the second fusion polypeptide, and the third fusion polypeptide bind to each other, forming the heteromultimer.

In some embodiments, the third fusion polypeptide further has a catalytic domain of sialidase. In some embodiments, the third fusion polypeptide further has a catalytic domain of mannosidase.

In another aspect, the disclosure relates to a heteromultimer that has a tetramer having four streptavidin polypeptides; and four polypeptides, wherein each of the four polypeptides is linked with biotin, and one or more of the four polypeptides has a catalytic domain of mannosidase or a catalytic domain of sialidase, wherein each of the four polypeptides binds to the tetramer having the four streptavidin polypeptides.

In some embodiments, each of the four polypeptides has a catalytic domain of mannosidase or a catalytic domain of sialidase. In some embodiments, each of the four polypeptides has a catalytic domain of mannosidase. In some embodiments, each of the four polypeptides has a catalytic domain of sialidase.

In some embodiments, two of the four polypeptides each has a catalytic domain of mannosidase, and two of the four polypeptides each has a catalytic domain of sialidase.

In one aspect, the disclosure also relates to a heteromultimer that has an antibody or antibody fragment thereof; a catalytic domain of mannosidase; and/or a catalytic domain of sialidase, wherein the catalytic domain of mannosidase and the catalytic domain of sialidase each is linked to the antibody or antibody fragment thereof.

In some embodiments, the heteromultimer has an antibody, and the antibody has two antibody heavy chains, and two antibody light chains.

In some embodiments, the catalytic domain of mannosidase is linked to C-terminus of the antibody heavy chain. In some embodiments, the catalytic domain of mannosidase is linked to C-terminus of the antibody light chain.

In some embodiments, the catalytic domain of sialidase is linked to C-terminus of the antibody heavy chain. In some embodiments, the catalytic domain of sialidase is linked to C-terminus of the antibody light chain.

As used herein, the term “multimer” refers to a protein having two or more polypeptides or a polypeptide complex formed by two or more polypeptides. The polypeptides can associate with each other, forming a quaternary structure.

As used herein, the term “heteromultimer” refers to a multimer having more than one type of polypeptides.

As used herein, the term “homodimer” refers to a multimer having two identical polypeptides.

As used herein, the term “heterodimer” refers to a multimer having two polypeptides, and the two polypeptides are different.

As used herein, the term “luminal domain” or “enzymatic luminal domain” refers to the portion of a glycosylation enzyme that is located within the lumen of the Golgi apparatus in its native state. The enzymatic luminal domain of a glycosyltransferase is usually the soluble portion of the glycosylation enzyme.

As used herein, the term “soluble portion” or “soluble domain” refers to the portion of glycosylation enzyme that is soluble. For trans-Golgi glycosylation enzymes, the soluble portions are often the enzymatic luminal domains of the glycosylation enzymes. For non-trans-Golgi glycosylation enzymes, the entire glycosylation enzymes can be soluble. Thus, in some embodiments, the soluble portion can be the entire glycosylation enzyme or part of the glycosylation enzyme.

As used herein, the term “catalytic domain” refers to a portion of a protein that has a catalytic activity.

As used herein, the term “antibody-mediated disorder” refers to a disorder caused by or characterized by an increased level or an increased activity of an antibody.

As used herein, the term “IgE-mediated disorder” refers to a disorder caused by or characterized by an increased level or an increased activity of IgE.

As used herein, the term “linked” refers to being covalently or non-covalently associated, e.g., by a chemical bond (e.g., a peptide bond, or a carbon-carbon bond), by hydrophobic interaction, by Van der Waals interaction, and/or by electrostatic interaction.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-I. Glycan composition of IgE. A, A schematic of human IgE with N-linked glycosylation sites identified. Sites occupied by complex, biantennary glycans are noted by closed circles, oligomannose glycans by hatched circles, and unoccupied by X. Complex biantennary and oligomannose glycan schematics are shown at black and hatched circles, respectively; squares, GlcNAc; dark grey circles, mannose; triangle, fucose; light grey circles, galactose; grey diamonds, sialic acid. B, Total IgE titers from non-atopic (n=17) and peanut allergic (n=13) individuals; **P=0.0085. C, Allergen-specific IgE levels for Ara h 2 (peanut), Der p 1 (dust mite), Fel d 1 (cat), and Bet v 1 (birch pollen) from non-atopic (n=17) as compared to allergic (n=13) subjects; *P=0.0192. D, Allergen-specific IgE as a fraction (%) of total IgE across non-atopic (n=17) and allergic (n=13) individuals; **P=0.0086. E-I, Quantified glycan residues per IgE molecule on total IgE from non-atopic and peanut allergic individuals by glycopepetide mass spectrometry, showing total mannose (e, n=15 for non-atopic and 14 for allergic individuals, P=0.8467), fucose (f, n=10 for non-atopic and 11 for allergic individuals, P=0.0720), biGlcNAc (g, n=10 for non-atopic and 11 for allergic individuals, *P=0.0491), terminal galactose (h, n=14 for non-atopic and 19 for allergic individuals, ****P<0.0001), and terminal sialic acid (i, n=9 for non-atopic and 11 for allergic individuals, ***P=0.0009). Data plotted are mean±s.e.m., n.s., not significant and P values were determined by unpaired two tailed t test.

FIGS. 2A-B. Sialic acid and galactose distinguish allergic from non-atopic IgE. A, Receiver operating characteristic curve (ROC) for total number of variable IgE glycan moieties. ROC was performed for total IgE glycans isolated from allergic subjects as compared to non-atopic controls. Sialic acid (non-atopic n=9, allergic=11); Galactose (non-atopic n=14, allergic=19); Fucose (non-atopic n=14, allergic=15); biGlcNac (non-atopic n=14, allergic=19); Oligomannose (non-atopic n=15, allergic=14). B, Glycopeptide mass spectrometry analysis of site-specific N-glycan structures on total IgE from non-atopic (N) and allergic (A) individuals; Site 140 (non-atopic n=11, allergic n=11), Site 168 (non-atopic n=13, allergic n=15), Site 218 (non-atopic n=11, allergic n=17), Site 265 (non-atopic n=12, allergic n=19), Site 371 (non-atopic n=12, allergic n=15), Site 394 (non-atopic n=12, allergic n=11). The specific glycan structures per group are detailed in FIG. 7G. ****P<0.0001, ***P=0.0006. P values are determined by two-way ANOVA followed by Tukey's multiple comparison test.

FIGS. 3A-N. Sialic acid removal attenuates IgE. A, SNA lectin blot specific for α2,6-sialic acid and coomassie protein loading control of OVA-specific buffer-treated ^{Sia}mIgE and NEU-treated ^{As}mIgE. Images are representative of at least four independent digests. B, Quantification of ear blue coloration and representative ear images following OVA-induced PCA by PBS (n=2), OVA-specific ^{Sia}mIgE (n=8), OVA-specific ^{As}mIgE (n=12), OVA-specific ^{Re-Sia}mIgE (n=4). Data are representative of three experiments. ****P<0.0001; ns, P=0.9933 (one-way ANOVA with Tukey's multiple comparison test). C, Left, mean fluorescent intensity (MFI) and right, representative histograms of anti-mIgE on dermal mast cells following sensitization by PBS (n=5), OVA-specific ^{Sia}mIgE (n=6), or OVA-specific ^{As}mIgE (n=6) in mouse ears. ns, P=0.9017 (one-way ANOVA with Tukey's multiple comparison test). D, Binding of OVA-specific ^{Sia}mIgE and ^{As}mIgE to OVA as determined by ELISA. n=2 replicates and are representative of three experiments. ns, P>0.8040 for all concentrations tested (two-way ANOVA with Sidak's multiple comparisons test). E, F, Temperature change (E) and serum histamine quantified at defined intervals (F) following DNP-induced PSA in mice intravenously sensitized with PBS (n=3), DNP-specific ^{Sia}mIgE (n=5), or DNP-specific ^{As}mIgE (n=5). Data are representative of three experiments. ***P=0.0005, ****P<0.0001, ***P=0.0007 (two-way ANOVA with Tukey's multiple comparison test). G, Serum levels of DNP-specific ^{Sia}mIgE (n=4), or DNP-specific ^{As}mIgE (n=5) at defined intervals after intraperitoneal systemic administration as determined by ELISA. Data are representative of three experiments. ns, P>0.7948 for all time points (two-way ANOVA with Sidak's multiple comparisons test). H, Temperature change following PFA elicited by oral administration of TNP-OVA in mice sensitized with PBS (n=2), TNP-specific ^{Sia}mIgE (n=4), or TNP-specific ^{As}mIgE (n=4). Data are representative of three experiments. **P=0.0057 and 0.0035, ****P<0.0001, ***P=0.0005 (two-way ANOVA with Tukey's multiple comparison test). I, SNA lectin blot and coomassie loading control of OVA-specific buffer-treated ^{Sia}hIgE and NEU-treated ^{As}hIgE. Images are representative of at least four independent batches. J, OVA-induced β-hexosaminidase release by LAD2 mast cells sensitized with PBS, OVA-specific ^{Sia}hIgE or OVA-specific ^{As}hIgE. n=3 replicates and are representative of six experiments. ****P<0.0001; ns, P>0.9999 (two-way ANOVA with Tukey's multiple comparison test). K, β-hex degranulation after OVA stimulation of peripheral blood mononuclear cell-derived human mast cells sensitized with OVA-specific ^{Sia}hIgE and ^{As}hIgE. Mean and s.e.m. are plotted. ****P<0.0001; ns, P=0.9995 (two-way ANOVA with Sidak's multiple comparison test). L, Basophils expressing surface CD63 (left) and representative FACS plots following OVA stimulation on CD123⁺HLADR⁻ peripheral blood mononuclear cells sensitized with PBS (n=1), OVA-specific ^{Sia}hIgE (n=4) or OVA-specific ^{As}hIgE (n=4). Data are representative of four experiments. ****P<0.0001, **P=0.0017; ns, P=0.3829 (two-way ANOVA with Tukey's multiple comparison test). M, N, Binding kinetics of analytes OVA-specific ^{Sia}hIgE or OVA-specific ^{As}hIgE to ligands hFcεRIα (m) or OVA (n) loaded on biosensors. Analytes kinetics were performed with 3-fold serial dilution of analytes from 90 nM to 1 nM. Data are representative of three experiments. All data plotted are mean±s.e.m.

FIGS. 4A-I. Asialylated IgE modulation of anaphylaxis. A, Immunoblots of phosphorylated and total Syk and β-actin in LAD2 mast cells sensitized with PBS, OVA-specific SiahIgE or OVA-specific AshIgE after OVA stimulation for the indicated times. Images are representative of three independent experiments. B, OVA-induced Ca2+ flux traces showing fluo-4 fluorescence over fluorescence at time=0 (F/F0, left) and quantified maximal Ca2+ changes after OVA stimulation as measured by the difference between maximum (Fmax) over F0 (right) in Fluo-4 loaded LAD2 mast cells sensitized with PBS, OVA-specific ^{Sia}hIgE or OVA-specific ^{As}hIgE. Data are representative of three independent experiments. *P=0.0346 (two-tailed paired t-test). C, OVA-elicited degranulation in LAD2 mast cells sensitized with OVA-specific ^{Sia}hIgE and treated with ^{Sia}Fetuin or ^{As}Fetuin. n=3 replicates and are representative of three experiments. *P=0.0248, ****P<0.0001 (two-way ANOVA with Sidak's multiple comparison test). D, Quantification of ear blue coloration (left) and representative ear images (right) following OVA-induced PCA of mice sensitized with PBS (n=2), OVA-specific ^{Sia}mIgE (20 ng, n=6), both OVA-specific ^{Sia}mIgE (20 ng)+^{As}mIgE (200 ng) (n=3), or both OVA-specific ^{Sia}mIgE (20 ng)+mIgE isotype control (200 ng) (n=3). Data are representative of three experiments. *P=0.0478 and 0.0321; ns, P=0.9733 (one-way ANOVA with Tukey's multiple comparison test). E, Temperature change following DNP-induced PSA in mice receiving DNP-specific ^{Sia}mIgE on day 0 and PBS (n=6 for e), OVA-specific ^{Sia}mIgE (n=7 for e), or OVA-specific ^{As}mIgE (n=7 for e) on day 1. E, ***P=0.0001, *P=0.0211 and 0.0278. (two-way ANOVA with Tukey's multiple comparison test). F, Schematics of NEU^{Fcε}. Neuraminidase was linked to IgE Fc Cε2-4 by a peptide linker. G, OVA-induced β-hexosaminidase release by LAD2 mast cells sensitized with OVA-specific ^{Sia}hIgE and treated with PBS, NEU^{Fcε}, heat-inactivated NEU^{Fcε} (H-I NEU^{Fcε}) or IgE isotype control. n=3 replicates and are representative of three experiments. ****P<0.0001 (two-way ANOVA with Tukey's multiple comparison test). H, Peanut-induced β-hexosaminidase release by LAD2 mast cells sensitized with peanut allergic ^{Sia}hIgE treated with PBS, NEU^{Fcε}, or IgE isotype control. n=3 replicates and are representative of three experiments. ****P<0.0001 (two-way ANOVA with Tukey's multiple comparison test). I, Temperature change following OVA-induced PSA in mice receiving OVA-specific ^{Sia}mIgE on day 0 and PBS, NEU^{Fcε}, or IgE isotype control on day 1. n=4 per group and data are representative of three experiments. ***P=0.0008 and 0.0003, *P=0.0184 (two-way ANOVA with Tukey's multiple comparison test). All data plotted are mean±s.e.m.

FIGS. 5A-F. Functional aspects of allergen specific human IgE. A, Strategy for enriching IgE from human sera. B, Degranulation of human LAD2 mast cells sensitized with PBS, non-atopic or peanut allergic IgE stimulated by anti-human IgE and determined by β-hexosaminidase release. STATS C, Quantified MFI (left) and representative histograms (right) of anti-hIgE on human LAD2 mast cells sensitized with PBS, non-atopic, or allergic hIgE. STATS D, Binding of anti-hIgE from b to ^{Sia}hIgE and ^{As}hIgE as determined by ELISA shows no sialic acid dependent binding effects. n=2 replicates and are representative of three experiments. E, Specific glycans on IgE do not differ significantly between male and female subjects (n=9 males, n=12 females). F, Number of biGlcNAc residues differs significantly between 0-9 years old (n=2) and subjects of ages 10-19 (n=2, *P=0.0228), 20-29 (n=6, *P=0.0295) and 30-39 (n=7, *P=0.0019) respectively. Sialic acid, galactose and fucose do not differ across age groups. Data are presented as the mean±SEM; ns, not significant, *P<0.05, **P<0.01, ****P<0.0001 as determined by unpaired t test.

FIGS. 6A-E. Complex glycans observed on native human IgE. A, Representative MS/MS spectrum for N265 A2F glycopeptide showing B and Y ions from glycosidic bond cleavage as well as B ions from peptide bond cleavage. The Y1 ion used for quantification of glycopeptides is circled. B, Extracted ion chromatograms for IgE N265 sialylation variants from an allergic patient and non-allergic donor. C, Extracted ion chromatograms for IgE N168 sialylation variants from an allergic patient and non-allergic donor. D, Extracted ion chromatograms site specific N-glycosylation from chymotryptic digest of the IgE myeloma sample used as a standard. E, Extracted ion chromatograms site specific N-glycosylation from tryptic digest of the IgE myeloma sample used as a standard.

FIGS. 7A-G. Site-specific characterization of total IgE from peanut allergic and non-atopic individuals. A, Occupancy of N-linked glycosylation sites by glycans on non-atopic and allergic IgE; Site 140 (non-atopic n=15, allergic n=13), Site 168 (non-atopic n=16, allergic n=14), Site 218 (non-atopic n=15, allergic n=15), Site 265 (non-atopic n=12, allergic n=15), Site 371 (non-atopic n=15, allergic n=15), Site 383 (non-atopic n=16, allergic n=15), Site 394 (non-atopic n=13, allergic n=16). B, Configuration of oligomannose residues at site 394 does not differ between non-atopic (n=23) and allergic (n=18) groups. C, Total number of fucose residues per site of IgE, non-atopic, allergic; Site 140 (non-atopic n=15, allergic n=13), Site 168 (non-atopic n=15, allergic n=17), Site 218 (non-atopic n=15, allergic n=19), Site 265 (non-atopic n=12, allergic n=18), Site 371 (non-atopic n=15, allergic n=17). D, biGlcNAc residues by site of IgE, non-atopic compared to allergic; Site 140 (non-atopic n=15, allergic n=13), Site 168 (non-atopic n=16, allergic n=17), Site 218 (non-atopic n=15, allergic n=19), Site 265 (non-atopic n=16, allergic n=20), Site 371 (non-atopic n=16, allergic n=17). E, Total galactose residues from non-atopic and allergic subjects; Site 140 (non-atopic n=15, allergic n=14 STATS), Site 168 (non-atopic n=15, allergic n=17), Site 218 (non-atopic n=15, allergic n=19), Site 265 (non-atopic n=12, allergic n=19, **P=0.0014), Site 371 (non-atopic n=15, allergic n=17). F, Quantified sialic acid residues by IgE glycosylation site, non-atopic compared to allergic subjects; Site 140 (non-atopic n=14, allergic n=13), Site 168 (non-atopic n=15, allergic n=13, *P=0.0375), Site 218 (non-atopic n=15, allergic n=17), Site 265 (non-atopic n=12, allergic n=19, *P=0.0132), Site 371 (non-atopic n=15, allergic n=17). G, Representative structures for complex N-glycans. Data plotted are mean±s.e.m. P values are determined by two-way ANOVA followed by Sidak's multiple comparison test.

FIGS. 8A-C. IgE has α2,6-linked sialic acid. A, Protein gel stain and lectin blots of IVIG, native human IgE purified from allergic patients, and fetuin. Lectin SNA was used for α2,6- and MALI for α2,3-linked sialic acids detection. B, HPLC glycan traces of undigested or allergic human IgE or fetuin digested with sialidase from Arthrobacter ureafaciens for releasing α2,3-, α2,6-, α2,8- and α2,9-linked sialic acids or sialidase from Streptococcus pneumoniae for releasing α2,3-linked sialic acids. C, HPLC glycan traces of undigested or recombinant OVA-specific mIgE digested with sialidase from Arthrobacter ureafaciens for releasing α2,3-, α2,6-, α2,8- and 2,9-linked sialic acids.

FIGS. 9A-B PCA and dermal mast cell loading of ^{Sia}mIgE and ^{As}mIgE. A, Quantitation of vascular leakage by Evan's blue dye (left) and representative ear images from 3 mice (right) after PCA with PBS, or ^{Sia}mIgE and ^{As}mIgE specific for DNP. n=6 and are representative of three experiments. Mean and s.e.m. are plotted. **P=0.0047 (two-tailed unpaired t-test). B, Gating strategy for IgE loading on mouse skin ear mast cells. Representative FACS plots used to identify mast cells in mouse ears and determine IgE levels on mouse ear mast cells. SSC, side scatter.

FIGS. 10A-C. PSA reaction and serum levels of ^{Sia}mIgE and ^{As}mIgE after systemic sensitization. OVA-elicited anaphylaxis as measured by temperature drop in mice sensitized with PBS, OVA-specific ^{Sia}mIgE (n=4 for a and 6 for b) or ^{As}mIgE (n=5 for a and 6 for b) by intravenous (a) or intraperitoneal (b) injection. Data are representative of 3 independent experiments. Mean and s.e.m. are plotted. For A, ****P<0.0001 (two-way ANOVA with Tukey's multiple comparison test). For B, *P=0.0270 and 0.0122, **P=0.0012 and 0.0018, ****P<0.0001 (two-way ANOVA with Tukey's multiple comparison test). C, Serum levels of DNP-specific ^{Sia}mIgE and ^{As}mIgE in mice at defined time after systemically administration as determined by ELISA. n=4 for all group.

FIGS. 11A-C. FACS analysis of LAD2 mast cell loading of ^{Sia}hIgE and ^{As}hIgE, PBMC-derived mast cells, and primary basophils. A, MFI (left) and representative histogram (right) of surface-bound hIgE on LAD2 mast cells following sensitization with PBS, OVA-specific ^{Sia}hIgE or OVA-specific ^{As}hIgE. n=3 replicates and are representative of three experiments. ****P<0.0001, *P=0.0134 (one-way ANOVA with Tukey's multiple comparison test). B, Phenotypic staining by FACS of peripheral blood mononuclear cell-derived human mast cells. C, Gating strategy for basophil activation assay. Representative FACS plots used to determine basophil activation from PBMC.

FIG. 12.|OVA-specific PSA of OVA-specific SiamIgE, or OVA-specific AsmIgE isotype controls from FIG. 4E. Temperature change following OVA-induced PSA in mice receiving DNP-specific SiamIgE on day 0 and PBS, OVA-specific SiamIgE, or OVA-specific AsmIgE on day 1. n=4 for all groups. ****P<0.0001 (two-way ANOVA with Tukey's multiple comparison test). All data plotted are mean±s.e.m and are representative of three experiments.

FIGS. 13A-G. Characterization of NEU^{Fcε}. A, Protein gel stain (left) and immunoblot for mIgE (right) of native and denatured NEU^{Fcε}. B, Binding kinetics of analyte NEU^{Fcε} to ligand hFcεRIα on biosensor. Analytes kinetics were performed with 3-fold serial dilution of analyte from 24 to 0.3 nM. Data are representative of three experiments. C, MFI of surface-bound NEU^{Fcε} on LAD2 mast cells following overnight sensitization by FACS analysis. n=3 replicates and are representative of three experiments. D-G, Sialidase activity of NEU^{Fcε} determined by digestion of mIgE or fetuin overnight (D-F) and detection of protein loading by coomassie (D), terminal α2,6-sialic acid by SNA (E), and terminal galactose by ECL (F) or by the amount of substrate 2-O-(p-Nitrophenyl)-α-D-N-acetylneuraminic acid digested by NEU^{Fcε} in a colorimetric assay (G).

FIG. 14 lists the amino acid sequences of several exemplary glycosylation enzymes.

FIGS. 15A-B lists the amino acid sequences of exemplary fragment crystallizable region (Fc) of several human and mouse immunoglobulin E (IgE, FIG. 15A) and IgG (FIG. 15B).

FIG. 16 lists the amino acid sequences of several exemplary glycosylation enzyme-Fc fusion proteins.

FIG. 17 lists the amino acid sequences of exemplary dog glycosylation enzymes: Canine NEU1 (SEQ ID NO: 45); Canine NEU2 (SEQ ID NO: 46); Canine NEU3 (SEQ ID NO: 47).

FIG. 18 lists the amino acid sequences of exemplary cat glycosylation enzymes: Feline NEU1 (SEQ ID NO: 48); Feline NEU2 (SEQ ID NO: 49); Feline NEU3 (SEQ ID NO: 50); Feline NEU4 (SEQ ID NO: 51).

FIG. 19 lists the amino acid sequences of exemplary cow IgE and glycosylation enzymes: Bovine IgE heavy chain constant region (SEQ ID NO: 52); Bovine Sialidase-1 (NEU1)-lysosomal (SEQ ID NO: 52); Bovine Sialidase-3 (NEU3)-Plasma membrane (SEQ ID NO: 53).

FIG. 20 lists the amino acid sequences of exemplary horse IgE and glycosylation enzymes: Equine IgE heavy chain constant region (SEQ ID NO: 55); Equine Neuraminidase (NEU1)-lysosomal (SEQ ID NO: 56); Equine Neuraminidase (NEU2)-cytosolic (SEQ ID NO: 57); Equine Neuraminidase (NEU3)-membrane (SEQ ID NO: 58).

FIG. 21 lists an exemplary sequence encoding hNEU1 hIgEFc (SEQ ID NO: 59).

FIG. 22 lists an exemplary sequence encoding hNEU2 hIgEFc (codon optimized for mammalian expression) (SEQ ID NO: 60).

FIG. 23 lists an exemplary sequence encoding hNEU3 hIgEFc (SEQ ID NO:61).

FIG. 24 lists an exemplary sequence encoding hNEU4 hIgEFc (SEQ ID NO: 62).

FIG. 25 lists an exemplary sequence encoding hNEU1 mIgEFc (SEQ ID NO:63).

FIG. 26 lists an exemplary sequence encoding hNEU2 mIgEFc (Codon optimized for mammalian expression) (SEQ ID NO:64).

FIG. 27 lists an exemplary sequence encoding hNEU3 mIgEFc (SEQ ID NO:65).

FIG. 28 lists an exemplary sequence encoding hNEU4 mIgEFc (SEQ ID NO:66).

### DETAILED DESCRIPTION

IgE-mediated allergic diseases are multifactorial, with a broad range of clinical presentations. While the presence of peanut-specific IgE associates with peanut allergy, there is a high rate of false positive allergy test results^{4,6,9,35}. Many non-mutually exclusive mechanisms for this discrepancy exist, including differences in IgE affinity or epitope diversity for allergens, mast cell numbers, FcεRI expression levels, Syk signaling, allergen-specific IgG antibodies, anti-IgE antibodies, and regulatory T cells numbers^{36,37}. While IgE from primary allergic samples is severely limited because of its low serum concentrations, recent studies have identified and sequenced B cells that produce peanut-specific antibodies IgE^{9,38}. However, the role of post-translation modifications of the IgE constant chains, including glycosylation, in regulating allergic disease has not been considered. As demonstrated herein, sialic acid content on total IgE distinguishes peanut-allergic and non-atopic IgE. Further, IgE-mediated allergic reactions are attenuated through removal of sialic acid from IgE or administration of asialylated glycoproteins. The sialic acid content and its role in regulating IgE in other atopies and non-atopic conditions is not known³⁹⁻⁴¹. Glycoengineering has been applied to tailor therapeutic IgGs with desirable pro- and anti-inflammatory functions^{18,20}. The present studies revealed that modulating IgE sialic acid content can attenuate anaphylaxis and affirms the application of glycoengineering to allergic disease. Thus, the sialic acid content on IgE can be used as a biomarker for allergic disease, and modulating the IgE sialylation axis presents a powerful means to attenuate allergies and anaphylaxis.

The present disclosure shows engineered glycosylation enzymes can modulate antibody effector function by engineering antibody glycans in vivo for various therapeutic effects. As shown herein, sialic acid in IgE glycans are important for IgE functions; the present disclosure further provides engineered glycosylation enzymes for modulating IgE, e.g., by removing sialic acid (neuraminidase or sialidase) from IgE Fcs, and thus inhibiting IgE pro-allergic function or activity.

Thus, the present disclosure relates to methods and compositions comprising a fusion peptide comprising a catalytic domain of a deglycosylation enzyme (e.g., neuraminidase or sialidase) fused to Fc (e.g., IgG Fc or IgE Fc). The methods and compositions described herein can be used to modulate IgE effector function for various therapeutic effects.

### Glycans on IgE

The proteins and cells that make up the human body are decorated by sugars often referred to as glycans (Varki, A. Glycobiology 3, 97-130 (1993)). Glycans can be linked to many types of biological molecule to form glycoconjugates. The enzymatic process that links sugars/saccharides to themselves and to other molecules is known as glycosylation. Glycoproteins, proteoglycans, and glycolipids are the most abundant glycoconjugates found in mammalian cells.

Glycans have an important role in the function of many proteins. Glycans are saccharides (i.e., a plurality of monosaccharides linked glycosidically) that form the carbohydrate portion of glycoconjugates (e.g., glycoproteins, glycopeptides, peptidoglycans, glycolipids, glycosides and lipopolysaccharides). They can be added to proteins in the endoplasmic reticulum, and further modified as proteins travel through the Golgi apparatus. Precursor glycan structures can be attached to asparagine (N-linked), serine or threonine (O-linked), phospholipids (GPI), tryptophan (C-linked), or by phosphodiester bonds (phosphoglycosylation).

Immunoglobulin E (IgE) has two heavy chains (ε chain) and two light chains, with the E chain containing 4 Ig-like constant domains (Cε1, Cε2, Cε3, Cε4; also referred to as CH1, CH2, CH3, CH4). IgE antibodies are primary mediators of allergic disease, and are heavily glycosylated with 7 N-linked glyclosylation sites distributed across its four constant regions (Cε1-Cε4). The distinct glycans on IgE play important and divergent roles in allergic inflammation. Removal of the conserved oligomannose in the constant domains (e.g., Cε1, Cε2, Cε3, Cε4) prevents binding to the high affinity receptor FcεRI on FcεRI-expressing cells (e.g., mast cells and basophils), therefore can inhibit the function or activity of IgE.

Analysis of the glycosylation of human serum IgE indicated that oligomannose structures are present on IgE. In fact, IgE is the most heavily glycosylated monomeric immunoglobulin in mammals. There are six complex-type biantennary (N140, N168, N218, N265, N371, N383) and one oligomannose-type (N394) conserved N-linked glycosylation sites on the constant region of each heavy chain of IgE. The total glycan weight on E heavy chains contributes to ˜12% of the molecular weight of IgE.

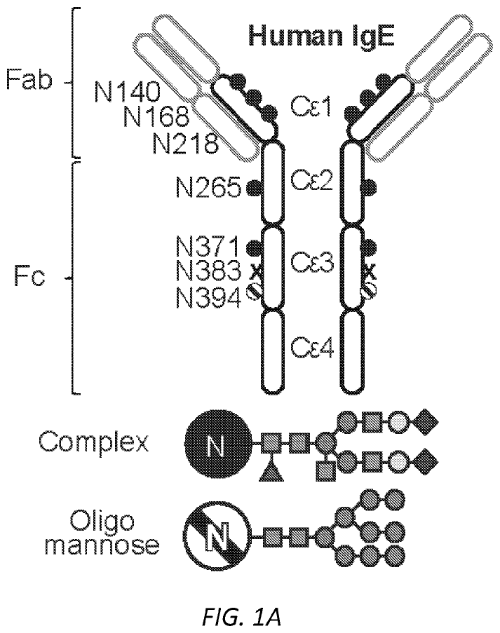

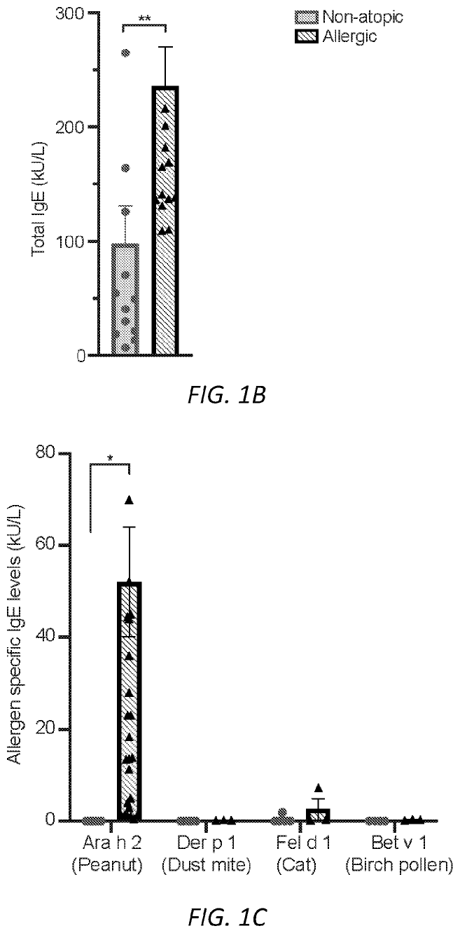

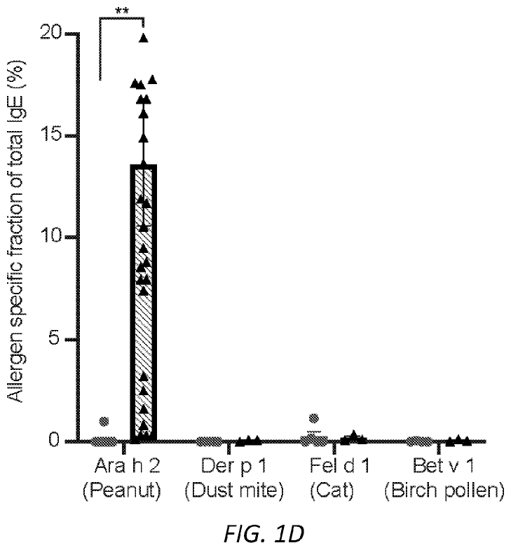

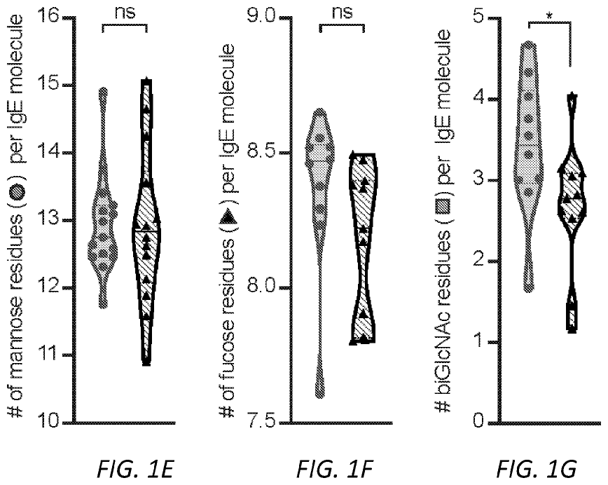

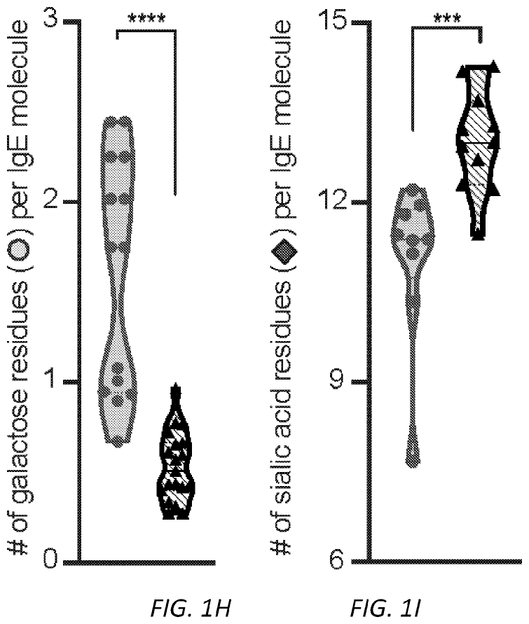

The composition of the single N-linked glycan on IgG antibodies profoundly influences its biological activity, and impacts the outcome of many diseases, including Dengue hemorrhagic fever¹², Mycobacterium tuberculosis latency¹³, Influenza vaccination¹⁴, Rheumatoid Arthritis^{7,15}, and Granulomatosis with polyangiitis^{16,17}. For example, IgG with afucosylated glycans gain affinity to the activating Fc receptor, FcγRIIIA, 50-fold, making IgG markedly more cytotoxic in vivo¹⁸. Conversely, terminal sialylation of the IgG glycan converts IgG into anti-inflammatory mediators, and is thought to be responsible for the immunomodulatory activity of high dose intravenous immunoglobulin^{19,20}. IgE is the most heavily glycosylated monomeric immunoglobulin with seven asparagine (N)-linked glycosylation sites distributed across the heavy chains of human IgE (hIgE)^{7,21}. However, whether particular IgE glycans are associated with allergic disease, or impact IgE function, is completely unknown. IgE is the least abundant antibody class in circulation, and, as such, analysis of hIgE glycosylation has been restricted to samples from subjects with myelomas, hyper IgE syndromes, hyperimmune syndromes pooled from multiple donors, or recombinant IgE²¹⁻²⁴. These studies revealed a single N-linked oligomannose glycan at N394 on IgE, N383 is unoccupied, and the remaining five sites are occupied by complex antennary glycans (FIG. 1a)²⁵. Previously, we and others demonstrated the oligomannose at N394 was required for appropriate IgE folding and FcεRI binding^{23,26} to initiate effector functions.

IgE Fc glycans can be removed by enzymatic treatment with mannosidase, neuraminidase, Endo F, and/or PNGase F. The enzymatic treatment can inhibit binding of IgE molecules or IgE-Fc fragments to FcεRI. Mutagenesis of the conserved N394 site, which corresponds to N297 on IgG Fc, also reduces the binding to FcεRI.

A detailed description regarding glycans on IgE and the functions thereof can be found, e.g., in Arnold, et al., “The glycosylation of human serum IgD and IgE and the accessibility of identified oligomannose structures for interaction with mannan-binding lectin.” The Journal of Immunology 173.11 (2004): 6831-6840; Shade, et al., “A single glycan on IgE is indispensable for initiation of anaphylaxis.” Journal of Experimental Medicine 212.4 (2015): 457-467; Shade, et al., “Antibody glycosylation and inflammation.” Antibodies 2.3 (2013): 392-414; and Plomp, et al., “Site-specific N-glycosylation analysis of human immunoglobulin E.” Journal of proteome research 13.2 (2013): 536-546; each of which is incorporated herein by reference in its entirety.

### Glycosylation Enzymes

Glycosylation enzymes are responsible for the reaction in which a carbohydrate, i.e. a glycosyl donor, is attached to a hydroxyl or other functional group of another molecule (a glycosyl acceptor, e.g., proteins, lipids, and glycans). There are many different kinds of glycosylation enzymes, e.g., α-2,6 sialyltransferase (ST6GAL1), β-1,4-galactosyltransferase 1 (B4GALT1), mannosyl-oligosaccharide 1,2-alpha-mannosidase (MAN1B1), alpha-mannosidase 2 (MAN2A1), human sialidase-1 (NEU1), human sialidase-2 (NEU2), human sialidase-3 (NEU3), human sialidase-4 (NEU4), Vibrio cholerae serotype O1 sialidase, Elizabethkingia meningoseptica Endo F1, endo-beta-N-acetylglucosaminidase (Endo S), etc. As shown herein, sialic acid removal attenuated IgE effector functions. Thus, provided herein are fusion proteins in which soluble portions (or the enzymatic luminal domains) or the catalytic domains of sialidases can be fused with Fc (e.g., IgG Fc or IgE Fc), or other appropriate peptides to form multimers, and can be used in any methods described herein.

### Sialidase

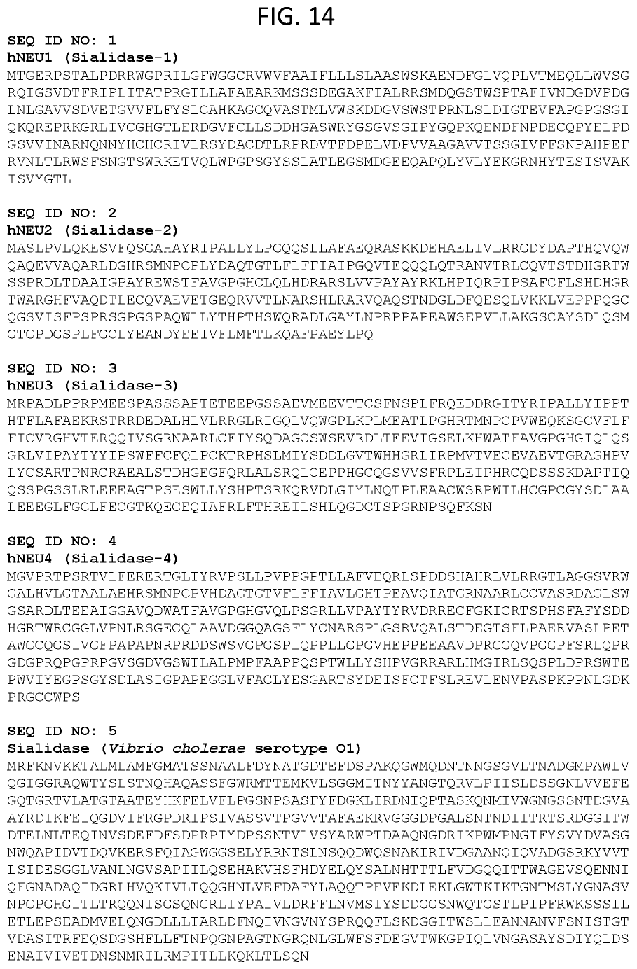

Sialidases (also known as neuraminidases) hydrolyze alpha-(2->3)-, alpha-(2->6)-, alpha-(2->8)-glycosidic linkages of terminal sialic residues in oligosaccharides, glycoproteins, glycolipids, colominic acid and synthetic substrates. There are four types of human sialidases. They are classified according to their major intracellular location as intralysomal (NEU1), cytosolic (NEU2), plasma membrane (NEU3) and lysosomal or mitochondrial membrane (NEU4) associated sialidases. These human isoforms are distinct from each other in their enzymatic properties as well as their substrate specificity. The sequences for NEU1 (SEQ ID NO: 1), NEU2 (SEQ ID NO: 2), NEU3 (SEQ ID NO: 3) and NEU4 (SEQ ID NO: 4) are shown in FIG. 14. A detailed description of human sialidases and their functions can be found, e.g., in Magesh, et al. “Homology modeling of human sialidase enzymes NEU1, NEU3 and NEU4 based on the crystal structure of NEU2: hints for the design of selective NEU3 inhibitors.” Journal of Molecular Graphics and Modelling 25.2 (2006): 196-207, which is incorporated by reference in its entirety.

Sialidases can also be found in bacteria, e.g., Vibrio cholerae. Vibrio cholerae is a Gram-negative, comma-shaped bacterium. Some strains of V. cholerae can cause cholera. Vibrio cholerae serotype O1 sialidase has been suggested to be a pathogenic factor in microbial infections. It facilitates cholera toxin binding to host intestinal epithelial cells by converting cell surface polysialogangliosides to GM1 monogangliosides. The sequence for Vibrio cholerae serotype O1 sialidase is shown in FIG. 14 (SEQ ID NO: 5). The function and the properties of Vibrio cholerae serotype O1 sialidase are known in the art, and are described, e.g., in Jermyn, William S., and E. Fidelma Boyd. “Characterization of a novel Vibrio pathogenicity island (VPI-2) encoding neuraminidase (nanH) among toxigenic Vibrio cholerae isolates.” Microbiology 148.11 (2002): 3681-3693; and Xiao, Han, et al. “Precision glycocalyx editing as a strategy for cancer immunotherapy.” Proceedings of the National Academy of Sciences (2016): 201608069; each of which is incorporated herein by reference in its entirety.

Thus, exemplary neuraminidases useful in the methods and compositions described herein include human NEU1, NEU2, NEU3, and NEU4; and Vibrio cholerae serotype O1 sialidase. See, e.g., FIG. 14.

NEU1 can include, e.g., human NEU1, e.g., the full length soluble NEU1 (SEQ ID NO: 1) or an active portion thereof comprising the luminal domain of human NEU1 (amino acids: 48-415 of SEQ ID NO: 1) and/or the catalytic domain residues of human NEU1 (including catalytic amino acid residues: R78, R97, D103, D135, S156, E264, R280, Q282, R342, Y370, and E394 of SEQ ID NO: 1).

NEU2 can include, e.g., human NEU2, e.g., the full length, soluble NEU2 (SEQ ID NO: 2) or an active portion thereof comprising the active site residues of human NEU2 (amino acids: R21, D46, M85, E111, Y179, Y181, L217, R237, R283, S288, and Y377 of SEQ ID NO: 2).

NEU3 can include, e.g., human NEU3, e.g., the full length human NEU3 (SEQ ID NO: 3) or an active portion thereof comprising the putative catalytic active sites of human NEU3 (amino acids: R25, R45, D50, M87, N88, R108, Q115, A160, E225, R235, R340, Y370, and E387 of SEQ ID NO: 3).

NEU4 can include, e.g., human NEU4, e.g., the full length NEU4 (SEQ ID NO: 4) or an active portion thereof comprising the catalytic active sites of human NEU4 (amino acids: R35, R55, D59, N88, V117, E234, R254, P256, R381, Y431, and E452 of SEQ ID NO: 4).

Vibrio cholerae serotype O1 sialidase can include, e.g., the full length sialidase (SEQ ID NO: 5) or an active portion thereof comprising the catalytic active sites of sialidase (AA25-781, as the first 24AA correspond to the signal peptide).

The active portions retain the ability of the full-length proteins to hydrolyze alpha-(2->3)-, alpha-(2->6)-, alpha-(2->8)-glycosidic linkages of terminal sialic residues on IgE.

The enzymes, the soluble portions thereof (or the luminal domains), the catalytic domains thereof, active sites, and catalytic amino acid residues of these glycosylation enzymes are described, e.g., in Seyrantepe, Volkan, et al. “Neu4, a novel human lysosomal lumen sialidase, confers normal phenotype to sialidosis and galactosialidosis cells.” Journal of Biological Chemistry 279.35 (2004): 37021-37029; Chavas, Leonard M G, et al. “Crystal Structure of the Human Cytosolic Sialidase Neu2—Evidence For The Dynamic Nature Of Substrate Recognition.” Journal of Biological Chemistry 280.1 (2005): 469-475; MONTI, Eugenio, et al. “Identification and expression of NEU3, a novel human sialidase associated to the plasma membrane.” Biochemical Journal 349.1 (2000): 343-351; and Seyrantepe, Volkan, et al. “Molecular pathology of NEU1 gene in sialidosis.” Human mutation 22.5 (2003): 343-352; each of which is incorporated by reference herein in its entirety.

In some embodiments, the sialidase used in the present methods is not receptor destroying enzyme (RDE) (II). Yamazaki et al., J Biol Chem. 2019 Apr. 26; 294(17):6659-6669. Epub 2019 Mar. 4.

### Nucleic Acid Sequences and Amino Acid Sequences

This disclosure provides various nucleic acid sequences and amino acid sequences.

In some embodiments, the nucleic acid sequence is at least 60%, 65%, 70%, 75%, 80%, 85%, 90%, 91%, 92%, 93%, 94%, 95%, 96%, 97%, 98%, 99% identical to any of the nucleic acid sequences disclosed herein. In some embodiments, the nucleic acid sequence is identical to any of the sequences described in this disclosure.

In some embodiments, the amino acid sequence is at least 60%, 65%, 70%, 75%, 80%, 85%, 90%, 91%, 92%, 93%, 94%, 95%, 96%, 97%, 98%, 99% identical to any of the amino acid sequences disclosed herein. In some embodiments, the amino acid sequence is identical to any of the sequences described in this disclosure.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

### Neu-IgE Fc Fusion Proteins

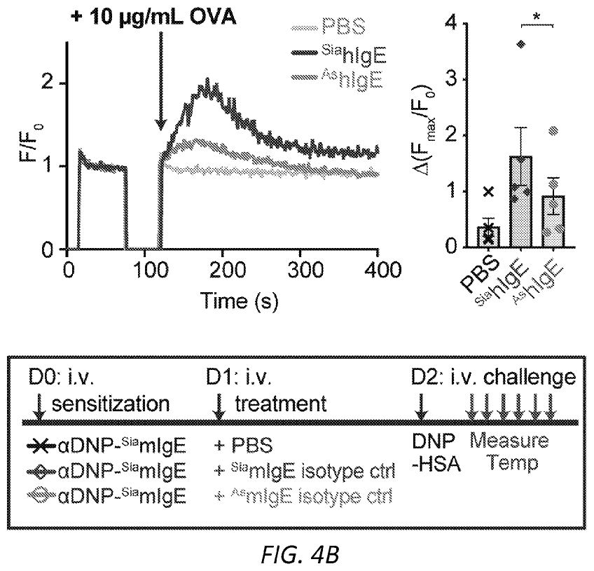

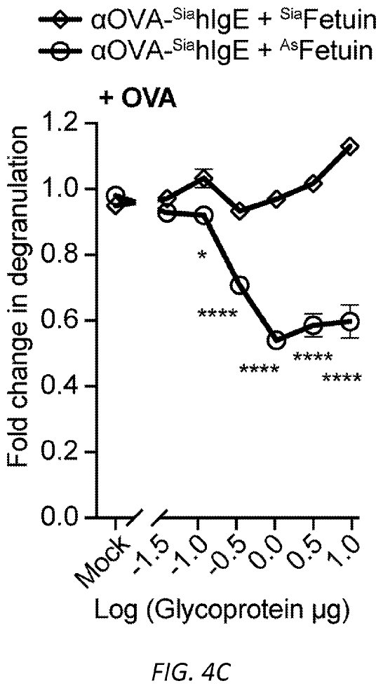

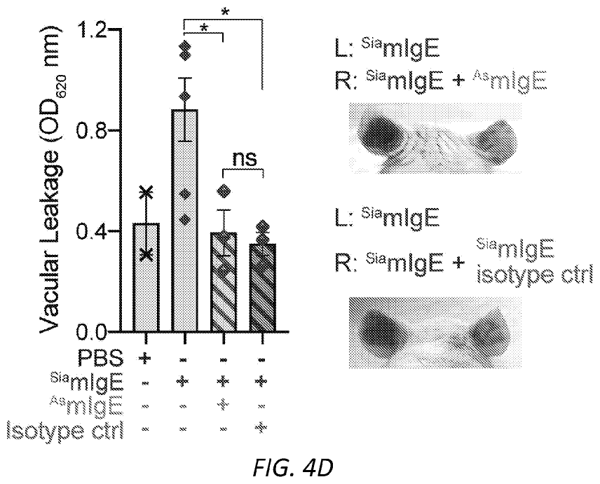

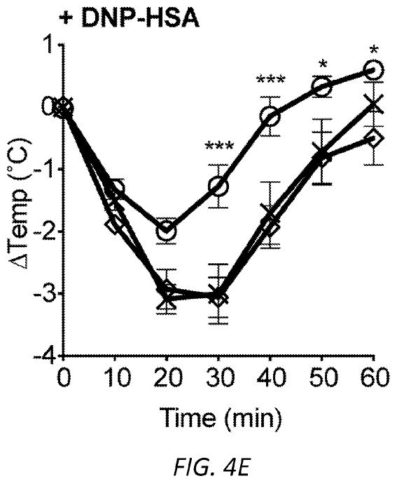

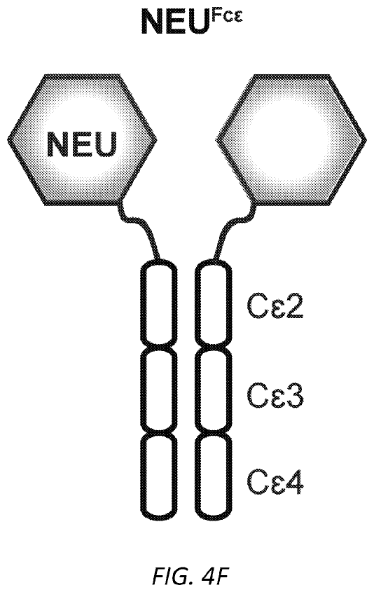

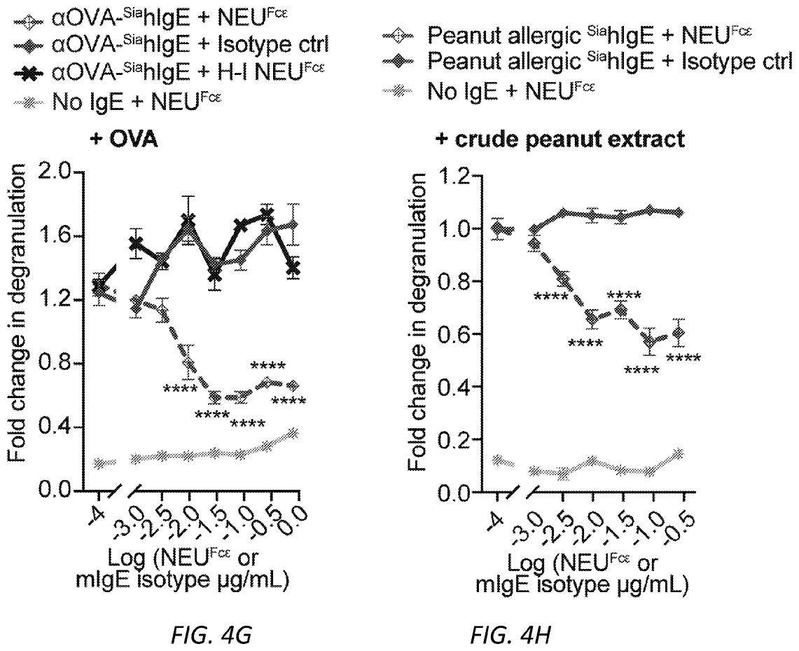

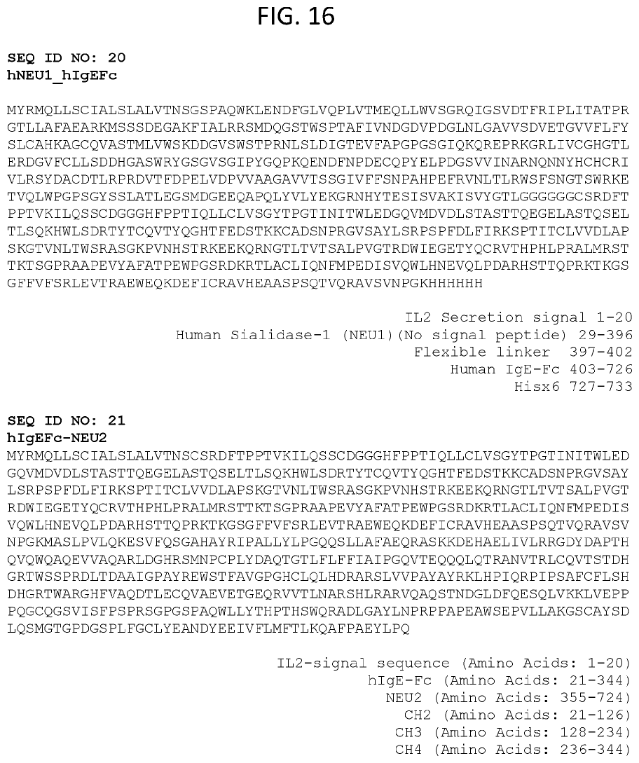

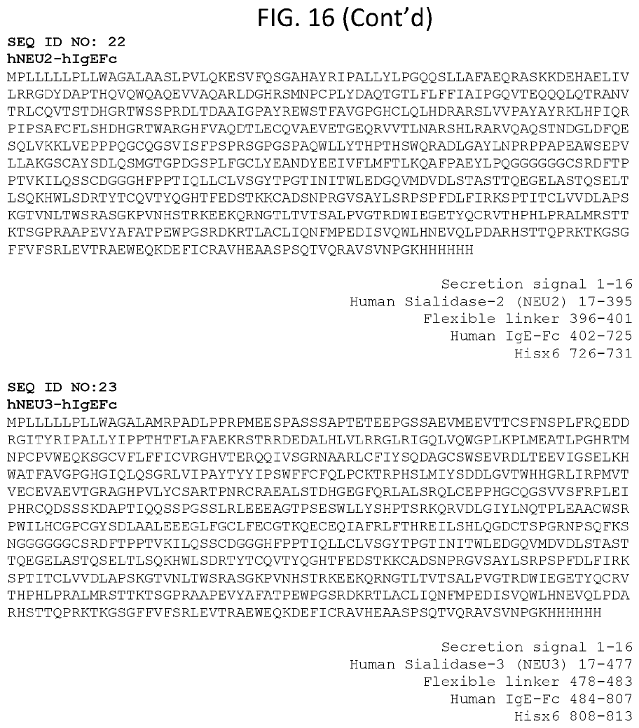

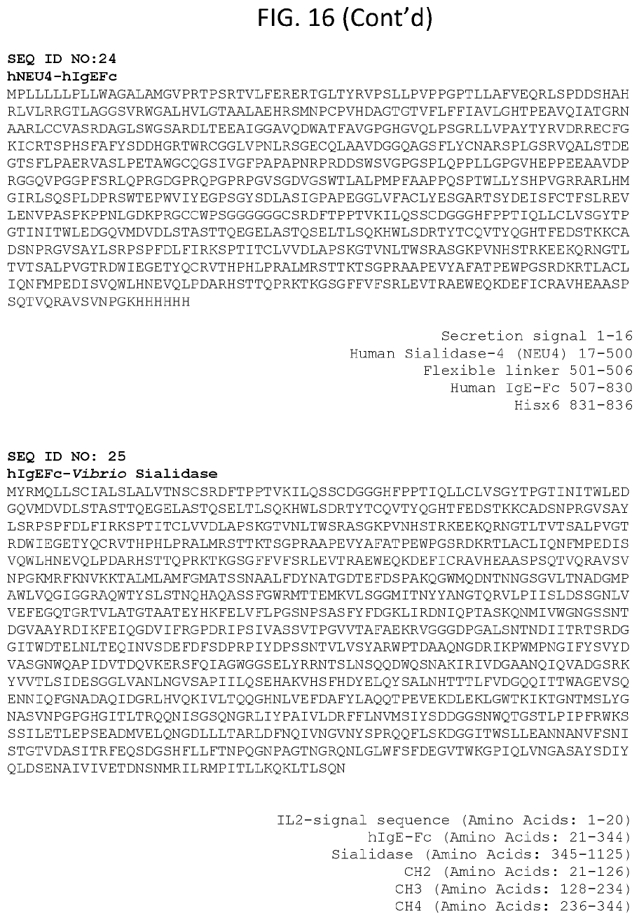

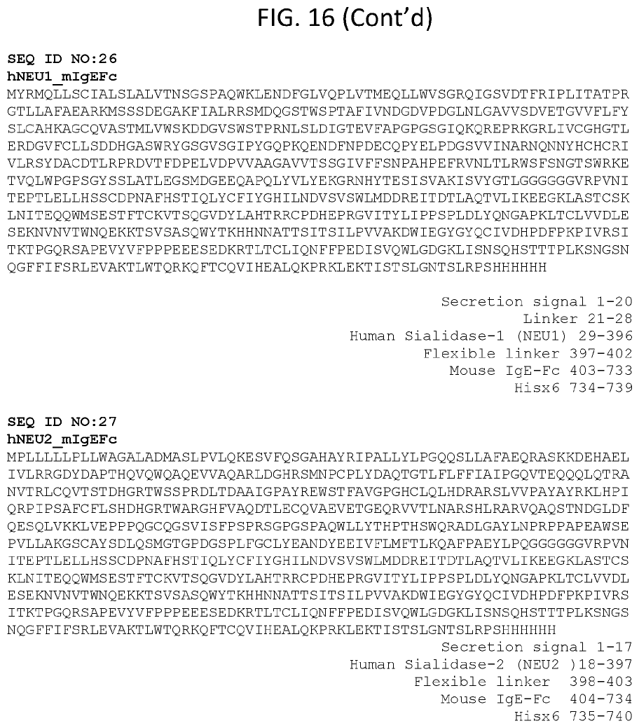

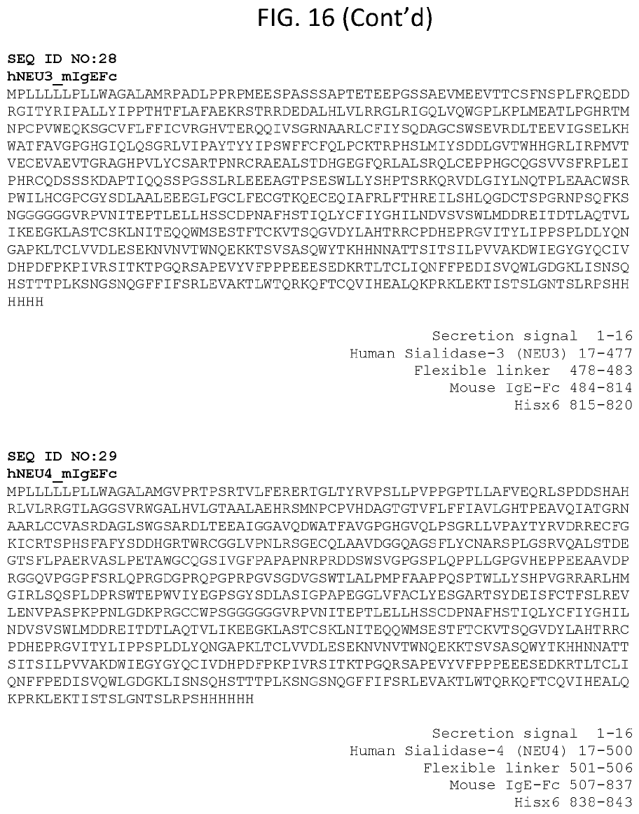

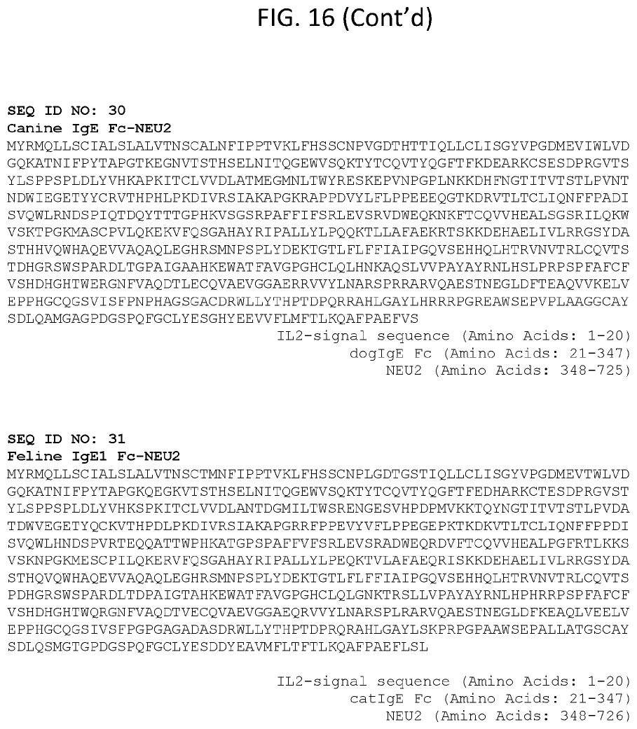

Described herein are fusion proteins comprising the Fc region of IgE, preferably human IgE, fused at the N or C terminus to a neuraminidase, referred to herein as Neu-IgE Fc Fusion Proteins. Exemplary sequences of Neu-IgE Fc Fusion Proteins are shown in FIG. 16. A schematic is shown in FIG. 4F.

The neuraminidases, e.g., full proteins or active portions thereof can be fused to IgE, or a part thereof. The neuraminidases can be fused to IgG Fc. Fc fusions have a number of advantageous: the soluble protein will have an extended serum half-life (e.g., more than 5 days, 10 days, 14 days, or 20 days), and also will form a dimer. In some embodiments, these fusion polypeptides can form homodimers or heterodimers, depending on the glycosylation target.

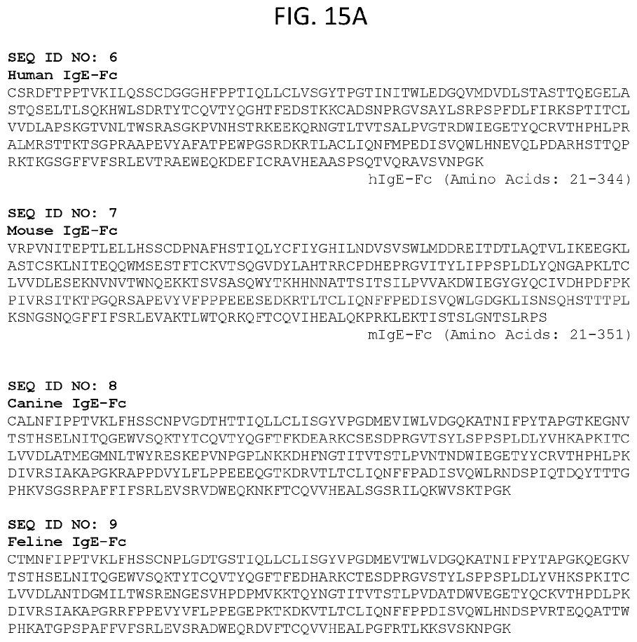

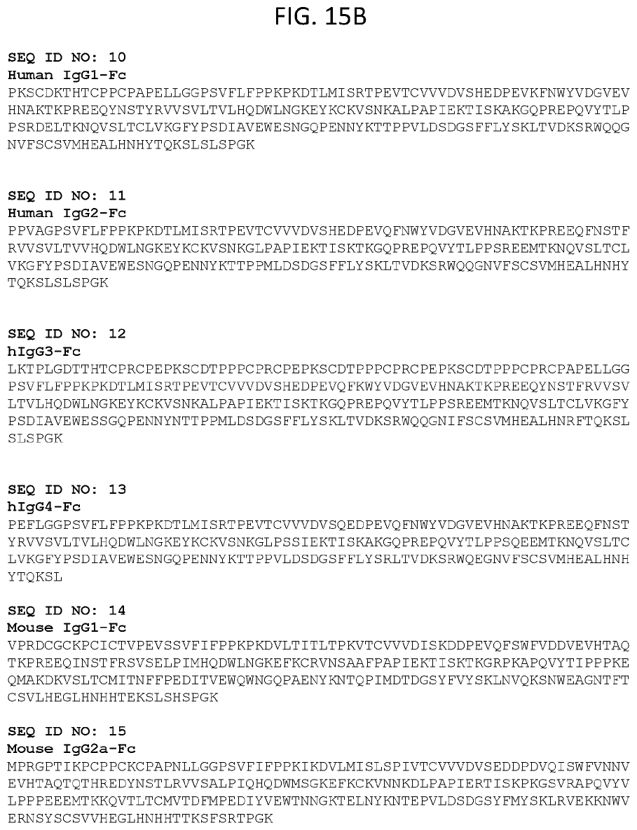

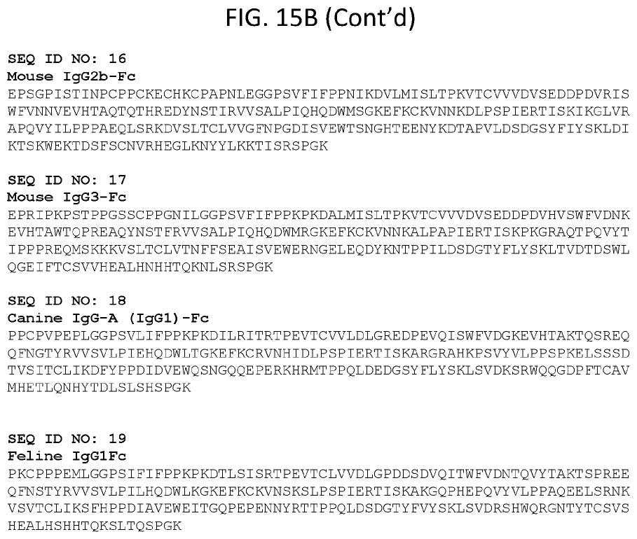

The IgE Fc can be the Fc region of any IgE known in the art. For example, the IgE Fc can be a human IgE-Fc (e.g., comprising SEQ ID NO: 6), a mouse IgE-Fc (e.g., comprising SEQ ID NO: 7), a canine IgE Fc (e.g., comprising SEQ ID NO: 8), or a feline IgE Fc (e.g., comprising SEQ ID NO: 9). See FIG. 15A. Preferably, the species of the immunoglobulins is chosen to correspond with the species of the subject to whom the fusion protein will be administered.

In some embodiments, the peptides comprise an IgE antibody epsilon chain CE2, CE3, and/or CE4 region, and an enzymatic luminal domains or a catalytic domain of neuraminidase (e.g., NEU1, NEU2, NEU3, NEU4, Vibrio cholerae serotype O1 sialidase). In some embodiments, the peptide has the amino acid sequence that is set forth in SEQ ID NOS: 1, 2, 3, 4, or 5, e.g., amino acids 48-415 of SEQ ID NO: 1, amino acids 1-380 (full length) of SEQ ID NO:2, amino acids 1-428 (full length) of SEQ ID NO:3, amino acids 1-484 (full length) of SEQ ID NO:4, amino acids 25-781 of SEQ ID NO: 5, or amino acids 557-747 of SEQ ID NO: 5.

Although fusion proteins comprising IgE Fc are exemplified herein, fusion proteins comprising IgG Fc are also described herein. Thus, in some embodiments the neuraminidase can be fused to IgG (e.g., IgG1, IgG2, IgG3, IgG4) or a part thereof. In some embodiments, the neuraminidase can be fused to the Fc portion of an IgG (e.g., IgG1, IgG2, IgG3, IgG4). Fc fusions have a number of advantageous: the soluble protein will have an extended serum half-life (e.g., more than 5 days, 10 days, 14 days, or 20 days), and also will form a dimer. In some embodiments, these fusion polypeptides can form homodimers or heterodimers, depending on the glycosylation target.

The IgG Fc can be the Fc region of any IgG known in the art. For example, the IgG Fc can be a human IgG1-Fc (e.g., comprising SEQ ID NO: 10), a human IgG2-Fc (e.g., comprising SEQ ID NO:11), a human IgG3-Fc (e.g., comprising SEQ ID NO: 12), a human IgG4-Fc (e.g., comprising SEQ ID NO: 13), a mouse IgG1-Fc (e.g., comprising SEQ ID NO: 14), a mouse IgG2a-Fc (e.g., comprising SEQ ID NO: 15), a mouse IgG2b-Fc (e.g., comprising SEQ ID NO: 16), a mouse IgG3-Fc (e.g., comprising SEQ ID NO: 17), a canine IgG-A Fc (e.g., comprising SEQ ID NO: 18), or a feline IgG1 Fc (e.g., comprising SEQ ID NO: 19). See, e.g., FIG. 15B. Preferably, the species of the immunoglobulins is chosen to correspond with the species of the subject to whom the fusion protein will be administered.

In some embodiments, these polypeptides can form a homodimer. The homodimer can have two enzymatic luminal domains (or catalytic domains) of mannosidase. In some other cases, the homodimer can have two enzymatic luminal domains (or catalytic domains) of sialidase or neuraminidase. In some embodiments, these polypeptides can form a heterodimer. In some embodiments, the heterodimer can have one enzymatic luminal domain (or catalytic domain) of mannosidase and one enzymatic luminal domain (or catalytic domain) of sialidase or neuraminidase.

In some embodiments, the peptides comprise an enzymatic luminal domain or a catalytic domain of sialidase or neuraminidase (e.g., NEU1, NEU2, NEU3, NEU4, Vibrio cholerae serotype O1 sialidase), and an IgE antibody heavy chain CH2 region, an IgE antibody heavy chain CH3 region, and/or an IgE antibody heavy chain CH3 region.

FIG. 16 shows several examples of glycosylation enzyme-Fc fusion proteins, including human NEU1-human IgE Fc (hNEU1-hIgE Fc, SEQ ID NO: 20); IgE Fc-NEU2 fusion protein (SEQ ID NO: 21); hNEU2-hIg EFc (SEQ ID NO:22); hNEU3-hIgE Fc (SEQ ID NO:23); hNEU4-hIgE Fc (SEQ ID NO:24) human Ig E Fc-Sialidase (Vibrio cholerae serotype O1 sialidase) fusion protein (SEQ ID NO: 25); human NEU1-mouse IgE Fc (hNEU1-mIgEFc, SEQ ID NO:26); human NEU2-mouse IgE Fc (hNEU2-mIgEFc, SEQ ID NO:27); hNEU3-mIgE Fc (SEQ ID NO:28); hNEU4-mIgE Fc (SEQ ID NO:29), canine IgE-NEU2 fusion protein (SEQ ID NO: 30), and feline IgE Fc-NEU2 fusion protein (SEQ ID NO: 31).

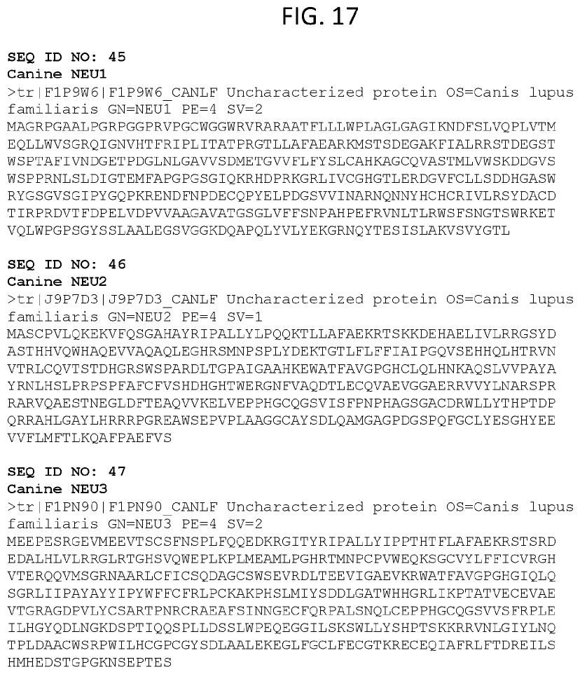

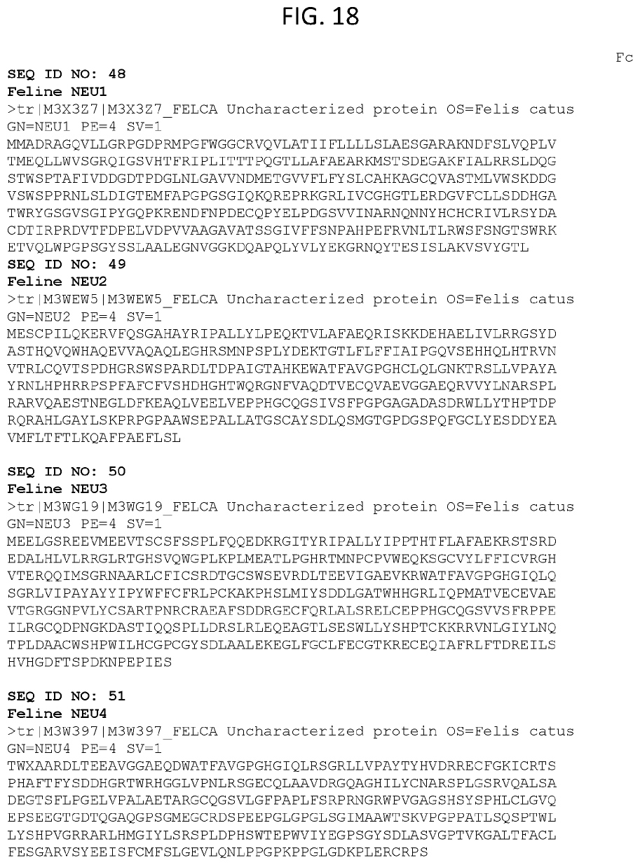

In some embodiments, the peptide can comprise IgE antibody heavy chain constant regions (e.g., CH1, CH2, CH3 and/or CH4) and/or glycosylation enzymes derived from non-human animals (e.g., dog, cat, cow, or horse; see FIGS. 17-20). Exemplary sequences In some embodiments, canine IgE-NEU2 has a sequence that is set forth in SEQ ID NO: 30, feline IgE1-NEU2 can have a sequence that is set forth in SEQ ID NO: 32.

In some embodiments, these peptides can additionally include signal sequences, e.g., IL2-signal sequence (e.g., MYRMQLLSCIALSLALVTNS, SEQ ID NO: 32), a secretion signal (e.g., MPLLLLLPLLWAGALA, SEQ ID NO:33), or κ-signal sequence (e.g., METDTLLLWVLLLWVPGSTGDAAQPARRAVRSLVPSSDP, SEQ ID NO: 34). These signal sequences usually present at the N-terminus of the peptides.

In some embodiments, the fusion proteins also include one or more flexible linkers. The linkers can be used to attach the separate parts of the fusion protein together. In some embodiments, the linker is a peptide linker. Peptide linkers can be from about 2-100, 10-50, or 15-30 amino acids long. In some embodiments, peptide linkers may be at least 2, 4, 5, 6, 10, 15, or at least 20 amino acids long and/or up to 20, 25, 35, 40, 60, 80, 90, or no more than 100 amino acids long. In some embodiments, the linker is a peptide linker comprising one or more glycines and/or serines, e.g., a single or repeating GGGGS (SEQ ID NO: 35), GGGS (SEQ ID NO: 36), GS, GGGGGG (SEQ ID NO: 37), GSGGS (SEQ ID NO: 38), GGSG (SEQ ID NO: 39), GGSGG (SEQ ID NO: 40), GSGSG (SEQ ID NO: 41), GSGGG (SEQ ID NO: 42), GGGSG (SEQ ID NO: 43), and/or GSSSG (SEQ ID NO: 44) sequence(s). Other linkers are known in the art. Intact antibodies with desired specificity can also be fused to glycosylation enzymes, enabling specific targeting of the enzymes. Further, similar protein fusions can be generated using dog/cat/horse/cow equivalent/homologous antibodies or glycosylation enzymes, enabling treatment of non-human animals (e.g., pets and livestock).

Glycoengineered Intravenous IgE (gIVIE)

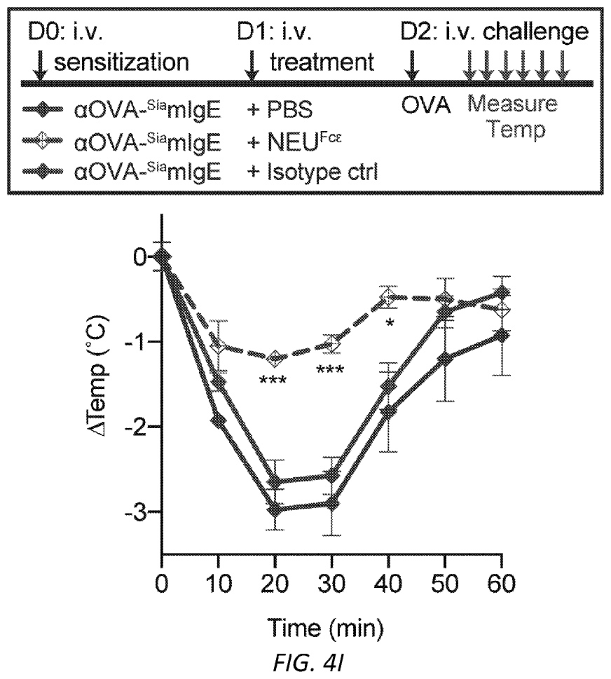

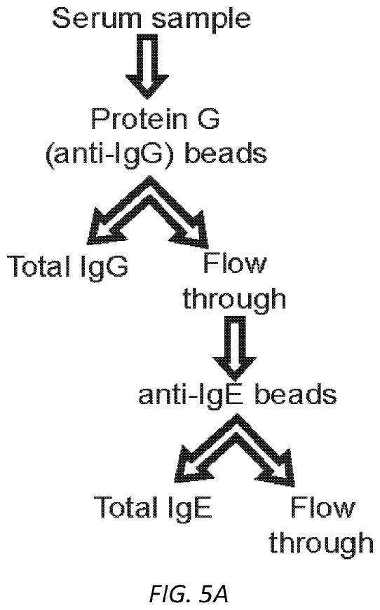

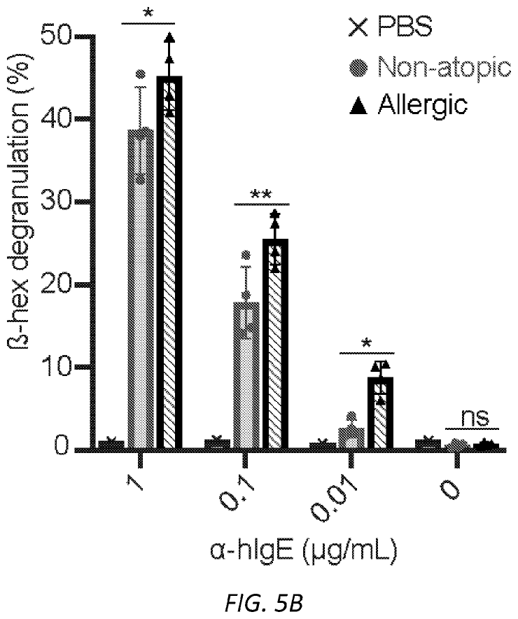

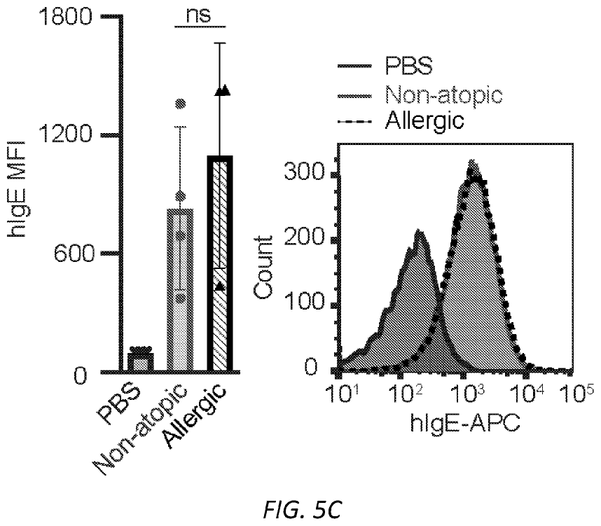

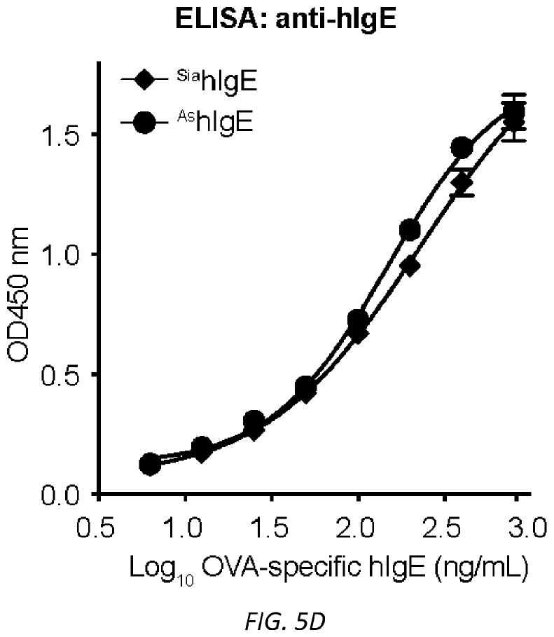

Also provided herein are glycoengineered intravenous IgE (gIVIE) compositions. Analogous to the intravenous immunoglobulin (IVIg) compositions presently in clinical use, the compositions can comprise normal polyspecific obtained from large numbers of healthy donors. The compositions can be polyclonal natural antibodies synthesized, in response to immune stimuli (antigens and T cells), by plasma B cells. Methods for the production of therapeutic IVIG compositions are known in the art (see, e.g., Afonso and Joao, Biomolecules. 2016 March; 6(1): 15 and references cited therein) and can be adapted for production of IVIE, e.g., as shown in FIG. 5A and described herein, or by other methods, e.g., as described in Kleine-Tebbe et al., J Immunol Methods. 1995 Feb. 27; 179(2):153-64. After obtaining the IgE, they are treated with sufficient neuraminidase (e.g., NEU1, NEU2, NEU3, NEU4, Vibrio cholerae serotype O1 sialidase) to remove sialic acid from the IgE, to produce a gIVIE composition, e.g., that attenuates IgE effector functions in vivo.

### IgE-Mediated Disorders

IgE is known to mediate allergic responses and is produced by B cells in both membrane-bound and secretory form. IgE binds to B-cells through its Fc region to a low affinity IgE receptor, known as FcεRII. Upon exposure to an allergen, B-cells bearing a surface-bound IgE molecule specific for the allergen are activated and further develop into IgE-secreting plasma cells. The secreted IgE molecules, which are specific for the allergen, circulate through the bloodstream and become bound to the surface of mast cells in tissue and basophils in bloodstream through the high affinity receptor, known as FcεRI. This binding by allergen-specific IgE, sensitizes the mast cells and basophils for the allergen. Subsequent exposure to the allergen causes cross-linking of FcεRI on basophils and mast cells, leading to up-regulation of the granular molecule CD63 and the release of a number of factors, such as histamine, platelet activating factors, eosinophil and neutrophil chemotactic factors, and cytokines such as IL-3, IL-4, IL-5 and GM-CSF.

As used herein, the term “IgE-mediated response” refers to responses of IgE receptor expressing cells (e.g., basophils and mast cells) induced directly or indirectly by IgE. In some embodiments, the response can be observed (e.g., degranulation) and/or measured by up-regulation of the granular molecule CD63, or the release of one or more of histamine, platelet activating factors, eosinophil and neutrophil chemotactic factors, and cytokines such as IL-3, IL-4, IL-5 and GM-CSF. In some embodiments, IgE-mediated responses include e.g., degranulation, up-regulation of the granular molecule CD63, and/or the release of histamine from basophils. In some embodiments, IgE-mediated responses can cause allergic reactions.

As used herein, the term “attenuating an IgE-mediated response” refers to the extent, occurrence and/or frequency of an IgE-mediated response that is reduced by the methods as described herein, e.g., by administering an agent as described herein as compared to without administering the agent. The extent of reduction can be statistically significant and in certain embodiments, by at least 15%, 20%, 25%, 30%, 35%, 40%, 50%, 60%, 70%, 80%, 90% or greater.

The IgE-mediated disorder is characterized by abnormal responses mediated by IgE. In some embodiments, the abnormal responses mediated by IgE are due to overproduction of IgE and/or hypersensitivity of basophils or mast cells to IgE. Thus, IgE-mediated disorders include, e.g., (1) allergic disorders (e.g., asthma, atopic dermatitis, allergic rhinitis, allergic conjunctivitis, eczema, urticaria, food allergy and seasonal allergy, as well as anaphylactic shock); (2) autoimmune disorders (e.g., lupus, rheumatoid arthritis, psoriasis); and (3) anaphylaxis, etc. A detailed description regarding IgE-mediated disorder and IgE-mediated response can be found, e.g., in U.S. Pat. No. 8,828,394 B2, which is incorporated herein by reference in its entirety.

IgE that can specifically recognize an allergen has a unique long-lived interaction with its high-affinity receptor FcεRI so that basophils and mast cells, capable of mediating inflammatory reactions, become “primed”, ready to release chemicals like histamine, leukotrienes, and certain interleukins. These chemicals cause many of the symptoms associated with allergy, such as airway constriction in asthma, local inflammation in eczema, increased mucus secretion in allergic rhinitis, and increased vascular permeability, which allow other immune cells to gain access to tissues, but which can lead to a potentially fatal drop in blood pressure as in anaphylaxis.

Anaphylaxis is a serious allergic reaction that is rapid in onset and may cause death. It typically causes e.g., an itchy rash, throat or tongue swelling, shortness of breath, vomiting, lightheadedness, and low blood pressure. These symptoms typically come on over minutes to hours. When anaphylaxis occurs, IgE binds to the antigen. The antigen-bound IgE then activates FcεRI receptors on mast cells and basophils. This leads to the release of inflammatory mediators such as histamine. These mediators subsequently increase the contraction of bronchial smooth muscles, trigger vasodilation, increase the leakage of fluid from blood vessels, and cause heart muscle depression.

As histamine is central to the pathogenesis of allergic disorders, e.g., asthma and atopic dermatitis, by attenuating IgE-mediated responses such as histamine release, the present method is also effective in treating allergic disorders.

Thus in some embodiments, the fusion proteins described herein can be used to target FcεRI-expressing cells.

### Methods of Treatment

The methods described herein include methods for treating IgE-mediated disorders, e.g., allergies, e.g., anaphylactic allergies, and methods for attenuating IgE-mediated responses. Generally, the methods include administering a therapeutically effective amount of compositions comprising Neu-IgE Fc fusion proteins or gIVIE as described herein, to a subject who is in need of, or who has been determined to be in need of, such treatment. In some embodiments, the subject can be allergic to a food antigen, e.g., eggs, milk, peanuts, soy, fish, shellfish, tree nuts, and/or wheat, or to an environmental allergen, e.g., dust mite excretions, pollen, pet dander, or royal jelly, inter alia. See, e.g., Valenta et al., Gastroenterology. 2015 May; 148(6): 1120-1131.e4.

As used in this context, to “treat” means to ameliorate at least one symptom of the disorders or the diseases. Often, the treatment results in an improvement in the symptoms. In some embodiments, the treatment can result in a reduction of histamine release. In some embodiments, one or more of the clinical symptoms are ameliorated or reduced, the duration being shortened, the frequency of the occurrence of the symptoms is reduced, or the clinical symptoms are prevented from manifesting.

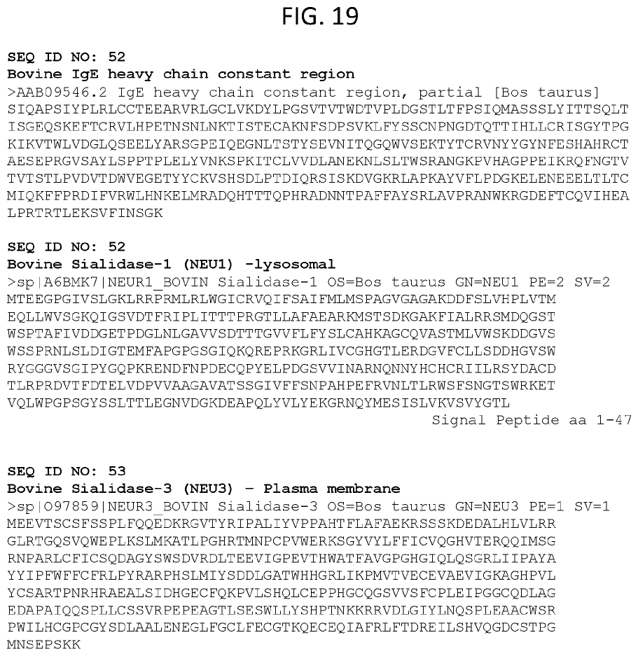

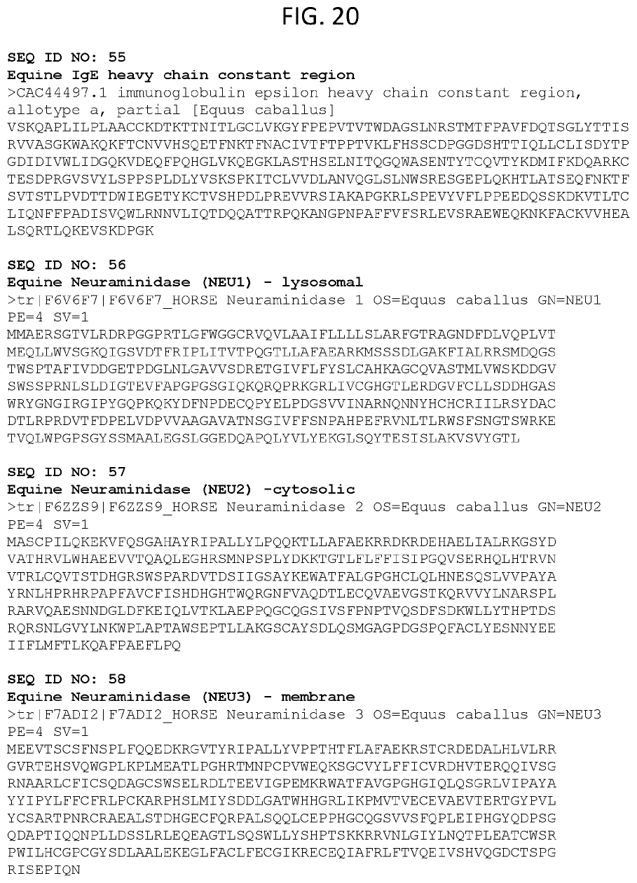

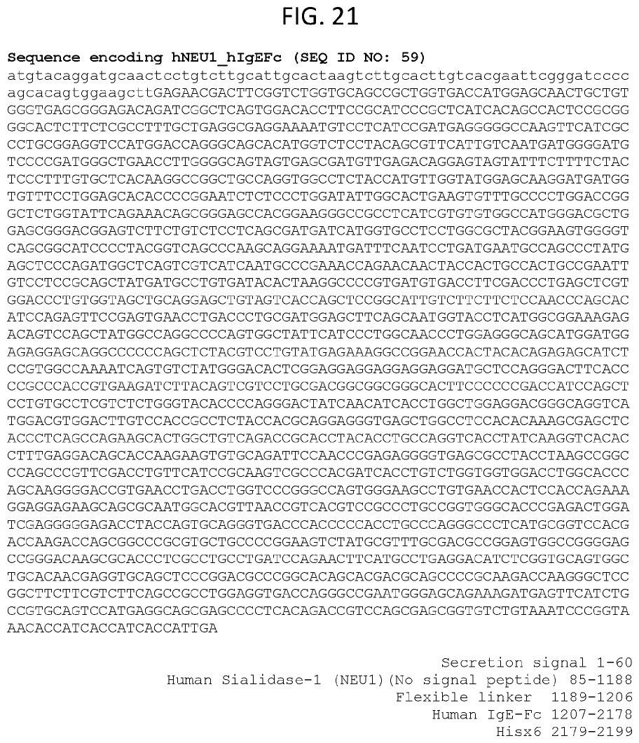

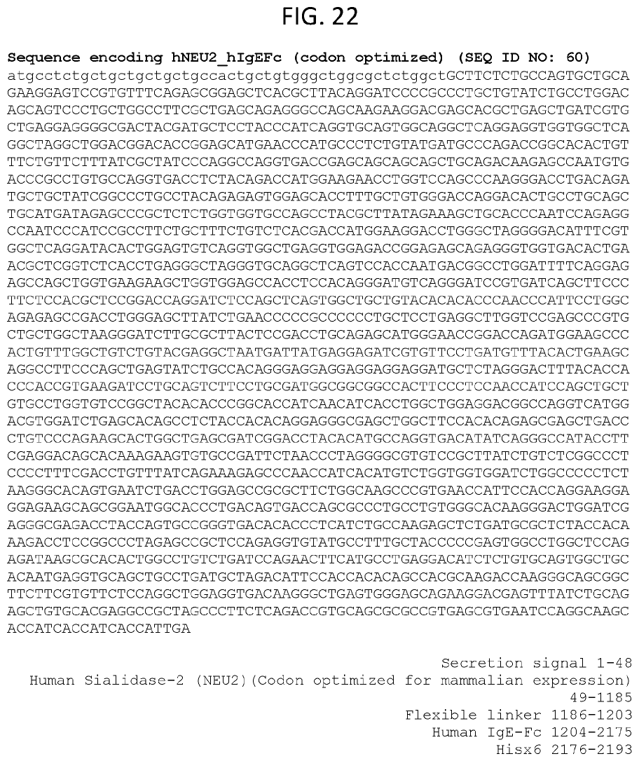

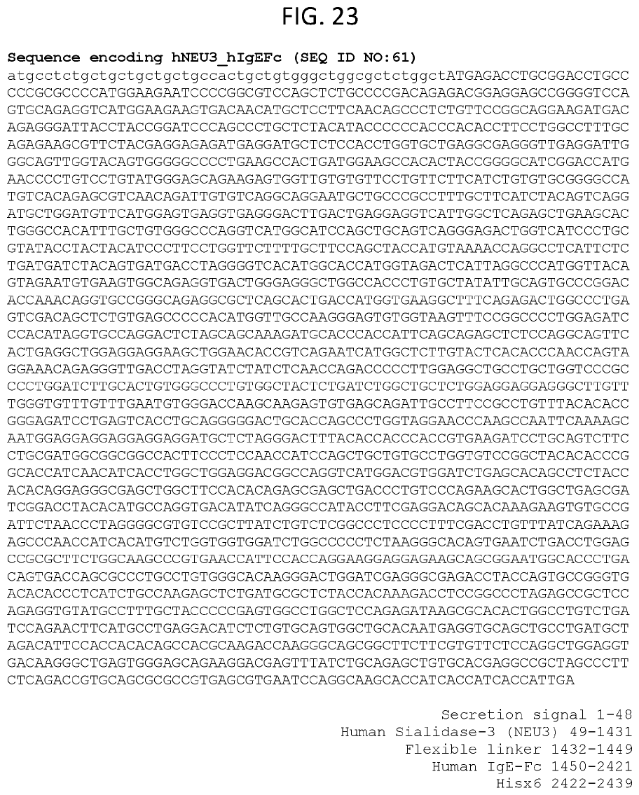

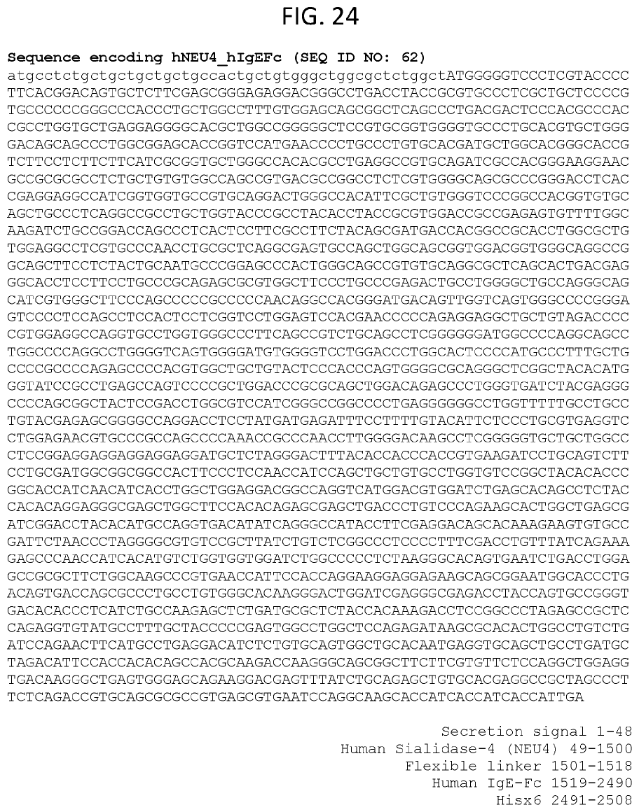

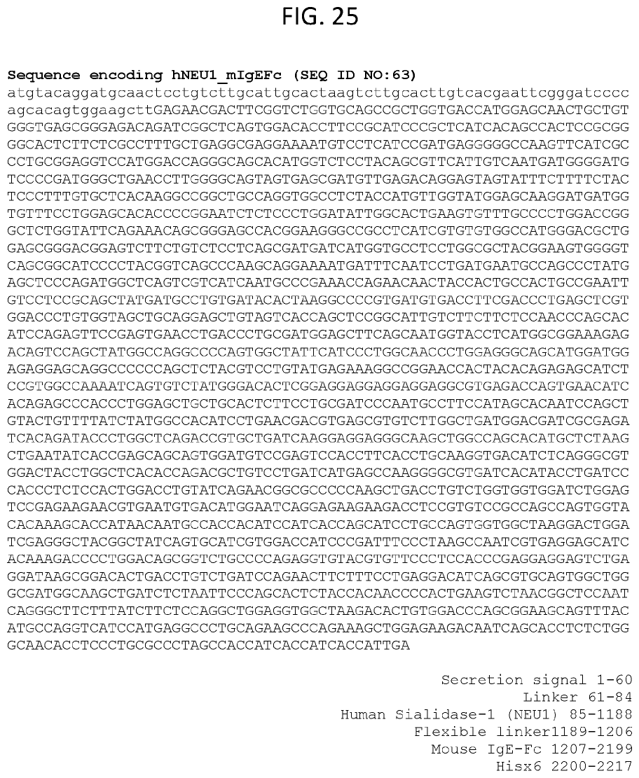

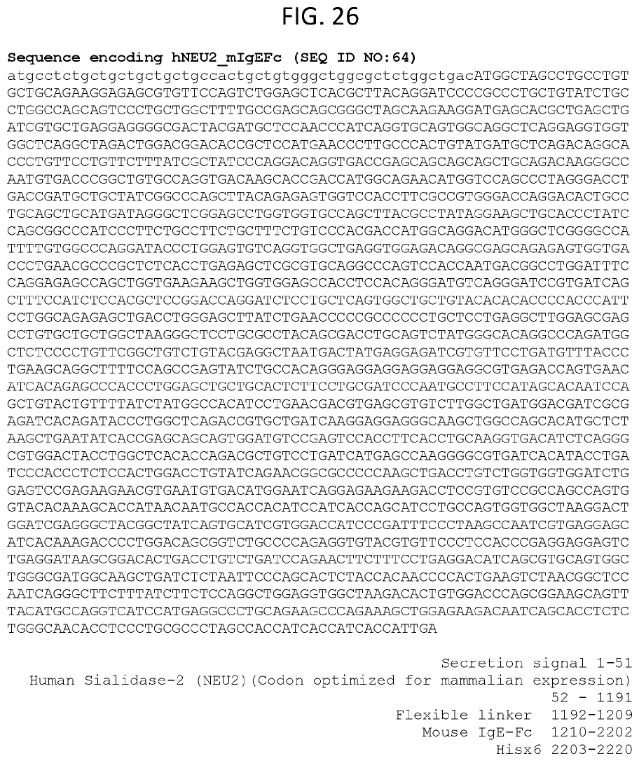

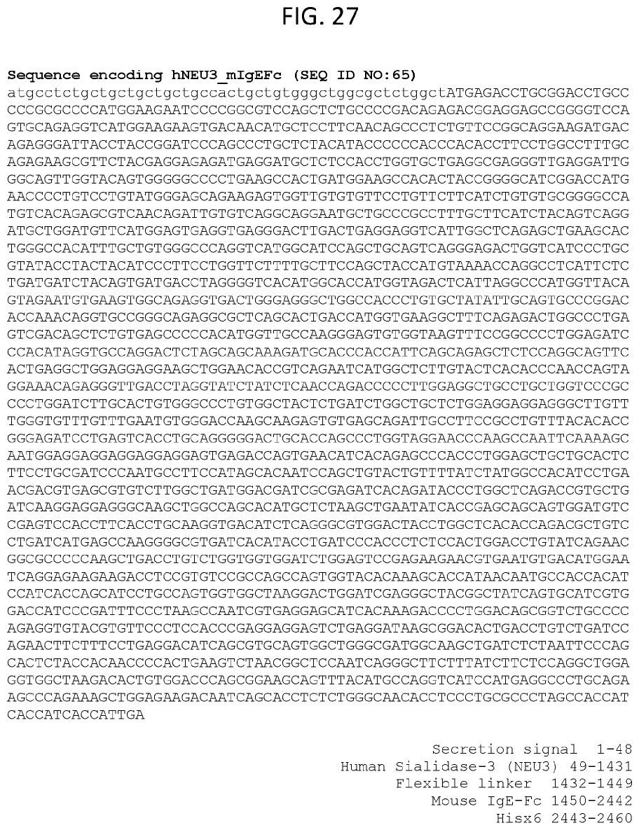

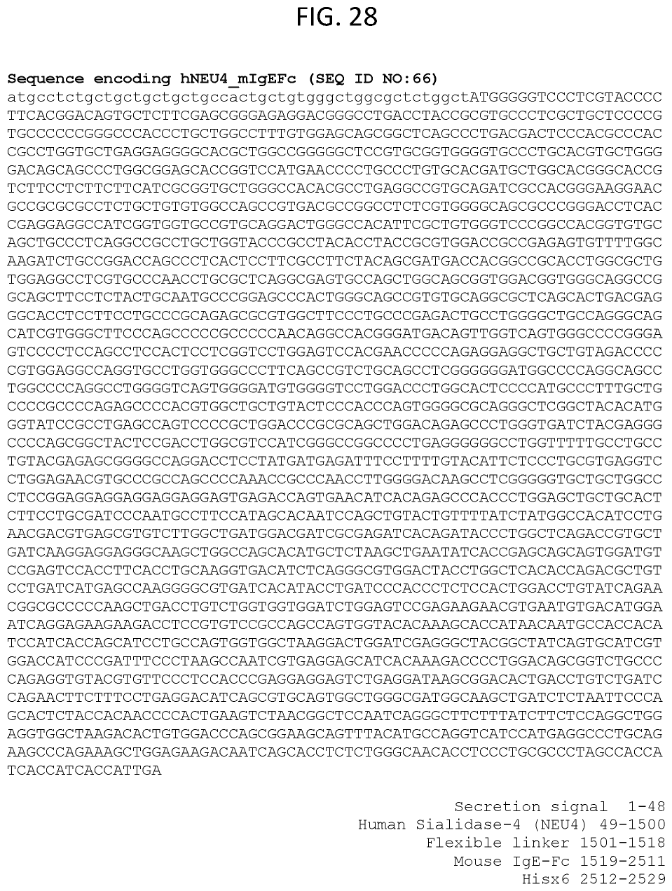

As used herein, the terms “subject” and “patient” are used interchangeably throughout the specification and describe an animal, human or non-human, e.g., a mammal, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals. Thus, in some embodiments, the glycosylation enzymes, the antibodies, or the parts thereof (e.g., Fc regions of the antibodies or the catalytic domain of the glycosylation enzymes) as described herein can also derive from these non-human animals. The present disclosure further provides the amino acid sequences of the glycosylation enzymes, and the antibodies or the parts thereof that derive from some of these non-human animals. For example, FIGS. 15A-B and 17 list exemplary amino acid sequences of dog IgE and IgG heavy chain constant regions, dog NEU1, dog NEU2, and dog NEU3. FIGS. 15A-B and 18 list exemplary amino acid sequences of cat IgE and IgG heavy chain constant regions, cat NEU1, cat NEU2, cat NEU3 and cat NEU4. FIG. 19 lists exemplary amino acid sequences of cow IgE heavy chain constant region, cow NEU1, and cow NEU3. FIG. 20 lists exemplary amino acid sequences of horse IgE heavy chain constant region, horse NEU1, horse NEU2, and horse NEU3.

In some embodiments, the subject is a human (e.g., male human or female human) with an age over 6 months old, 12 months old, 2 years old, 5 years old, 6 years old, 10 years old, 12 years old, 16 years old, 18 years old, 25 years old, 30 years old, 40 years old, 50 years old, 60 years old, 70 years old, or 80 years old.

As used herein, the terms “therapeutically effective” and “effective amount”, used interchangeably, applied to a dose or amount refers to a quantity of a composition, compound or pharmaceutical formulation that is sufficient to result in a desired activity upon administration to a subject in need thereof. Within the context of the present disclosure, the term “therapeutically effective” refers to that the composition, compound or pharmaceutical formulation, in a sufficient amount, can reduce or eliminate at least one symptom or one condition of the disorders as described herein, delay or reduce risk or frequency of symptoms, or delay or reduce risk of progression.

### Expression Systems

To use the fusion proteins or peptides as described herein, it may be desirable to express them from a nucleic acid that encodes them. This can be performed in a variety of ways. For example, the nucleic acid encoding the fusion proteins or peptides can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the fusion proteins or peptides for production. The nucleic acid encoding the fusion proteins or peptides can also be cloned into an expression vector, for administration to a plant cell, animal cell, preferably a mammalian cell or a human cell, fungal cell, bacterial cell, or protozoan cell.

To obtain expression, a sequence encoding a fusion protein or peptide is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available. In some embodiments, the fusion proteins and peptides are expressed by transfection of HEK-293T cells, Expi293 cells, or CHO cells with vectors comprising the polynucleotides encoding fusion proteins and peptides as described in this disclosure.

The promoter used to direct expression of a nucleic acid depends on the particular application. For example, a strong constitutive promoter is typically used for expression and purification of fusion proteins. In contrast, when a vector encoding the fusion protein or peptide is to be administered in vivo, either a constitutive or an inducible promoter can be used, depending on the particular need. In some embodiments, the promoter for administration of the vector encoding the fusion protein or peptide can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the fusion protein or peptide, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the fusion protein or peptide can include RNA Pol III promoters to drive expression of the guide RNAs, e.g., the H1, U6 or 7SK promoters. These human promoters allow for expression of fusion protein or peptide in mammalian cells following plasmid transfection.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the fusion protein or peptide.

The present disclosure also includes the vectors and cells comprising the vectors, as well as kits comprising the proteins and nucleic acids described herein, e.g., for use in various methods as described herein.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms.

An effective amount can be administered in one or more administrations, applications or dosages. A therapeutically effective amount of polypeptides, multimers, or compositions (i.e., an effective dosage) depends on the polypeptides, multimers, or compositions that are selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the polypeptides, multimers, or compositions described herein can include a single treatment or a series of treatments.

Dosage, toxicity and therapeutic efficacy of the polypeptides, multimers, or compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Polypeptides, multimers, or compositions which exhibit high therapeutic indices are preferred. While polypeptides, multimers, or compositions that exhibit toxic side effects may be used, care should be taken to design a delivery system that targets polypeptides, multimers, or compositions to the site of affected tissue in order to minimize potential damage to uninfected cells and, thereby, reduce side effects.

The data obtained from cell culture assays and animal studies can be used in formulating a range of dosage for use in humans. The dosage of polypeptides, multimers, or compositions lies preferably within a range of circulating concentrations that include the ED50 with little or no toxicity. The dosage may vary within this range depending upon the dosage form employed and the route of administration utilized. For any polypeptides, multimers, or compositions used in the methods as described in this disclosure, the therapeutically effective dose can be estimated initially from cell culture assays. A dose may be formulated in animal models to achieve a circulating plasma concentration range that includes the IC50 (i.e., the concentration of the test polypeptide, multimer, or composition which achieves a half-maximal inhibition of symptoms) as determined in cell culture. Such information can be used to more accurately determine useful doses in humans. Levels in plasma may be measured, for example, by high performance liquid chromatography.

### Pharmaceutical Compositions and Methods of Administration

The methods described herein include the use of pharmaceutical compositions comprising fusion proteins as described in this disclosure as an active ingredient as well as the compositions themselves.

Pharmaceutical compositions typically include a pharmaceutically acceptable carrier. As used herein, the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating polypeptides, multimers, or compositions as described in this disclosure in the required amount in an appropriate solvent with one or a combination of ingredients enumerated above, as required, followed by filtered sterilization. Generally, dispersions are prepared by incorporating the polypeptides, multimers, or compositions into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active agents can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or agents of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the composition can be delivered in the form of an aerosol spray from a pressured container or dispenser that contains a suitable propellant, e.g., a gas such as carbon dioxide, or a nebulizer. Such methods include those described in U.S. Pat. No. 6,468,798.

In some embodiments, the polypeptides or multimers are prepared with carriers that will protect the polypeptides or multimers against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### Methods of Diagnosis

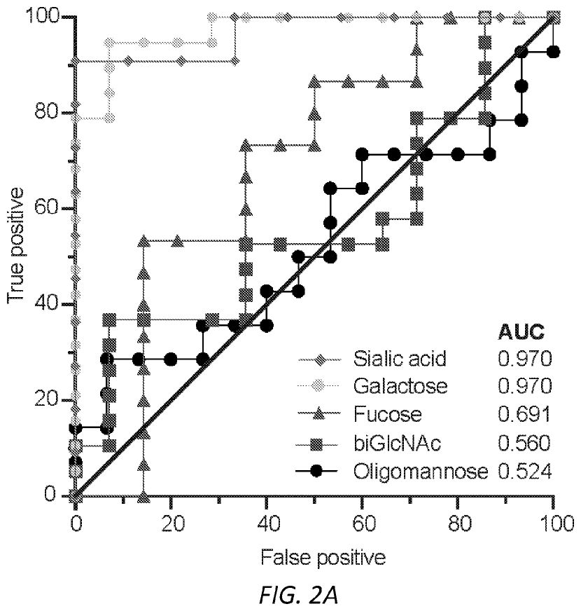

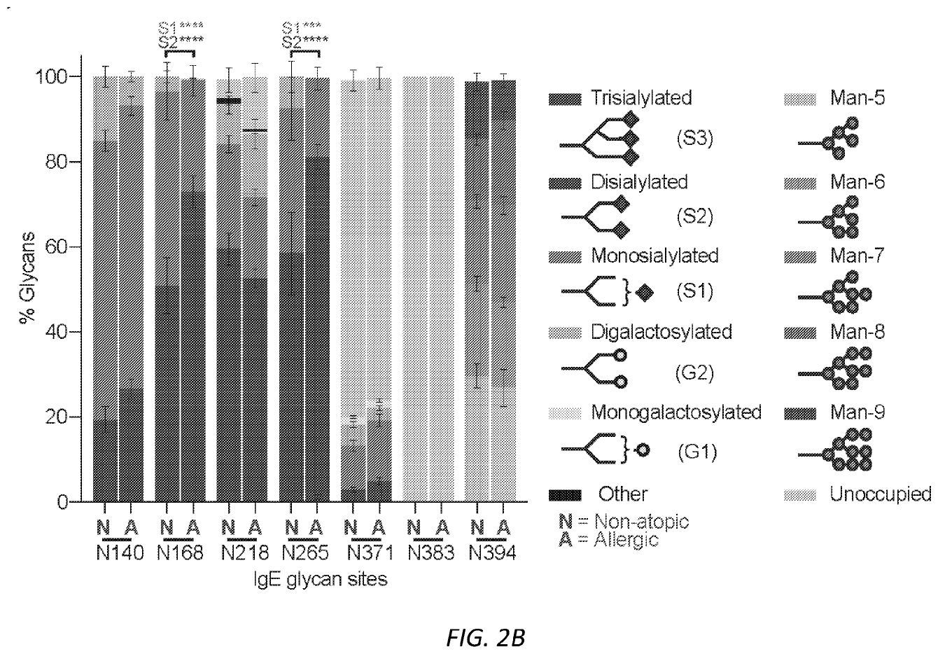

Included herein are methods for diagnosing allergy. The methods rely on detection of a sialylation levels on IgE. The methods include obtaining a sample from a subject, and evaluating the presence and/or level of sialylation on IgE, e.g., on total IgE, or on allegen-specific IgE (i.e., IgE that binds specifically to a selected allergen) in the sample, and comparing the presence and/or level with one or more references, e.g., a control reference that represents a normal level of sialylation on IgE e.g., a level in an unaffected (non-allergic) subject, and/or a disease reference that represents a level of the proteins associated with allergy e.g., a level in a subject having an allergy, e.g., an anaphylactic allergy. Suitable reference values can include those shown in FIGS. 1I, and 2B, showing total IgE titers and Ara h 2 titers.

As used herein the term “sample”, when referring to the material to be tested for the presence of a biological marker using the method of the invention, includes whole blood, plasma, or serum. The type of sample used may vary depending upon the clinical situation in which the method is used. Various methods are well known within the art for the identification and/or isolation and/or purification of IgE from a sample. In some embodiments, the methods include isolating antigen-specific IgE, e.g., by purifying total IgE, and then enriching antigen/allergen-specific IgE using antigen/allergen-coupled beads.

The presence and/or level of sialylation on IgE can be evaluated using methods known in the art, e.g., using standard electrophoretic and quantitative immunoassay methods, including but not limited to, Western blot; enzyme linked immunosorbent assay (ELISA); radio-immunoassay; immunohistochemistry (IHC); or mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687).

In some embodiments, an ELISA method may be used, wherein the wells of a mictrotiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

Mass spectrometry, and particularly matrix-assisted laser desorption/ionization mass spectrometry (MALDI-MS) and surface-enhanced laser desorption/ionization mass spectrometry (SELDI-MS), is useful for the detection of biomarkers of this invention. (See U.S. Pat. Nos. 5,118,937; 5,045,694; 5,719,060; 6,225,047)

In some embodiments, the presence and/or level of sialylation on IgE is comparable to the presence and/or level of the protein(s) in the disease reference, and the subject has or has had one or more symptoms associated with an allergic reaction, then the subject can be diagnosed with an allergy, e.g., an anaphylactic allergy. In some embodiments, the subject has no overt signs or symptoms of allergy or allergic reaction, but the presence and/or level of sialylation on IgE is comparable to the presence and/or level of the protein(s) in the disease reference, then the subject has an increased risk of developing an allergy, e.g., an anaphylactic allergy. In some embodiments, once it has been determined that a person has an allergy, e.g., an anaphylactic allergy, or has an increased risk of developing an allergy, e.g., an anaphylactic allergy, then a treatment, e.g., as known in the art or as described herein, can be administered.

Suitable reference values can be determined using methods known in the art, e.g., using standard clinical trial methodology and statistical analysis. The reference values can have any relevant form. In some cases, the reference comprises a predetermined value for a meaningful level of sialylation on IgE, e.g., a control reference level that represents a normal level of sialylation on IgE, e.g., a level in an unaffected subject or a subject who is not at risk of developing an allergy as described herein, and/or a disease reference that represents a level of the proteins associated with conditions associated with allergy or anaphylactic allergy, e.g., a level in a subject having an allergy (e.g., an anaphylactic allergy).

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have a disorder described herein (e.g. an allergy, e.g., an anaphylactic allergy). In some cases it may be desirable that the control subject is non-allergic, and in other cases it may be desirable that a control subject has an allergy, e.g., to a different allergen, or a non-anaphylactic allergy.

A disease reference subject is one who has (or has an increased risk of developing) an allergy, e.g., an anaphylactic allergy. An increased risk is defined as a risk above the risk of subjects in the general population.

Thus, in some cases the level of sialylation on IgE in a subject being less than or equal to a reference level of sialylation on IgE is indicative of a clinical status (e.g., indicative of a disorder as described herein, e.g., an allergy, e.g., an anaphylactic allergy. In other cases the level of sialylation on IgE in a subject being greater than or equal to the reference level of sialylation on IgE is indicative of the absence of disease or normal risk of the disease. In some embodiments, the amount by which the level in the subject is the less than the reference level is sufficient to distinguish a subject from a control subject, and optionally is a statistically significantly less than the level in a control subject. In cases where the level of sialylation on IgE in a subject being equal to the reference level of sialylation on IgE the “being equal” refers to being approximately equal (e.g., not statistically different).

The predetermined value can depend upon the particular population of subjects (e.g., human subjects) selected. For example, an apparently healthy non-allergic population may have a different ‘normal’ range of levels of sialylation on IgE than will a population of subjects which have, are likely to have, or are at greater risk to have, an allergy, e.g., an anaphylactic allergy. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

Upon diagnosis with an allergy, e.g., an anaphylactic allergy, the subject can be administered or prescribed a treatment, e.g., avoidance of the allergen, immunotherapy (e.g., oral, sublingual, or subcutaneous immunotherapy, e.g., Sublingual immunotherapy (SLIT)), and/or a pharmacological treatment, e.g., a chronically administered treatment (e.g., corticosteroids, antihistamines, Leukotriene receptor antagonists (LTRAs), Anti-IgE antibody) or an acutely-administered treatment (e.g., epinephrine or a rapid-acting bronchodilator). See, e.g., Min, Allergy Asthma Immunol Res. 2010 April; 2(2): 65-76.

In some embodiments, the methods rely on the observation that Human lgE has seven N-linked glycosylation sites, 5 of which are occupied by complex biantennary glycans. One site is occupied by an oligomannose glycan, and one site is unoccupied. On complex biantennary glycans, sialic acid is attached to galactose. Thus provided herein is an in vitro assay method to determine the pathogenicity of circulating lgEs in allergic humans. The method can include measuring the levels of terminal sialic acid sugar residues or terminal galactose residues on sera lgEs, isolated from said humans, wherein higher levels of sialylation (higher sialylation correlates with less terminal galactose, and vice versa) predict susceptibility to a pathogenic reaction (e.g., anaphylaxis) in said allergic humans. In some embodiments, the in vitro assay is an ELISA in which total lgE is captured, and sialylation levels are quantified by the ratio of the amount of lgE-bound labelled lectin, specific for terminal sialic acid or terminal galactose, normalized to the amount of total anti-lgE detection antibody is bound. In some embodiments, the measurement for the amount of lgE-bound labelled lectin is by, but not limited to, fluorescence or a colorimetric enzymatic reaction. Also provided is an in vitro assay method to determine the pathogenicity to a specific allergen of circulating lgEs in humans; the method can include measuring the levels of sialic acid sugar residues or galactose residues on said lgEs, isolated from the sera of human patients and which bind to a specific allergen, wherein higher levels of sialylation on allergen-bound lgEs predict susceptibility to a pathogenic reaction (e.g. anaphylaxis) to said allergen. In some embodiments, the in vitro assay is an ELISA in which allergen-specific lgE is captured, and sialylation levels are quantified by the ratio of the amount of lgE-bound labelled lectin, specific for terminal sialic acid or terminal galactose, normalized to the amount of total anti-lgE detection antibody is bound. In some embodiments, the measurement for the amount of lgE-bound labelled lectin is by, but not limited to, fluorescence or a colorimetric enzymatic reaction.

### EXAMPLES

The invention is further described in the following examples, which do not limit the scope of the invention described in the claims.

### Example 1. IgE Sialylation is a Determinant of Allergic Pathogenicity

### Methods

The following materials and methods were used in this Example.

### IgE Antibodies

All human samples were collected under IRB approved protocols. Serum samples from MGH IRB approved and consented peanut allergic were collected prior to treatment. Peanut allergy was confirmed by clinical history, allergen-specific IgE screening, and double-blind placebo-controlled oral challenge (PNOIT2, NCT01750879, Table 1). IRB-approved non-atopic adults were recruited (Research Blood Components, Boston, Mass.) on the basis of self-identification as non-allergic donors. Non-atopy was confirmed by clinical history, and allergen-specific IgE screening (Table 1). Total IgE, Ara h 2-specific IgE, Fel d 1-specific IgE, Der p 1-specific IgE, and Bet v 1-specific IgE were determined by ImmunoCap Assay (Phalleon, Thermo Scientific) according to manufacturer's protocols. Primary IgE was enriched from serum samples by serially depleting IgG by protein G agarose (GE Healthcare) followed by anti-IgE conjugated NHS-beads (GE Healthcare). IgE purity was confirmed by protein electrophoresis and coomassie gel staining. Recombinant OVA-specific IgE was generated as described²³. Briefly, cDNA sequences for generating OVA-specific heavy ε and light κ chain of mouse and human IgE²³ were cloned into pcDNA3.4 using restriction enzyme sites Xbal and AgeI. To generate recombinant OVA-specific mouse or human IgE, plasmids containing OVA-specific heavy and light chain were transiently co-transfected at 1:1 ratio using Expi293 Expression System Kit (Life Technologies) according to the manufacturer's protocol. The cells expressing IgE were selected by addition of 400 μg/mL G418 in the culture media for two weeks and maintained before expanding to a larger scale production. OVA-specific IgE was purified from cell culture supernatant by OVA-coupled agarose beads²³.

TABLE 1
Patient Demographic Data

| Sample # | Diagnosis | Gender | Age | Other Atopies* |
|---|---|---|---|---|
| 73 | Allergic | M | 22 | Yes |
| 10 | Allergic | F | 25 | Yes |
| 22 | Allergic | F | 19 | Yes |
| 34 | Allergic | F | 30 | Yes |
| 51 | Allergic | F | 15 | Yes |
| 60 | Allergic | F | 40 | Yes |
| 61 | Allergic | F | 27 | Yes |
| 67 | Allergic | F | 52 | Yes |
| 84 | Allergic | M | 22 | Yes |
| 97 | Allergic | F | 36 | Yes |
| 24 | Allergic | M | 36 | Yes |
| 33 | Allergic | F | 30 | Yes |
| 34 | Allergic | F | 16 | Yes |
| 69 | Allergic | M | 15 | Yes |
| 80 | Allergic | F | 8 | Yes |
| 95 | Allergic | M | 8 | Yes |
| 97 | Allergic | F | 36 | Yes |
| 100 | Allergic | F | 22 | Yes |
| 105 | Allergic | F | 22 | Yes |
| 111 | Allergic | F | 22 | Yes |
| 106 | Allergic | F | 32 | Yes |
| 149 | Non-atopic | M | 27 | No |
| 349 | Non-atopic | F | 35 | No |
| 241 | Non-atopic | M | 29 | No |
| 528 | Non-atopic | M | 38 | No |
| 53208 | Non-atopic | M | 36 | No |
| 53209 | Non-atopic | M | 37 | No |
| 53210 | Non-atopic | M | 39 | No |
| 53211 | Non-atopic | F | 31 | No |
| 53195 | Non-atopic | M | 60 | No |
| 57543 | Non-atopic | M | 69 | No |
| 57544 | Non-atopic | F | 27 | No |
| 57546 | Non-atopic | M | 22 | No |
| 57699 | Non-atopic | M | 58 | No |
| 57713 | Non-atopic | M | 32 | No |
| 57714 | Non-atopic | M | 28 | |
| 56986 | Non-atopic | M | 29 | |
| 56988 | Non-atopic | F | 50 | |
| 57527 | Non-atopic | F | 29 | |

*Includes reports of allergic asthma, allergic rhinitis, other food dermatitis.

### ELISAs

Sandwich ELISA for quantifying mIgE and OVA-specific binding were conducted as previously described²³. Briefly, 96-well Nunc plates were coated with goat polyclonal anti-mouse IgE (Bethyl Laboratories) or OVA and blocked with BSA in PBS (1% BSA for mIgE and 2% for OVA) prior to sample incubation. Samples were probed with goat polyclonal anti-mouse IgE-HRP (2 ng/ml; Bethyl Laboratories) and the reactions were detected by 3,3,5,5-tetramethylbenzidine (TMB; Thermo Fisher Scientific) and stopped by 2 M sulfuric acid, and the absorbance was measured at 450 nm.

### Glycopeptide Mass Spectrometry and Glycan Analysis

Site specific glycosylation was quantified for IgE isolated from non-allergic donors and from peanut allergic donors using nano LC-MS/MS following enzymatic digestion of the proteins as described previously, with minor modifications²²⁻²⁴ (Tables 2, 3).

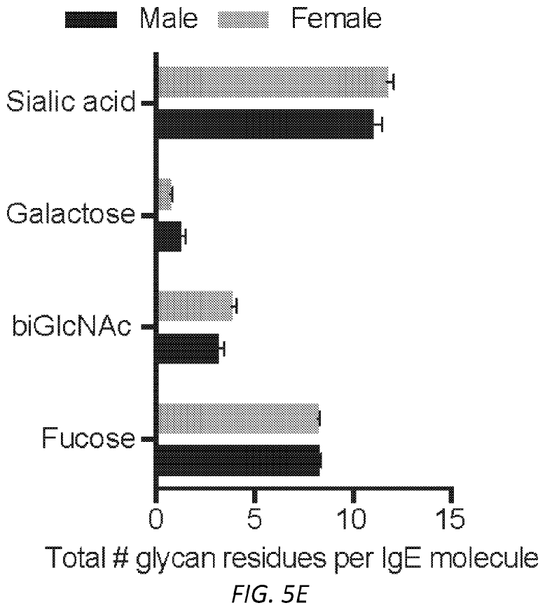

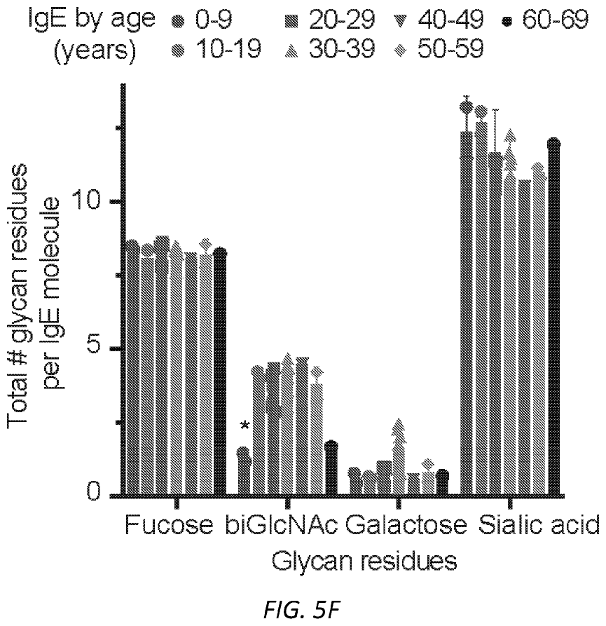

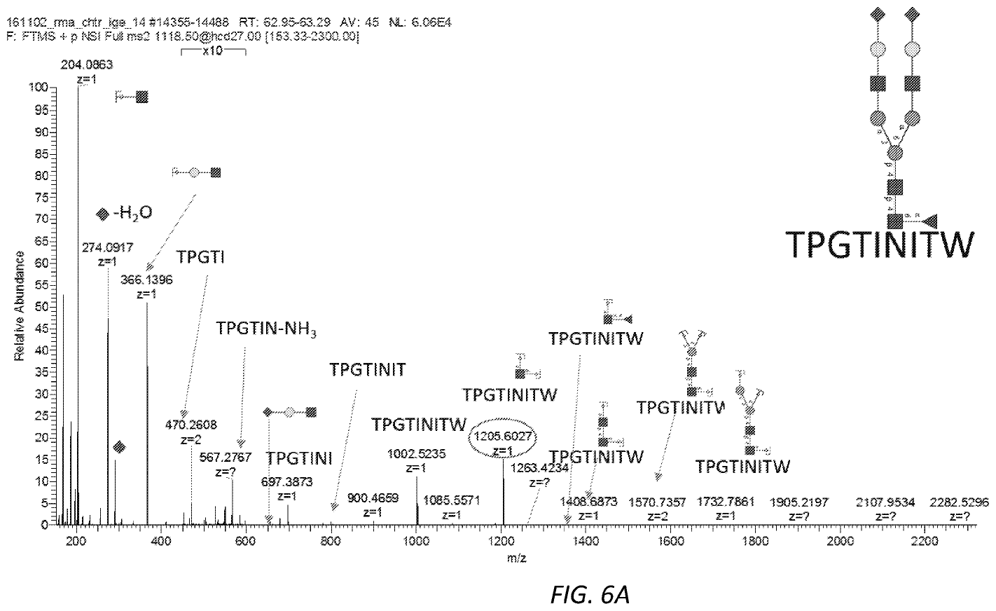

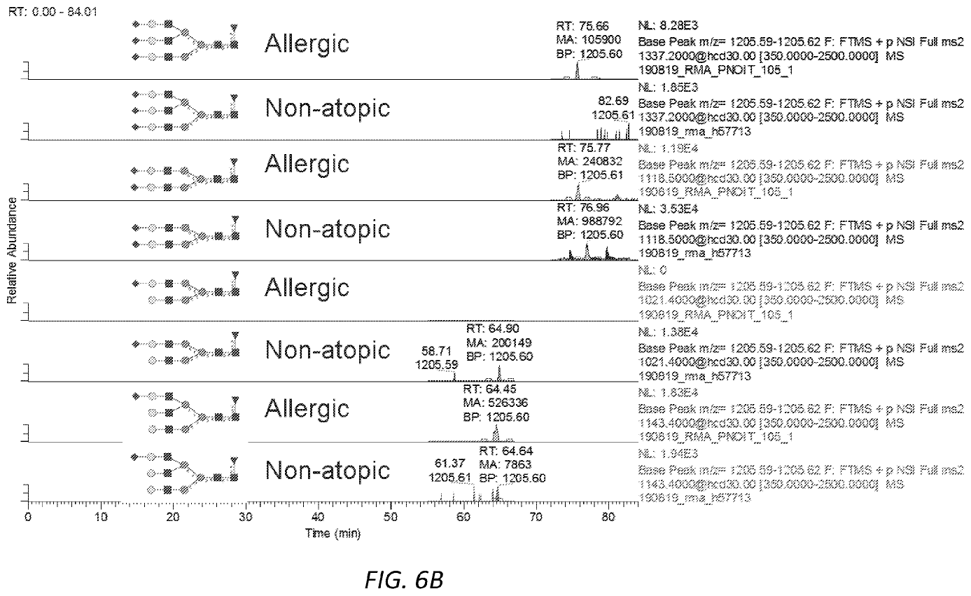

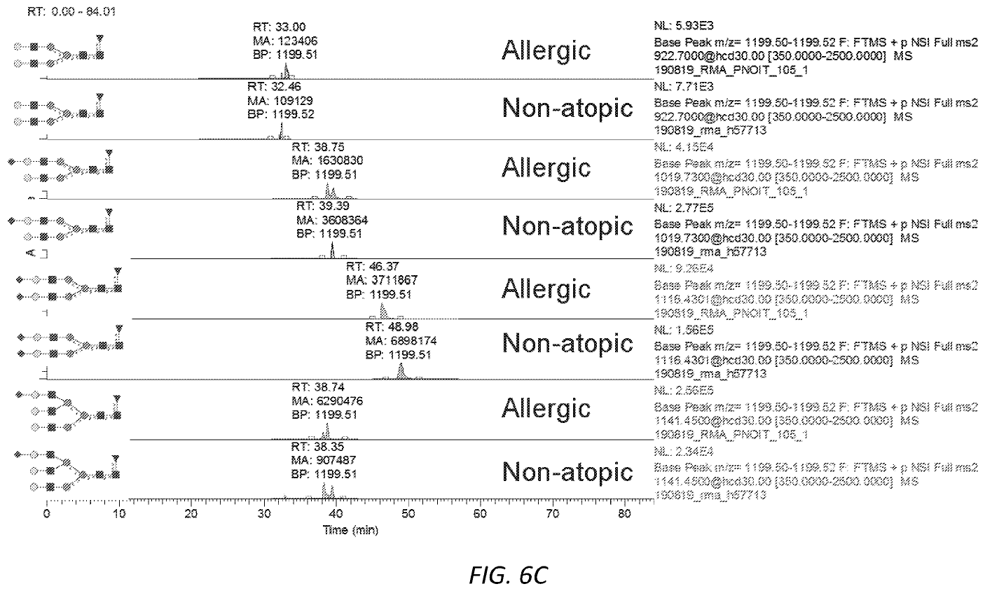

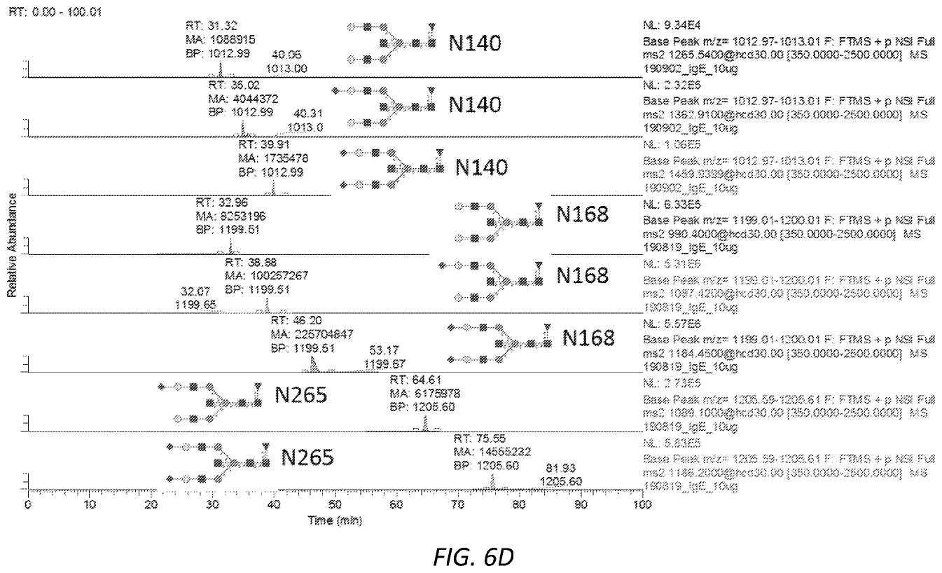

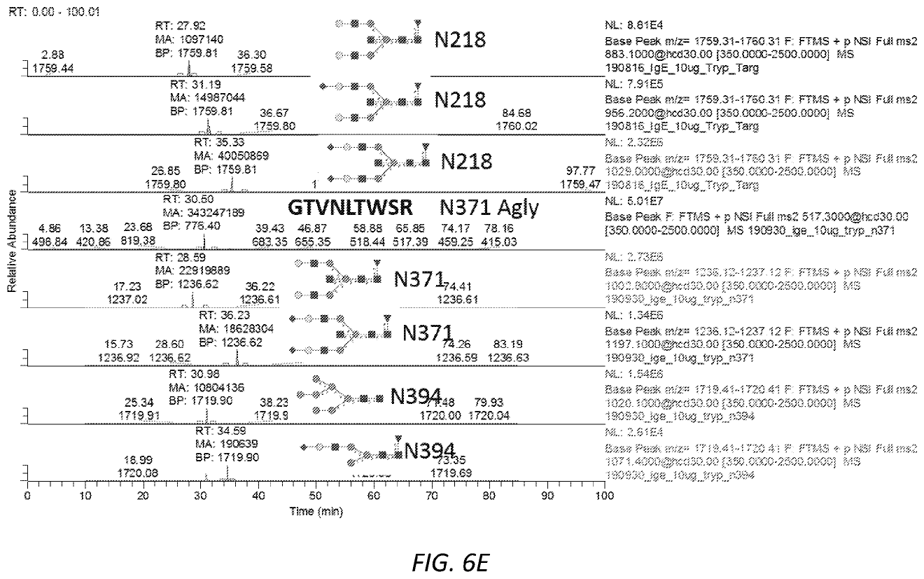

The isolated polyclonal primary hIgE and myeloma hIgE (Sigma Aldrich AG30P) was prepared for proteolysis by denaturing the protein in 6M guanidine HCl followed by reduction with dithiothreitol and alkylation with iodoacetamide followed by dialysis into 25 mM ammonium bicarbonate pH 7.8. Proteolysis was done with either trypsin to quantify N218, N371 and N394 or chymotrypsin to quantify N140, N168 and N265. For the tryptic digest IgE was incubated with trypsin (Trypsin Gold Promega) at a 1:50 enzyme to substrate ratio overnight at 37 C. For the chymotryptic digest IgE was incubated with chymotrypsin (Sequencing Grade Promega) at a 1:100 enzyme to substrate ratio for 4 hours at 25 C. Both enzymes were quenched with formic acid added to 2% w/w. The separation was performed on a Thermo EasySpray C18 nLC column 0.75 um×50 cm using water and acetonitrile with 0.1% formic acid for mobile phase A and mobile phase B respectively. A linear gradient from 1% to 35% mobile phase B was run over 75 minutes. Mass spectra were recorded on a Thermo Q Exactive mass spectrometer operated in positive mode using data independent acquisition (DIA) targeting the masses shown. Glycopeptides were quantified based on the extracted ion area of the Y1 ion (FIGS. 6A-E). The relative abundance was calculated for all identified glycan species for each site. Myeloma IgE (Sigma Aldrich AG30P) was run prior to paired sample sets to monitor retention time shifts and ensure consistency in the analytical results across the sample set. The percentage of glycan moieties at each site was calculated using the relative abundance of each glycan. For example, if a particular site was determined to have 60% monosialylated, fucosylated glycans (A1F), and 40% of disialylated, fucosylated glycans (A2F), the number of sialic acids at one site would be 1.4 (0.6×1+0.4×2), and total 2.8 sialic acids per molecule accounting for two sites.

### Mice

Five- to six-week-old female BALB/c mice were purchased from the Jackson Laboratory and used in these studies. All mice were housed in specific pathogen-free conditions according to the National Institutes of Health (NTH), and all animal experiments were conducted under protocols approved by the MGH IACUC. For all experiments, age- and sex-matched mice were randomized allocating to experimental group, with 4-5 mice per group, and repeated at least three independent times.

Passive Cutaneous Anaphylaxis (PCA) was conducted as previously described²³. In brief, monoclonal ^{Sia}mIgE or ^{As}mIgE specifically for OVA or dinitrophenyl (DNP, clone SPE-7; Sigma-Aldrich) was injected intradermally in the mice ears. For experiments where OVA-specific ^{As}mIgE was added to OVA-specific ^{Sia}mIgE, a mIgE isotype control (clone MEA-36, Biolegend) was included. The next day mice were intravenously challenged with PBS containing 125 μg OVA (Sigma-Aldrich) or DNP-Human Serum Albumin (DNP-HSA; Sigma-Aldrich) and 2% Evans blue dye in PBS. 45 min after challenge, the ears were excised and minced before incubation in N,N-dimethyl-formamide (EMD Millipore) at 55° C. for 3 h. The degree of blue dye in the ears was quantitated by the absorbance at 595 nm.

Passive Systemic Anaphylaxis (PSA) was elicited as previously described with minor modifications^{42,43}. Briefly, mice were injected intravenously with monoclonal mIgE specific for OVA or DNP (clone SPE-7; Sigma-Aldrich) in PBS and challenged the next day intravenously with PBS containing 1 mg OVA (Sigma-Aldrich) or DNP-HSA (Sigma-Aldrich). For examining the therapeutic potential of ^{As}mIgE, mice that had been injected intravenously with 10 μg DNP-specific mIgE (clone SPE-7; Sigma-Aldrich) the first day were injected intravenously with PBS, 20 μg OVA-specific ^{Sia}mIgE or 20 μg OVA-specific ^{As}mIgE the next day and challenged with 1 mg DNP-HSA or OVA (Sigma-Aldrich) the third day. For testing the therapeutic potential of NEU^{Fcε}, mice injected intravenously with 10 μg OVA-specific mIgE the first day were further injected intravenously with PBS, 100 μg NEU^{Fcε} or 100 μg mIgE isotype control (clone MEA-36, Biolegend) the next day and challenged with 1 mg OVA (Sigma-Aldrich) the third day. Core temperature was recorded at the baseline and every 10 min after the allergen challenge by a rectal microprobe thermometer (Physitemp). Histamine in the blood was quantified by histamine enzyme immunoassay kit (SPI-Bio) according to the manufacturer's protocol. Briefly, histamine in the blood was derivatized and incubated with plate precoated with monoclonal anti-histamine antibodies and histamine-AchE tracer at 4° C. for 24 h. The plate was then washed and developed with Ellman's reagent and the absorbance measured at 405 nm.

Passive Food Anaphylaxis (PFA) was elicited by adapting PSA described above. Briefly, mice injected intravenously with 20 μg monoclonal mIgE specific for TNP (clone MEA-36; Biolegend) in PBS the first day were administered with 20 mg TNP-OVA in PBS (Biosearch Technologies) by oral gavage the next day. Core temperature was recorded at the baseline and every 10 min after the challenge by a rectal microprobe thermometer (Physitemp).

To determine in vivo half-lives of ^{Sia}mIgE or ^{As}mIgE, mice were injected intraperitoneally with 30 μg DNP-specific ^{Sia}mIgE or ^{As}mIgE and the blood collected at the indicated times after injection into a Microtainer blood collection tube with clot actiator/SST gel (BD Diagonistics). The level of mIgE was quantified by mIgE ELISA described below.

### Human LAD2 Mast Cell Culture and Degranulation

Human LAD2 mast cell line was a generous gift of Dr. Metcalfe (MAID, NIH) and was maintained as previously described²³⁴⁴. Briefly, LAD2 cells were cultured in StemPro-34 SFM medium (Life Technologies) supplemented with 2 mM L-glutamine, 100 U/ml penicillin, 100 μg/ml streptomycin, and 100 ng/ml recombinant human stem cell factor (PeproTech). The cells were hemi-depleted each week with fresh medium and maintained at 2-5×10⁵ cells/ml at 37° C. and 5% CO2.

Degranulation assays were performed as previously described (Shade, 2015), LAD2 cells were sensitized overnight with 1 μg/mL OVA-specific hIgE at 1 μg/mL or 50 ng/mL peanut-allergic hIgE. The following day, the cells were pelleted by centrifugation, resuspended in HEPES buffer, plated in 96-well plates, and stimulated with allergen OVA or crude peanut extract at defined concentrations. Upon allergen challenge, mast cell degranulation was determined by the amount of substrate p-nitrophenyl N-acetyl-β-D-glucosamide digested by β-hexosaminidase release from the mast cell granules at the absorbance of 405 nm. To assess the effect of sialic acid removal on IgE-bound mast cells, IgE-sensitized LAD2 cells were treated with NEU^{Fcε}, heat-inactivated NEU^{Fcε}, mIgE isotype control (clone MEA-36, Biolegend) for 20 min before allergen challenge. To inactivate NEU^{Fcε}, the enzyme was heated at 95° C. for 10 min. To determine whether addition of a surrogate asialylated glycoprotein could recapitulate the phenotype of sialic acid removal from IgE, LAD2 cells sensitized with OVA-specific ^{Sia}hIgE were incubate with sialylated fetuin (^{Sia}Fetuin) or asialylated fetuin (^{As}Fetuin) at defined amount for 20 min before allergen challenge.

### Crude Peanut Extract Preparation

Unsalted dry-roasted peanuts (Blanched Jumbo Runner cultivar; Planters) were ground to a smooth paste, followed by washing with 20 volumes of cold acetone, filtered using Whatman paper, and dried as previously described²³. Protein was extracted by agitating the peanut flour overnight with PBS containing protease inhibitor cocktail without EDTA (Roche). The peanut protein extracts were collected as the supernatant after centrifugation at 24,000×g for 30 min.

### IgE Glycosylation Engineering

To remove sialic acids on IgE, IgE was digested with Glyko Sialidase A (Prozyme) at 37° C. for 72 h according to the manufacturer's instructions. To re-sialylate ^{As}mIgE by in vitro sialylation reaction, ^{As}mIgE was incubated with 5 mM Cytidine-5′-monophospho-N-acetylneuraminic acid (CMP-Neu5Ac2; Nacalai USA) in the sialylation buffer (150 mM NaCl, 20 mM HEPES, pH7.4) overnight at room temperature. Following reactions, OVA-specific ^{Sia}IgE or ^{As}IgE were purified by OVA-coupled beads to remove glycosylation modifying enzymes as described²³. All digestion or sialylation reactions were verified by lectin blotting or HPLC.

### Protein Gel Stain and Lectin Blotting

Equal amounts of ^{Sia}IgE or ^{As}IgE were resolved on 3-8% Tris-Acetate protein gels (Life Technologies) in SDS-PAGE under nonreducing conditions. For protein stain, gels were incubated in AcquaStain Protein Gel Stain (Bulldog Bio) for 1 h at room temperature and destained in distilled water. For lectin blotting, the protocol was conducted as described²³. Briefly, after resolved proteins on the gel were transferred to Immobilon-PSQ polyvinylidene difluoride membranes (Millipore Sigma), the membranes were blocked with 0.2% BSA in TBS for 1 hour at room temp, washed in TBS, followed by incubation with biotinylated Sambucus nigra lectin (SNA; 0.4 μg/ml; Vector Laboratories) in TBS with 0.1 M Ca²⁺ and 0.1 M Mg²⁺ for 1 hour at room temp to determine the level of terminal α2,6 sialic acids on N-linked glycans of proteins. The membrane was then washed in TBS and incubated with alkaline phosphatase conjugated goat anti-biotin (1:5000 dilution; Vector Laboratories) in TBS for 1 hour at room temp. Sialylated proteins on membranes were visualized by incubation with 1-Step NBT/BCIP plus Suppressor Substrate Solution.

### Basophil Activation Tests

Basophil activation was performed as previously described⁴⁵. Buffy coats of human blood from healthy, de-identified, consenting donors were obtained from the MGH Blood Transfusion Service. Peripheral blood mononuclear cells (PBMCs) were separated from buffy coats by a density gradient centrifugation using Ficoll Paque Plus (GE Healthcare) and resuspended in 0.5% BSA in RPMI 1640 Media (GE Healthcare). PBMCs were incubated for 2 min with ice-cold lactic acid buffer (13.4 mM lactate, 140 mM NaCl, 5 mM KCl, pH 3.9) to remove endogenous human IgE on the cell surface prior to neutralization by 12% Tris (pH 8). Cells were then washed and incubated 1 hour at 37° C. with 1 μg OVA-specific ^{Sia}hIgE or ^{As}hIgE per 1×10⁶ cells in basophil activation buffer (0.5% BSA, 2 mM CaCl₂ and 2 mM MgCl₂ in RPMI 1640 Media). Sensitized cells were washed and resuspended in basophil activation buffer supplemented with 10 ng/mL human IL3 (Peprotech) prior to 30 min OVA activation. Activation was stopped by addition of ice-cold 0.2 M EDTA in FACS buffer. Activated cells were washed and resuspended in FACS buffer to proceed antibody staining for basophil activation markers.

### Flow Cytometry

Antibodies used for surface allergen staining are listed in Table A. For staining for mouse cells, suspension cells were incubated with anti-mouse CD16/CD32 (clone 2.4G2, BD Biosciences) prior to antibody staining. Cells were incubated in FACS buffer with desired staining antibodies for 20 minutes at 4° C. Cells were then washed in FACS buffer before being acquired by an LSRII flow cytometer (BD Biosciences) or CytoFLEX (Beckman Coulter). Data were analyzed using FlowJo software version 10.4 software (Tree Star). To quantify hIgE loading following sensitization, PBS or 1 μg/mL OVA-specific ^{Sia}hIgE or ^{As}hIgE were incubated with 2.5×10⁵ cells/mL LAD2 mast cells overnight before wash with FACS buffer and stained with OVA-A647. To quantify dermal mast cell IgE loading, single cell suspensions were generated from mouse ears as described²³. Ears were intradermally injection of 40 ng OVA-specific ^{Sia}mIgE or ^{As}mIgE. The following day, ears were removed, separated into dorsal and ventral halves, and minced before incubation in DMEM containing 2% FCS, 1% HEPES, 500 u/mL collagenase type 4 (Worthington), 0.5 mg/mL hyaluronidase (Sigma) and Dnase I (Roche) at 37° C. for 1 h at 180 RPM. The digested sample was then subjected to disruption by Gentle MACS and filtered through a 70 μm cell strainer followed by a 40 μm cell strainer in FACS buffer (2 mM EDTA and 0.5% Bovine Serum Albumins (BSA) in PBS).

TABLE A
Antibodies used for in various experiments

| FACS assay | Target | Clone | Fluorochromes | Vendor | Dilution |
|---|---|---|---|---|---|
| IgE loading on | Mouse CD45 | 30-F11 | APC/Cyanine7 | Biolegend | 1:400 |
| mouse skin mast | Mouse/human | M1/70 | PerCP/Cyanine5.5 | Biolegend | 1:100 |
| cells | CD11b | | | | |
| | Mouse CD11c | N418 | FITC | Biolegend | 1:100 |
| | Mouse Ly- | RB6-8C5 | FITC | Biolegend | 1:100 |
| | 6G/Ly-6C | | | | |
| | (Gr-1) | 2B8 | APC | Biolegend | 1:100 |
| | Mouse CD117 | | | | |
| | (c-Kit) | | | | |
| | Mouse IgE | RME-1 | PE | Biolegend | 1:100 |
| IgE loading on | Human CD117 | 104D2 | PE | Biolegend | 1:400 |
| human LAD2 cells | (c-kit) | | | | 1:400 of 2 |
| | Ovalbumin | | A647 | Invitrogen | mg/mL stock |
| | Human IgE | MHE-18 | APC | Biolegend | 1:400 |
| Basophil activation | Human HLA- | L243 | PE/Cy7 | Biolegend | 1:35 |
| tests | DR | | | | |
| | Human CD123 | 6H6 | PE | Biolegend | 1:30 |
| | Human CD63 | H5C6 | FITC | Biolegend | 1:30 |
| General | Viability | | eFluor 450 | eBioscience | 1:500 |

| ELISA assay | Target | Catalog # | Conjugation | Vendor | Dilution |
|---|---|---|---|---|---|
| Mouse IgE ELISA | mIgE | A90-115A | No | Bethyl | 1:200 |
| | mIgE | A90-115P | HRP | Bethyl | 1:30,000 |
| Human IgE ELISA | hIgE | A80-108A | No | Bethyl | 1:200 |
| | hIgE | A80-108P | HRP | Bethyl | 1:30,000 |

| | | | Species/ | | |
|---|---|---|---|---|---|
| Immunoblotting | Target | Catalog # | conjugation | Vendor | Dilution |
| Immunoblotting for | Phospho-Syk | 2701 | Rabbit | Cell Signaling | 1:2000 |
| Syk | | | | Technology | |
| | Total | 2712 | Rabbit | Cell Signaling | 1:2000 |
| | | | | Technology | |
| Secondary antibody | Rabbit IgG | W4011 | HRP | Promega | 1:500 |
| Immunoblotting for | Actin | sc-47778 | HRP | Santa Cruz | 1:50,000 |
| Actin | | HRP | | Biotechnology | |

| Passive | | | Antigen | |
|---|---|---|---|---|
| Anaphylaxis | Species | Clone | specificity | Vendor |
| IgE | Mouse | SPE-7 | dinitrophenyl | Sigma-Aldrich |
| | | | (DNP) | |
| IgE | Mouse | MEA-36 | Trinitrophenyl | Biolegend |
| | | | (TNP) | |

### Biolayer Interferometric Assays for Binding

Binding kinetics and affinity of protein interaction studies were performed by the Octet K2 system (Molecular Devices) using Octet buffer (PBS with 0.025% Tween and 1% BSA). For measuring hFcεRIα interaction, ligand 0.25 ng/mL His-tagged hFcεRIα (Acro Biosystems) was loaded onto Anti-Penta-HIS (HIS1K) Biosensors (Molecular Devices). For OVA interaction, ligand 100 ng/mL OVA was immobilized onto Amine Reactive Second-Generation (AR2G) Biosensors in 10 mM sodium acetate, pH 5 using EDC/Sulfo-NHS based chemistry. Association of analyte OVA-specific ^{Sia}hIgE or ^{As}hIgE was performed in 3-fold serial dilution from 90 to 1 nM or NEU^{Fcε} in 3-fold serial dilution from 24 to 0.3 nM in Octet buffer. Analyte dissociation was measured in Octet buffer. Analysis of binding kinetic parameters were performed by Octet data analysis software 10.0 using interaction of ligand-loaded biosensor with no analyte during association phase as the reference sensor.

### Immunoblotting for Syk Signaling

1.5×10⁶ LAD2 cells were sensitized with PBS or 1 μg/mL OVA-specific ^{Sia}hIgE or ^{As}hIgE. Sensitized cells were washed and resuspended in HEPES buffer the next day followed by OVA stimulation at 10 ng/mL OVA at 37° C. for indicated times. Cells were immediately centrifuged after OVA stimulation and the cell pellets lysed in ice-cold lysis buffer for 30 min on ice (RIPA buffer (Boston BioProducts), 1× Halt Protease Inhibitor Cocktail (Thermo Scientific), 1× Halt™ Phosphatase Inhibitor Cocktail (Thermo Scientific) and 2.5 mM EDTA). After incubation on ice, lysed pellets were passed rapidly through a 27 G needle on ice and centrifuged at maximal speed at 4° C. for 15 min to clear the membrane and nuclei. The protein concentration was quantified using Pierce BCA Protein Assay kit (Thermo Scientific) and 20 μg of protein lysate was loaded per well on 4-12% Bis-Tris protein gels (Life Technologies) in SDS-PAGE under denaturing and reducing conditions. Briefly, after protein transferred to PVDF membranes described as above, the membranes were blocked with 5% milk in TBS with 0.1% Tween (TBST) for 1 hour at room temp, washed in TBST, followed by incubation with 1:2000 Rabbit anti-Phospho-Syk (Tyr352) Antibody (Cell Signaling Techology) in 5% BSA in TBST overnight at 4° C. The membrane was then washed in TBST before incubating with anti-rabbit-HRP for 1 hour at room temp and washed in TBST again followed by chemiluminescent detection using Immobilon Western Chemiluminescent HRP Substrate (Millipore Sigma). To detect total Syk on the membrane, after chemiluminescent detection using autoradiography film, the membrane was stripped by incubating in stripping buffer (2% SDS and 0.1 M β-mecaptoethanol in Tris buffer) at 50° C. for 30 min. The stripped membranes were then blocked, washed as above and then incubated with 1:2000 Rabbit anti-Syk Antibody (Cell Signaling Techology) for 2 h in 5% BSA in TBST at room temp before incubating with 1:30,000 anti-rabbit-HRP for 1 hour at room temp. To probe for β-Actin, the stripped membranes were incubated with 1:150,000 anti-β-Actin HRP (Santa Cruz Biotechnology) for 1 hour at room temp, washed and signal determined by chemiluminescent detection.

### Calcium Flux

5×10⁵ LAD2 cells were sensitized overnight with PBS or 500 ng/mL OVA-specific ^{Sia}hIgE or ^{As}hIgE. Next day, sensitized cells were washed before loading with 2 μM Fluo-4-AM (Invitrogen) at 37° C. in HEPES buffer for 20 minutes. After loading, the cells were washed and resuspended in HEPES buffer. Fluorescence was filtered through the 530/30 band pass filter and collected in FL-1/FITC. Baseline Ca²⁺ fluorescence levels were recorded for 1 minute on the Accuri C6 (BD Biosciences) before the addition of indicated allergen or buffer to each sample. At the end of allergen stimulation, cells were added 2 μM Ca²⁺ ionophore A23187 (Sigma) as a positive control.

### Generation of NEU^{Fcε}

The neuraminidase fusion protein was designed by fusing a kappa light chain secretion signal sequence and the sialidase gene from Arthrobacter urefaciens (EC 3.2.1.18, gene AU104)⁴⁶. Stop codon of the AU104 was omitted, instead, a short flexible linker peptide (GGGGGG), mouse IgE Cε2, Cε3, Cε4, and His6-tag was inserted to the C-terminus of the sialidase. The gene was codon optimized for human and synthesized by GenScript. The protein of 288 kDa was then produced by WuXi biologics. Sialidase activity of NEU^{Fcε} was determined by the level of p-nitrophenol released from 250 μM 2-O-(p-Nitrophenyl)-α-D-N-acetylneuraminic acid (Sigma) in 100 mM sodium phosphate (pH 5.5) for 10 min at 37° C. The reaction was terminated by adding 0.5 M sodium carbonate and the absorbance quantified at 405 nm.

### Statistical Analyses

All statistical analyses were performed using Prism 8 (GraphPad Software), and results are shown as means with SEM. Un-paired and paired Student's t test were used for parametric comparisons of two unmatched and matched groups, respectively. For comparisons of two sample groups of multiple non-parametric conditions, a two-way ANOVA with Sidak's multiple comparison test was used. For parametric comparison between three or more groups, one-way or two-way ANOVA with Tukey's multiple comparison test was used. Accuracy of individual IgE glycan moieties capacity to distinguish allergic IgE was analyzed by receiver operating characteristic (ROC) curves. Area under each ROC curve (AUC) was calculated for each glycan moiety. AUC was interpreted as follows, where a maximum AUC of 1 indicates the specific glycan moiety is able to distinguish allergic IgE from non-allergic IgE. An AUC of 0.5 indicates the differentiation capacity of a specific glycan moiety is poor.

### Results

The present study examined whether allergic disease-specific glycosylation patterns existed for IgE, and if so, whether those patterns influenced IgE biological activity. Non-atopic adults reported no history of atopy, had low total IgE titers, and had little IgE reactivity to peanut allergen (Ara h 2), birch tree pollen allergen (Bet v 1), house dust mite allergen (Der p 1), or cat allergen (Fel d 1) (FIG. 1b, c, d; Table 1). Peanut allergic adults reported multiple atopies, had approximately two-fold higher total IgE titers, with IgE reactive to peanut allergen (Ara h 2) but not the other tested allergens, and were clinically diagnosed with peanut allergy as confirmed by oral peanut challenge (FIG. 1b, c, d; Table 1)⁹. We sensitized human LAD2 mast cells with similar amounts of total IgE enriched from the sera of these cohorts (FIG. 5a) and activated the cells by anti-IgE crosslinking. Intriguingly, less degranulation, as measured by β-hexosaminidase release, was observed in mast cells sensitized with IgE isolated from sera of non-atopic individuals compared peanut allergic patients (FIG. 5b), despite similar surface IgE loading (FIG. 5c, d). This suggested possible intrinsic functional differences between non-atopic and allergic IgE, independent of allergen specificity.

Next, the N-glycan residues on total IgE enriched from non-atopic and allergic serum was analyzed by mass spectrometry^{22,24,27}. This revealed similar numbers of mannose moieties between total non-atopic and allergic IgE (FIG. 1e). Fucose, N-acetyl glucosamine (GlcNAc), galactose, and sialic acid can be attached to complex glycans (FIG. 1a). While total fucose content was similar between non-atopic and allergic IgE (FIG. 1f), significantly increased levels of bisecting GlcNAc (biGlcNAc) and terminal galactose were found on non-atopic IgE (FIG. 1g, h) whereas increased terminal sialylation was detected on allergic IgE (FIG. 1i).

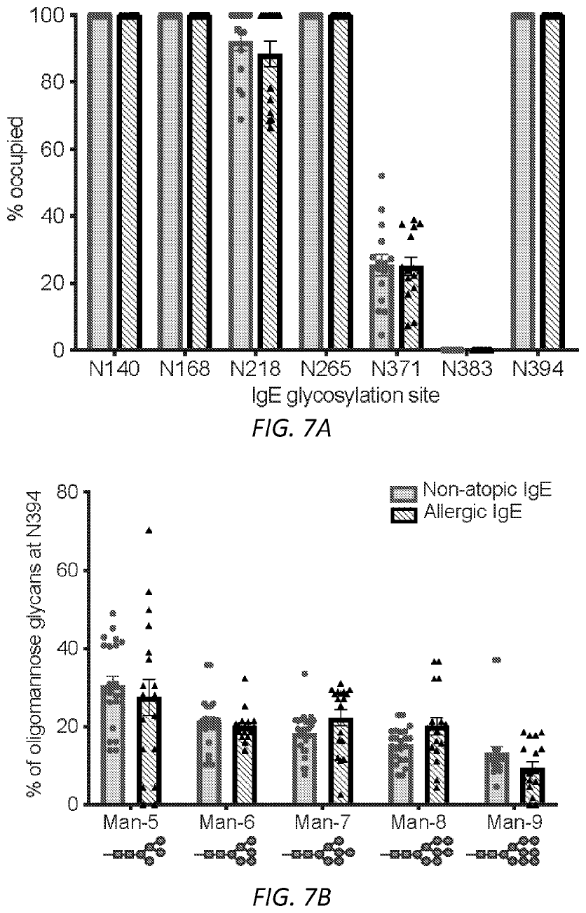

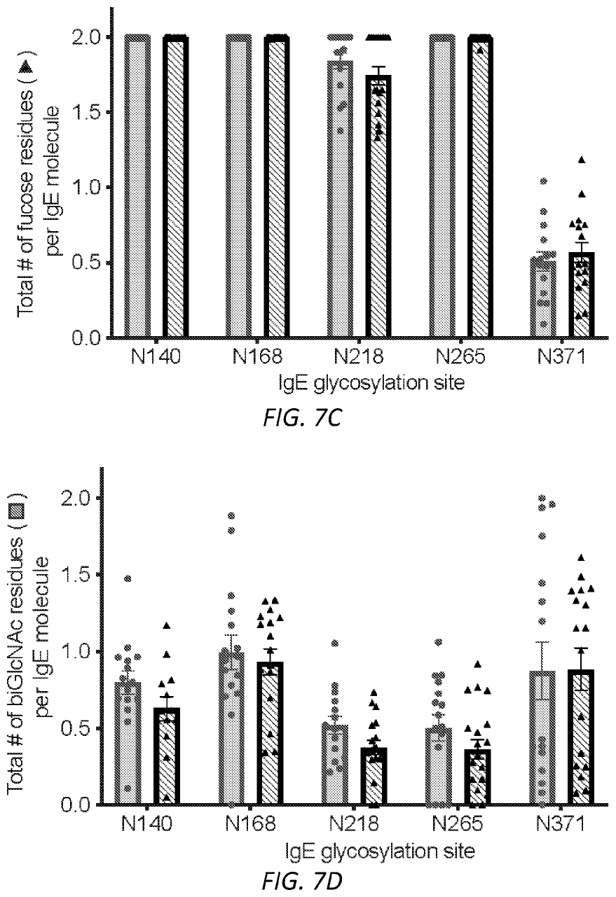

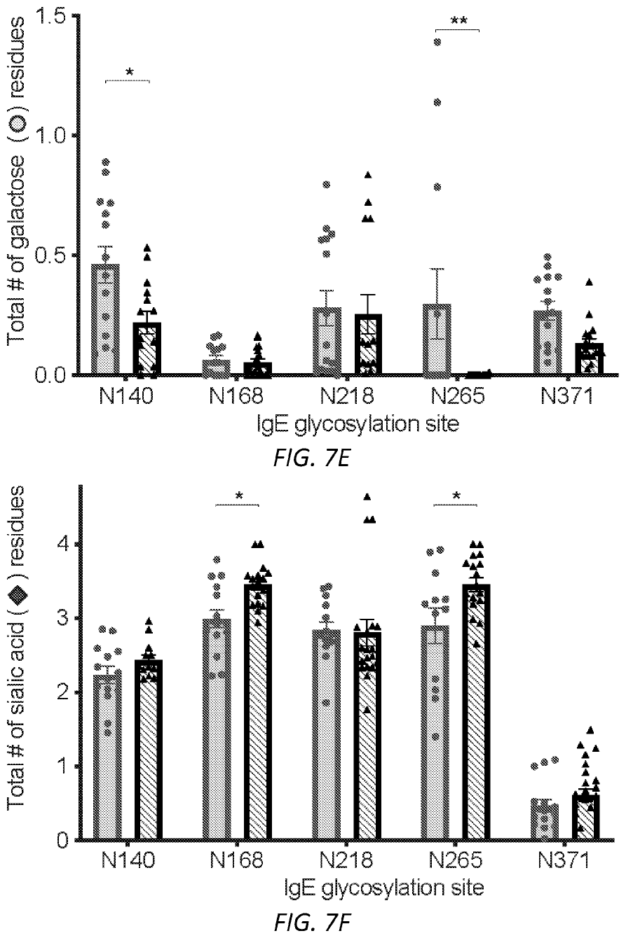

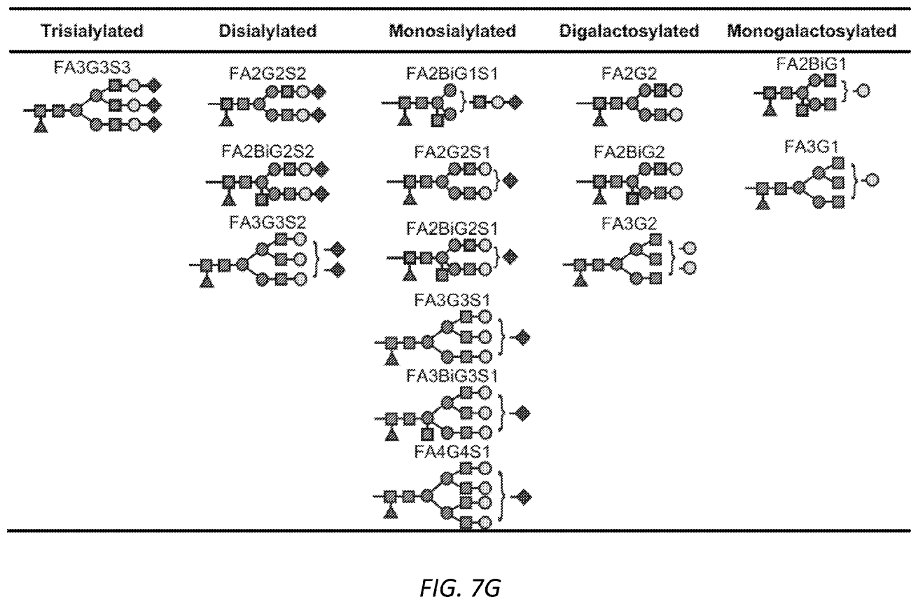

To determine whether the differences in glycan residues on total IgE were predictive of allergic disease, we assessed the variable glycan content on non-atopic and allergic IgE using Receiver Operating Characteristics (ROC) curves (FIG. 2a). The area-under these curves revealed that galactose and sialic acid content of IgE were strong predictors of allergic disease, but not fucose, mannose, nor biGlcNAc. Of note, differences in IgE sialylation were not sex- or age-dependent (FIG. 5e, f). We next asked where on the IgE molecule glycans differed between allergic and non-atopic individuals (FIGS. 6A-E, 7G; Tables 2-3). Site-specific analysis showed that N140, N168, N265 and N394 of IgE were fully occupied by N-linked glycans, with N218 and N371 partially occupied (75% and 30% respectively), and N383 completely unoccupied (FIG. 2b; FIG. 7a), consistent with previous results^{21,22,24}. The glycans at N394 were exclusively of oligomannose structure, with predominantly five mannose residues (Man-5) (FIG. 2b; FIG. 7b). N140, N168, N265, and N371 were occupied by complex, antennary structures. Fucose and biGlcNAc content was similar at all sites between samples (FIG. 7c, d). However, complex glycans terminating in galactose were enriched at N140 and N265 on non-atopic IgE, while terminal sialic acid moieties, particularly disialylated glycans were significantly enriched at N168 and N265 on allergic IgE (FIG. 2b; FIG. 7e-g). Together, these results reveal the specific glycosylation patterns that distinguish allergic from non-atopic total IgE.

TABLE 2
Targeted mass list for IgE glycosylation sites N140, N168
and N265 glycopeptides from the chymotryptic digest

| | CS | | Start | End | | |
|---|---|---|---|---|---|---|
| Mass [m/z] | [z] | Polarity | [min] | [min] | (N)CE | Comment |
| 1197.84000 | 3 | Positive | 23.00 | 30.00 | 27 | IgE N140 G2F |
| 1295.22000 | 3 | Positive | 28.00 | 34.00 | 27 | IgE N140 A1F |
| 1265.54000 | 3 | Positive | 25.00 | 33.00 | 27 | IgE N140 |
| | | | | | | G2F + BglcNAc |
| 1362.91000 | 3 | Positive | 29.00 | 34.00 | 27 | IgE N140 |
| | | | | | | A1F + BGlcNAc |
| 1391.91000 | 3 | Positive | 32.00 | 37.00 | 27 | IgE N140 A2F |
| 1459.94000 | 3 | Positive | 32.00 | 37.00 | 27 | IgE N140 |
| | | | | | | A2F + BglcNAc |
| 1416.94000 | 3 | Positive | 28.00 | 34.00 | 27 | IgE N140 |
| | | | | | | A1F + LAcNAc |
| 1513.97000 | 3 | Positive | 32.00 | 37.00 | 27 | IgE N140 |
| | | | | | | A2F + LacNAc |
| 1319.57000 | 3 | Positive | 23.00 | 30.00 | 27 | IgE N140 |
| | | | | | | G2F + LacNAc |
| 922.70000 | | Positive | 30.00 | 34.00 | 27 | IgE N168 G2F |
| 990.40000 | 3 | Positive | 30.00 | 34.00 | 27 | IgE N168 |
| | | | | | | G2F + BGlcNAc |
| 1019.73000 | 3 | Positive | 36.00 | 40.00 | 27 | IgE N168 A1F |
| 1087.42000 | 3 | Positive | 36.00 | 40.00 | 27 | IgE N168 |
| | | | | | | A1F + BGlcNAc |
| 1184.45000 | 3 | Positive | 42.00 | 50.00 | 27 | IgE N168 |
| | | | | | | A2F + BGlcNAc |
| 1116.43000 | 3 | Positive | 42.00 | 50.00 | 27 | IgE N168 A2F |
| 1141.45000 | 3 | Positive | 36.00 | 40.00 | 27 | IgE N168 |
| | | | | | | A1F + LAcNAc |
| 1238.48000 | 3 | Positive | 42.00 | 50.00 | 27 | IgE N168 |
| | | | | | | A2F + LAcNAc |
| 1044.43000 | 3 | Positive | 30.00 | 34.00 | 27 | IgE N168 |
| | | | | | | G2F + LAcNAc |
| 501.80000 | 2 | Positive | 45.00 | 55.00 | 27 | IgE N265 Agly |
| 1337.20000 | 3 | Positive | 65.00 | 75.00 | 27 | IgE N265 A3F |
| 992.10000 | 3 | Positive | 35.00 | 55.00 | 27 | IgE N265 |
| | | | | | | G2F + GlcNAc |
| 1118.50000 | 3 | Positive | 60.00 | 65.00 | 27 | IgE N265 A2F |
| 1186.20000 | 3 | Positive | 60.00 | 65.00 | 27 | IgE N265 |
| | | | | | | A2F + GlcNAc |
| 1089.10000 | 3 | Positive | 52.00 | 58.00 | 27 | IgE N265 |
| | | | | | | A1F + GlcNAc |
| 924.40000 | 3 | Positive | 35.00 | 55.00 | 27 | IgE N265 G2F |
| 1021.40000 | 3 | Positive | 52.00 | 58.00 | 27 | IgE N265 A1F |
| 1143.40000 | 3 | Positive | 52.00 | 58.00 | 27 | IgE N265 |
| | | | | | | A1F + LacNAc |
| 1240.50000 | 3 | Positive | 60.00 | 65.00 | 27 | IgE N265 |
| | | | | | | A2F + LacNAc |

TABLE 3
Targeted mass list for IgE glycosylation N218, N371
and N394 glycopeptides from the tryptic digest

| Mass | CS | | Start | End | | |
|---|---|---|---|---|---|---|
| [m/z] | [z] | Polarity | [min] | [min] | (N)CE | Comment |
| 905.40000 | 4 | Positive | 27.00 | 32.00 | 27 | N218 A1F |
| 883.10000 | 4 | Positive | 24.00 | 30.00 | 27 | N218 G2F + |
| | | | | | | GlcNAc |
| 1231.50000 | 3 | Positive | 23.00 | 30.00 | 27 | N218 G3F |
| 519.60000 | 3 | Positive | 39.00 | 55.00 | 27 | Agly N218 |
| 1029.00000 | 4 | Positive | 48.00 | 63.00 | 27 | N218 A2F + |
| | | | | | | GlcNAc |
| 1142.20000 | 4 | Positive | 35.00 | 45.00 | 27 | N218 A3F |
| 1123.15000 | 3 | Positive | 24.00 | 30.00 | 27 | N218 G1F + |
| | | | | | | GlcNAc |
| 1069.10000 | 4 | Positive | 30.00 | 38.00 | 27 | N218 A2F + |
| | | | | | | LacNAc |
| 956.20000 | 4 | Positive | 27.00 | 33.00 | 27 | N218 A1F + |
| | | | | | | GlcNAc |
| 996.70000 | 4 | Positive | 27.00 | 33.00 | 27 | N218 A1F + |
| | | | | | | LacNAc |
| 1109.13000 | 3 | Positive | 24.00 | 30.00 | 27 | N218 G2F |
| 978.20000 | 4 | Positive | 30.00 | 38.00 | 27 | N218 A2F |
| 1002.80000 | 3 | Positive | 30.00 | 37.00 | 27 | N371 G2F + |
| | | | | | | GlcNAc |
| 1099.80000 | 3 | Positive | 35.00 | 40.00 | 27 | N371 A1F + |
| | | | | | | GlcNAc |
| 1197.10000 | 3 | Positive | 40.00 | 50.00 | 27 | N371 A2F + |
| | | | | | | GlcnAc |
| 948.80000 | 3 | Positive | 30.00 | 38.00 | 27 | N371 G1F + |
| | | | | | | GlcNAc |
| 1153.80000 | 3 | Positive | 35.00 | 40.00 | 27 | N371 A1F + |
| | | | | | | LacNAc |
| 1032.80000 | 3 | Positive | 34.00 | 39.00 | 27 | N371 A1F |
| 1045.80000 | 3 | Positive | 35.00 | 41.00 | 27 | N371 G1F + |
| | | | | | | GlcNAc + NeuAc |
| 1129.80000 | 3 | Positive | 40.00 | 50.00 | 27 | N371 A2F |
| 935.10000 | 3 | Positive | 30.00 | 35.00 | 27 | N371 G2F + |
| | | | | | | GlcNAc |
| 1056.80000 | 3 | Positive | 30.00 | 35.00 | 27 | N371 G3F |
| 517.30000 | 2 | Positive | 30.00 | 38.00 | 27 | N371 Agly |
| 912.10000 | 3 | Positive | 34.00 | 40.00 | 27 | N394 HM5 |
| 966.10000 | 3 | Positive | 34.00 | 40.00 | 27 | N394 HM6 |
| 1020.10000 | 3 | Positive | 34.00 | 40.00 | 27 | N394 HM7 |
| 1074.20000 | 3 | Positive | 34.00 | 40.00 | 27 | N394 HM8 |
| 1128.20000 | 3 | Positive | 34.00 | 40.00 | 27 | N394 HM9 |
| 1071.40000 | 3 | Positive | 38.00 | 45.00 | 27 | N394 A1F − |
| | | | | | | LAcNAc |
| 1179.50000 | 3 | Positive | 38.00 | 45.00 | 27 | N394 3, 6, 1, 1, 0 |
| 1130.50000 | 3 | Positive | 38.00 | 45.00 | 27 | N394 3, 6, 0, 1, 0 |

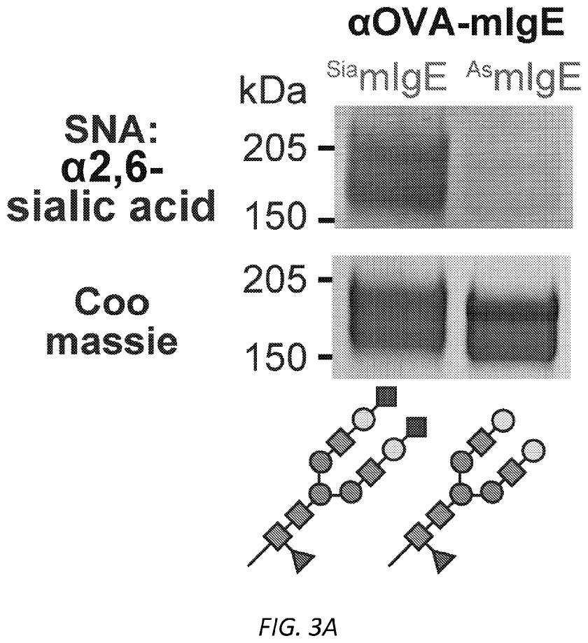

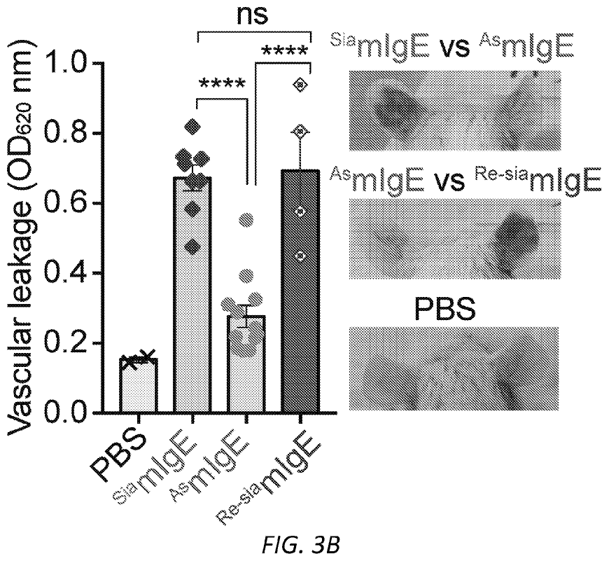

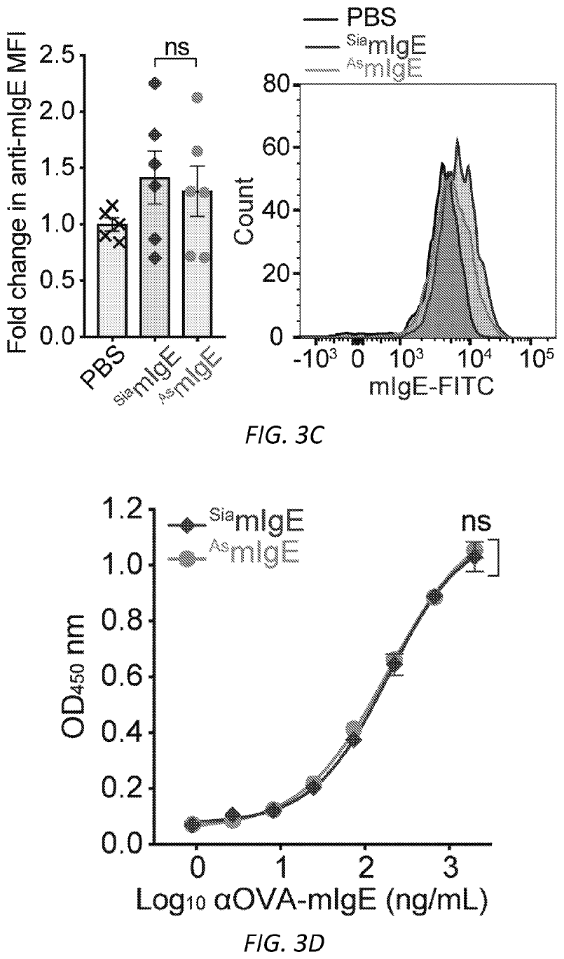

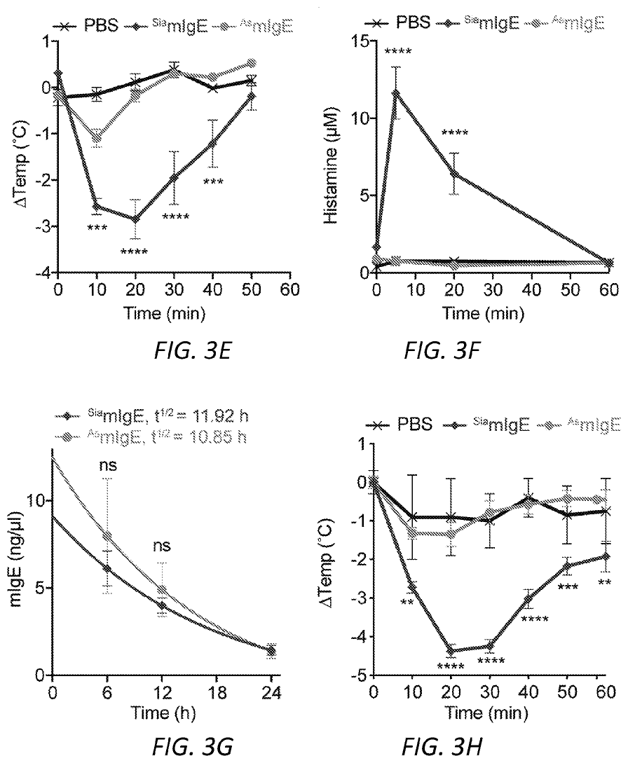

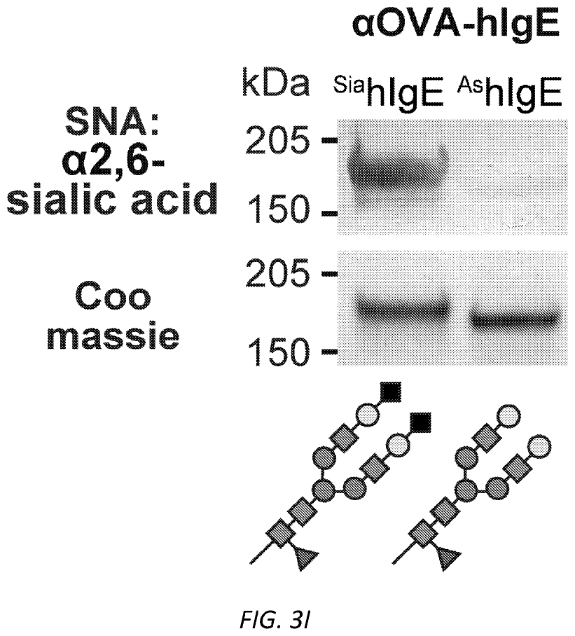

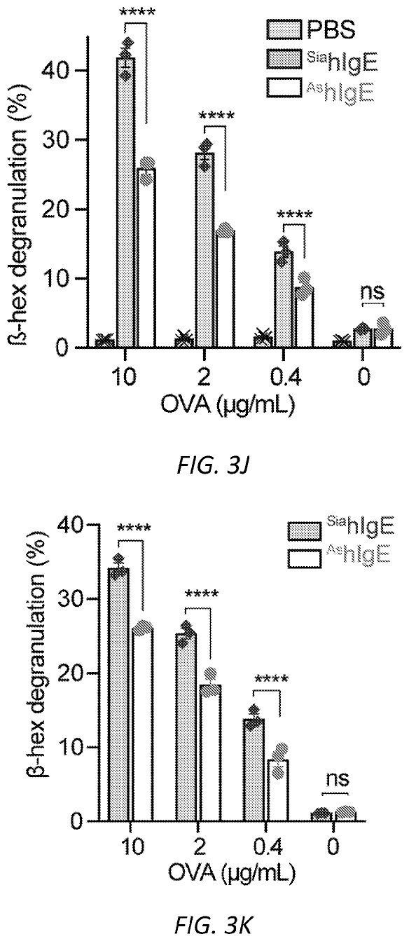

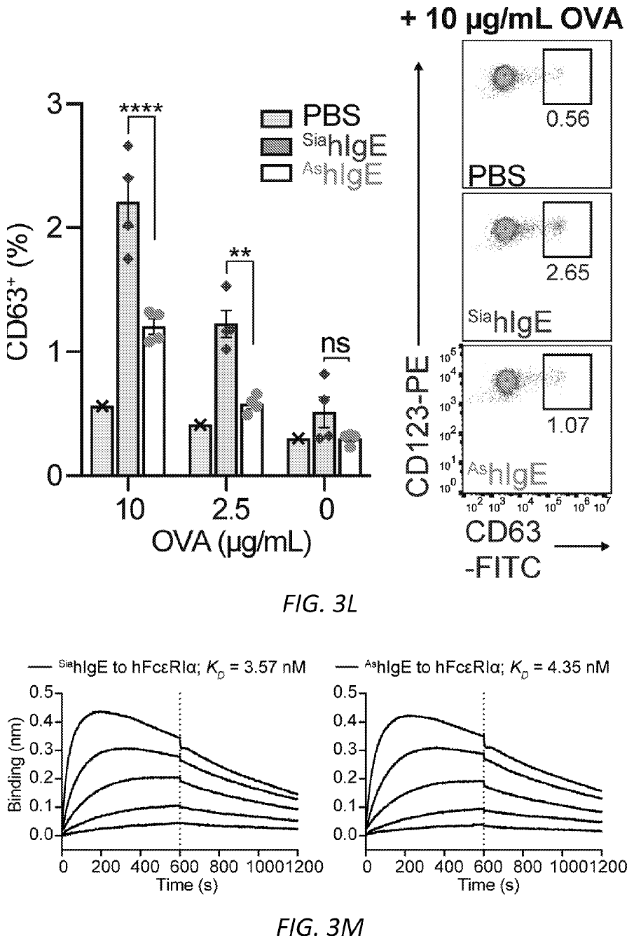

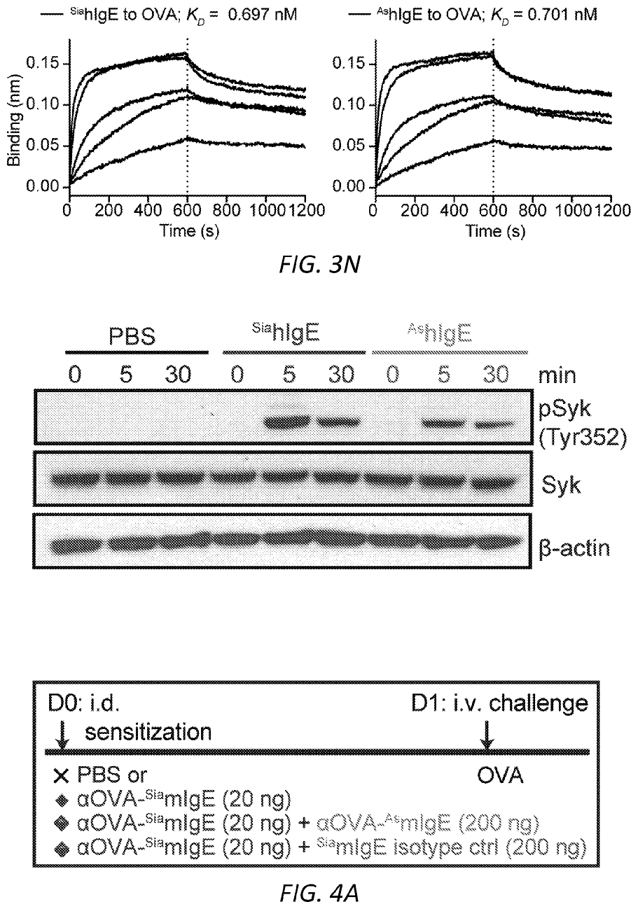

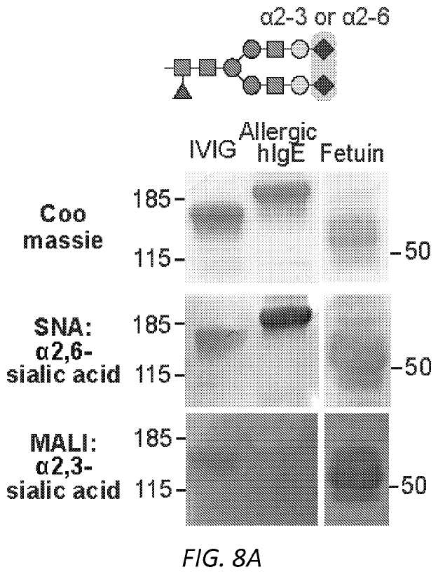

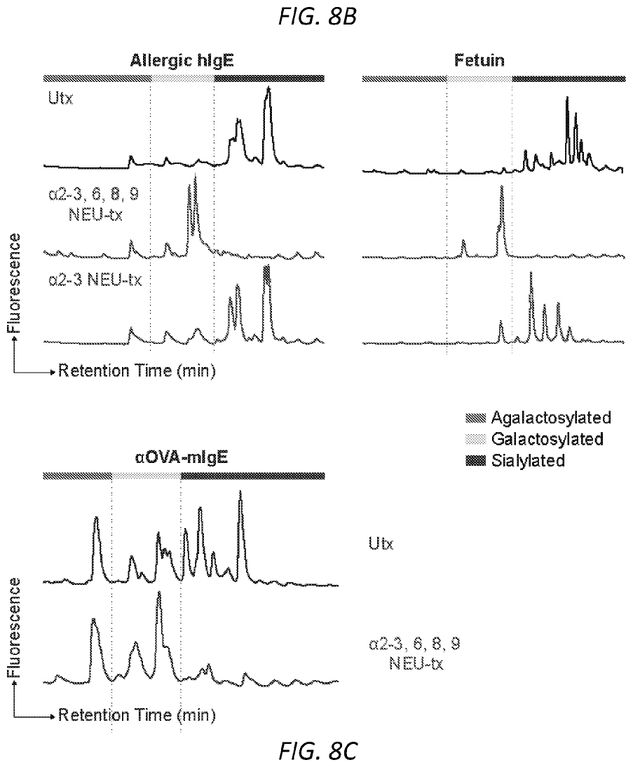

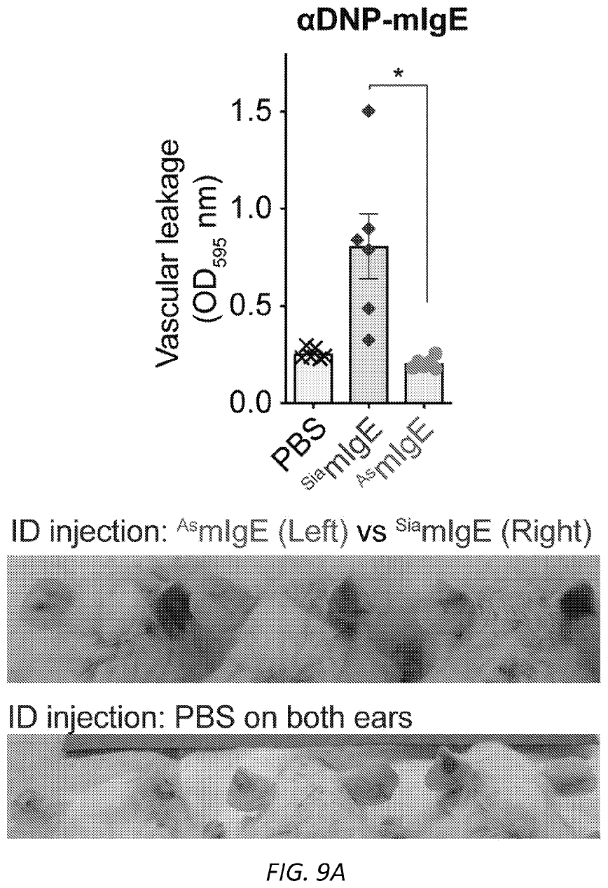

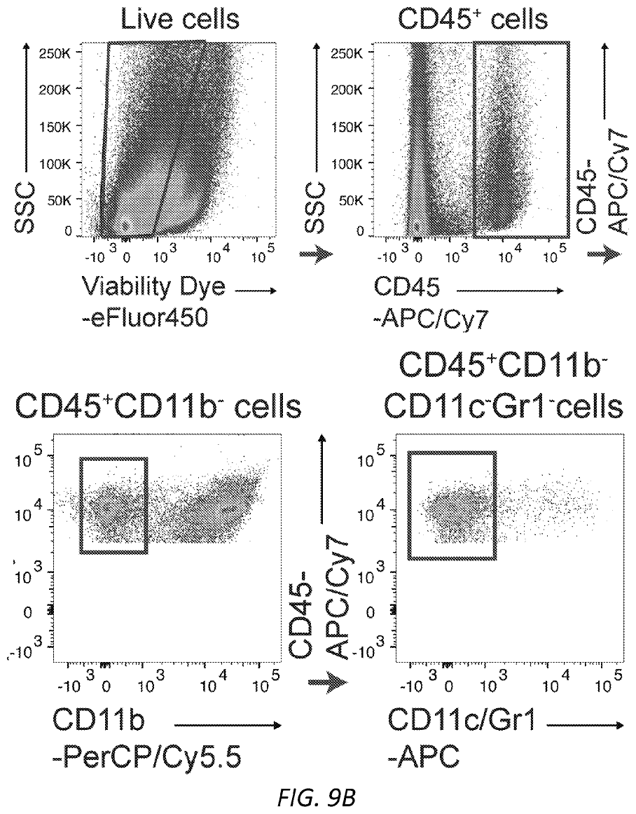

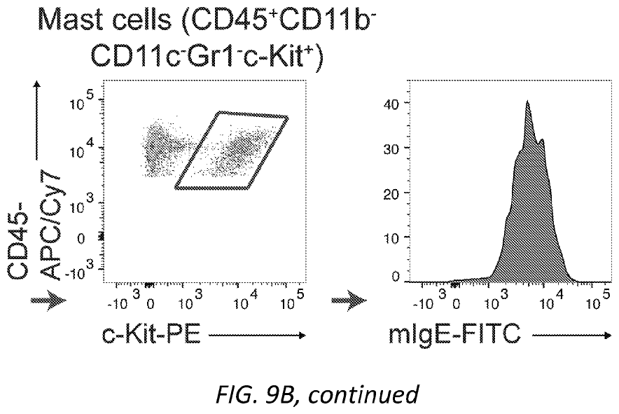

These findings raised the possibility that sialic acid content modifies IgE effector functions. Indeed, sialylation has been implicated in regulating most other antibody classes, including IgG1 anti-inflammatory activity, IgA neuropathy and influenza neutralization, and IgM-induced inhibitory signaling on B and T cells²⁸⁻³². However, the role for sialylation in modulating IgE function has not been described. Sialic acid was attached in α2,6 linkages on hIgE and mouse IgE (mIgE) as determined by neuraminidase (NEU) digestion assays and lectin blotting (FIG. 8, FIG. 3a), consistent with previous studies^{7,21,23}. Thus, we treated mIgE with NEU or digestion buffer as a control to generate mIgE of identical allergen-specificity differing only in sialic acid content (FIG. 3a). In a model of passive cutaneous anaphylaxis (PCA), mice were sensitized with PBS, OVA-specific sialylated-mIgE (^{Sia}mIgE), or OVA-specific asialylated-IgE (^{As}mIgE) intradermally in the ears. The next day, the mice were challenged with allergen, OVA, in Evan's blue dye intravenously. Forty minutes after challenge, the amount of blue dye in the ear was quantified as a surrogate of histamine-mediated vascular leakage. PBS-injection elicited little blue dye accumulation in the ear injection site, while significant blue coloration was observed in ^{Sia}mIgE-sensitized ears (FIG. 3b; FIG. 9a). Strikingly, ^{As}mIgE-sensitized ears exhibited markedly reduced blue coloration, indicative of attenuated anaphylaxis (FIG. 3b; FIG. 9a). To confirm sialic acid removal was responsible for reduced PCA reactivity, sialic acid was reattached to ^{As}mIgE by in vitro sialylation reaction (^{Re-sia}mIgE)³³. ^{Re-sia}mIgE, triggered a robust PCA reaction (FIG. 3b), demonstrating that IgE sialylation impacts the magnitude of anaphylaxis. Flow cytometry analysis of mast cells recovered from the mouse ears revealed no differences in IgE loading following sensitization with ^{Sia}mIgE or ^{As}mIgE (FIG. 3c; FIG. 9b) and ^{Sia}mIgE or ^{As}mIgE bound allergen similarly as determined by ELISA (FIG. 3d). Thus, attenuated local anaphylaxis by ^{As}mIgE was independent of IgE loading on mast cells in vivo or allergen recognition.

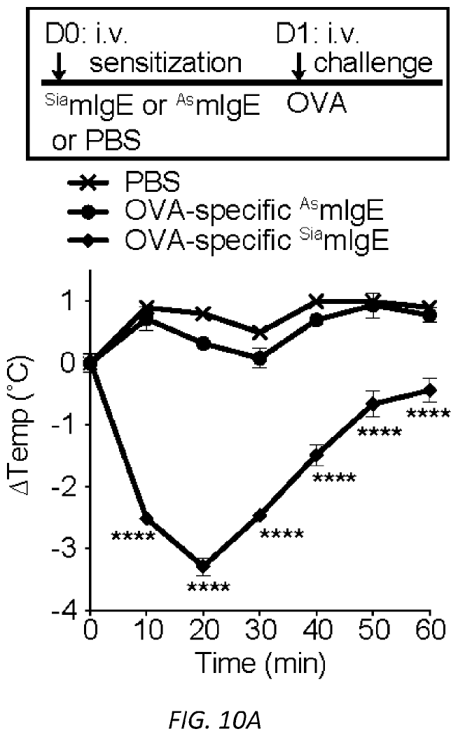

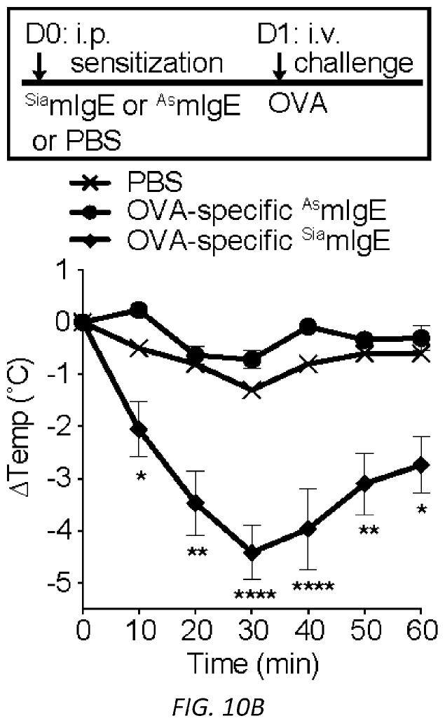

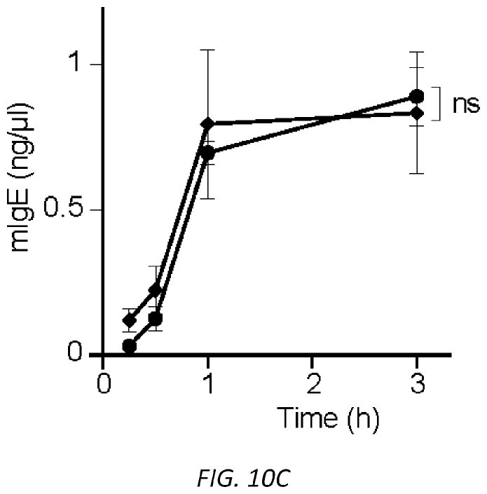

Next, we systemically sensitized mice with ^{Sia}mIgE, ^{As}mIgE, or PBS and challenged with allergen the following day in a model of passive systemic anaphylaxis (PSA). ^{Sia}mIgE-sensitized mice elicited a robust anaphylactic response underscored by 3° C. loss in temperature 20 minutes after allergen challenge (FIG. 3e; FIG. 10a, b). However, minimal temperature loss was observed in ^{As}mIgE- or PBS-sensitized mice (FIG. 3e; FIG. 10a, b). Consistently, a systemic increase in histamine was detected in ^{Sia}mIgE-sensitized animals following challenge, but not in ^{As}mIgE- or PBS-treated mice (FIG. 3f). Asialylated glycoproteins have decreased serum half-life³⁴, and we therefore compared the levels of ^{Sia}mIgE and ^{As}mIgE in circulation following systemic administration. However, sialic acid removal had little effect on IgE half-life (FIG. 3g, FIG. 10c). To extend these findings to a model of passive food allergy, we sensitized mice systemically with PBS, ^{Sia}mIgE or ^{As}mIgE, and challenged with allergen orally the following day. ^{Sia}mIgE sensitization, but not ^{As}mIgE- or PBS-sensitization resulted in a significant temperature loss following oral allergen challenge (FIG. 3h).

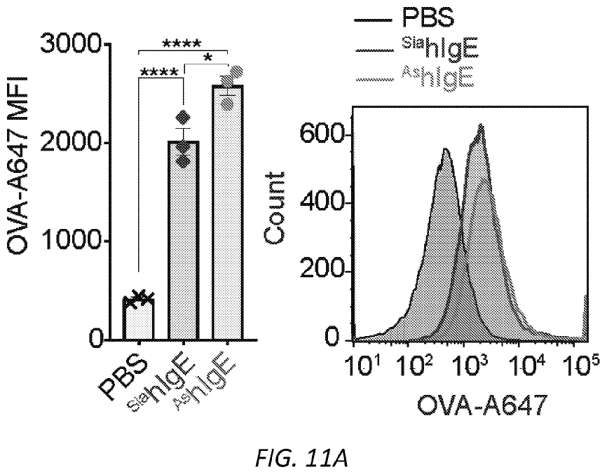

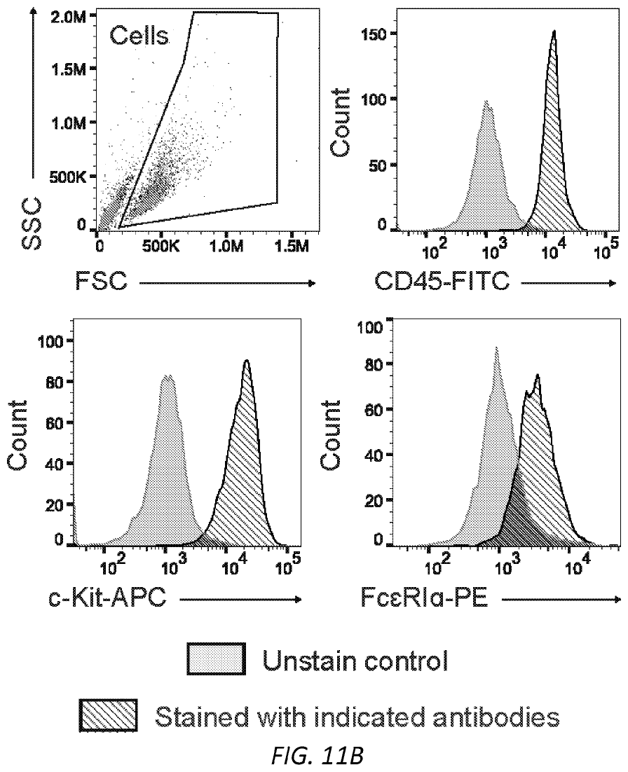

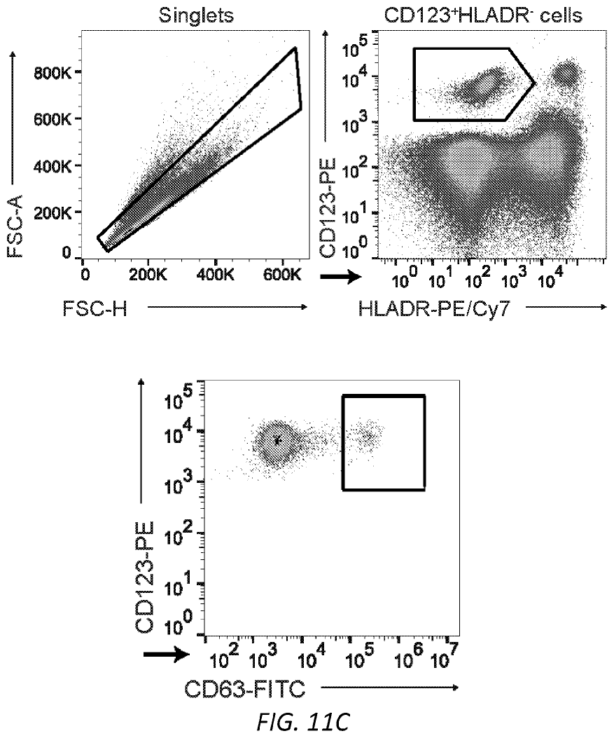

We next asked whether sialylation similarly regulated hIgE. We sensitized human LAD2 mast cells with PBS, sialylated or asialylated human IgE (^{Sia}hIgE and ^{As}hIgE, respectively, FIG. 3i). The cells were stimulated with allergen, and degranulation quantified by β-hexosaminidase release assays. ^{As}hIgE-sensitized cells had markedly reduced degranulation following allergen challenge, compared to ^{Sia}mIgE-sensitized cells (FIG. 3j). hIgE loading on LAD2 mast cells after sensitization was examined by flow cytometry and revealed comparable loading following ^{Sia}hIgE or ^{As}hIgE sensitization (FIG. 11a). Similar findings were observed in human mast cells derived from primary peripheral blood CD34⁺ cell culture, where ^{As}hIgE-sensitized cells had markedly reduced allergen-specific degranulation compared to ^{Sia}hIgE-sensitized cells (FIG. 3k; FIG. 11b). In parallel, primary basophils were sensitized with PBS, ^{Sia}hIgE and ^{As}hIgE and stimulated with allergen (FIG. 11c). ^{As}hIgE-sensitized basophils elicited reduced degranulation after allergen stimulation as measured by surface staining of the granule marker, CD63, compared to basophils sensitized with ^{Sia}hIgE (FIG. 3l). Although mast cell loading was similar between mouse and human ^{Sia}IgE and ^{As}IgE (FIG. 3c, FIG. 11a), we asked whether sialylation altered binding kinetics of hIgE to its receptor, FcεRI. Biolayer interferometry (BLI) assays revealed no difference in ^{Sia}hIgE and ^{As}hIgE interactions with FcεRI (FIG. 3m). Sialylation also did not alter IgE binding to the allergen (FIG. 3n). Thus, removing sialic acid from IgE attenuates its effector functions in vivo and in vitro, while binding to allergen, mast cells and FcεRI remained intact.

Because sialylation does not alter IgE interactions to allergen and receptor, we tested whether signaling downstream of FcεRI was affected by IgE sialylation. LAD2 mast cells sensitized with ^{Sia}hIgE or ^{As}hIgE were stimulated with allergen and cellular lysates collected at defined intervals. Western blotting of mast cell lysates for Syk revealed reduced phosphorylation at 5 and 30 minutes after stimulation (FIG. 4a). Similarly, calcium flux was reduced in ^{As}hIgE-sensitized LAD2 mast cells following allergen stimulation compared to ^{Sia}hIgE-sensitizated cells (FIG. 4b). We then asked whether a surrogate asialylated glycoprotein could recapitulate the phenotype of attenuating IgE by removing sialic acid. LAD2 mast cells were sensitized with ^{Sia}hIgE, and supplemented with either sialylated fetuin (^{Sia}Fetuin) or asialylated fetuin (^{As}Fetuin; FIG. 8b) during allergen stimulation. Quantifying the resulting degranulation revealed that addition of sialylated fetuin had no effect, while asialylated fetuin inhibited allergen-induced mast cell degranulation (FIG. 4c). Together, these results suggest that sialic acid removal exposes an inhibitory glycan that dampens FcεRI signaling.

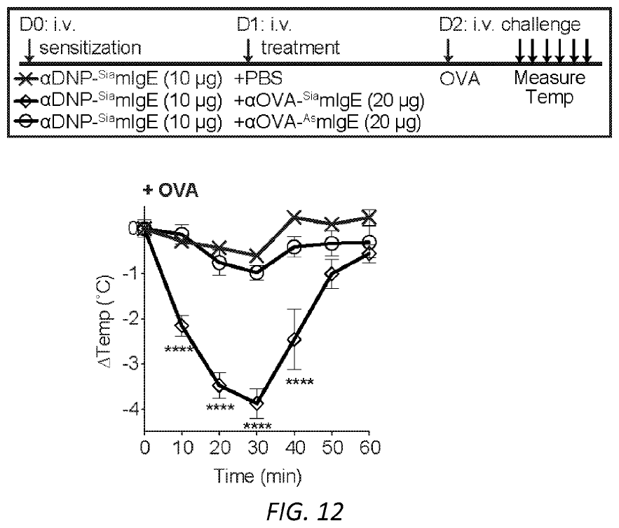

The observation that an asialylated glycoprotein could inhibit mast cell degranulation in vitro raised the possibly that ^{As}IgE could actively inhibit anaphylaxis in vivo. We therefore sensitized mice intradermally in the ears with PBS, OVA-specific ^{Sia}mIgE, a combination of OVA-specific ^{Sia}mIgE and ten-fold more OVA-specific ^{As}mIgE, or a combination of OVA-specific ^{Sia}mIgE and ten-fold more TNP-specific ^{Sia}mIgE isotype control. The next day mice were challenged with OVA and blue coloration of the ears quantified. Extensive vascular leakage occurred in ears sensitized with OVA-specific ^{Sia}mIgE alone (FIG. 4d). However, co-sensitization of OVA-specific ^{Sia}mIgE with either OVA-specific ^{As}mIgE or TNP-specific ^{Sia}mIgE both resulted in significantly reduced vascular leakage (FIG. 4d). Next, mice were systemically sensitized by DNP-specific ^{Sia}mIgE on day 0, and PBS, OVA-specific ^{Sia}mIgE, or OVA-specific ^{As}mIgE on day 1, and challenged with DNP-HSA on day 2. Intriguingly, mice that were sensitized with DNP-specific ^{Sia}mIgE on day 0 and PBS or OVA-specific ^{Sia}mIgE on day 1 exhibited robust temperature loss after allergen challenge. However, DNP-specific ^{Sia}mIgE-sensitized mice that received OVA-specific ^{As}mIgE on day 1 had significantly attenuated temperature loss upon allergen challenge (FIG. 4e). Systemic challenge of these treatment groups with OVA revealed that only mice sensitized with OVA-specific ^{Sia}mIgE resulted in temperature drop, while all other groups were unaffected (FIG. 12). These results suggest that ^{As}mIgE attenuates anaphylaxis by occupying FcεRI, but can actively dampen systemic anaphylaxis.

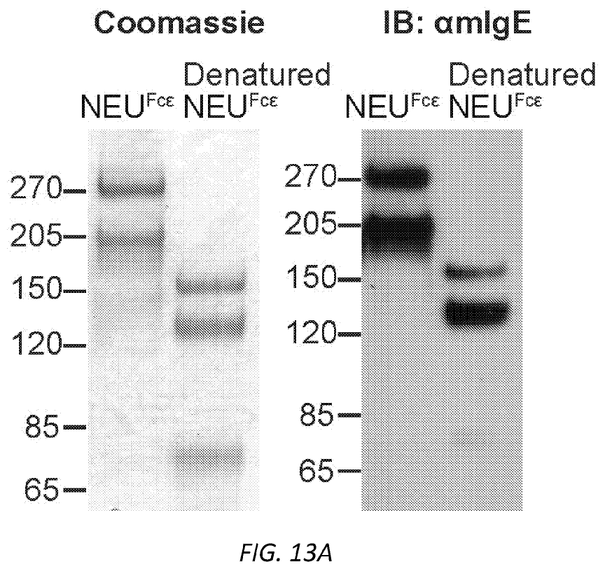

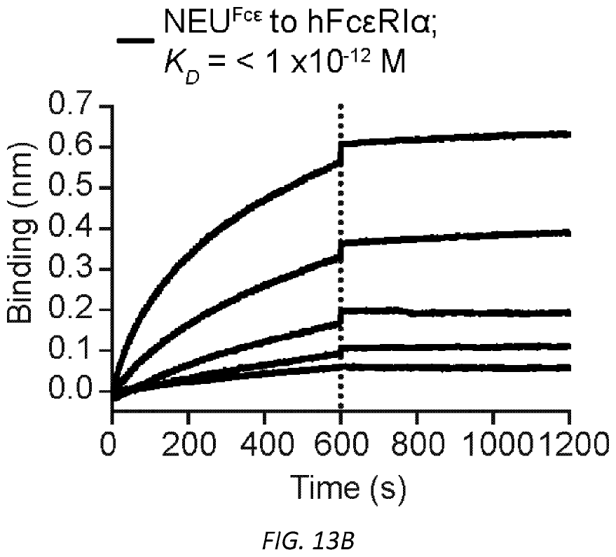

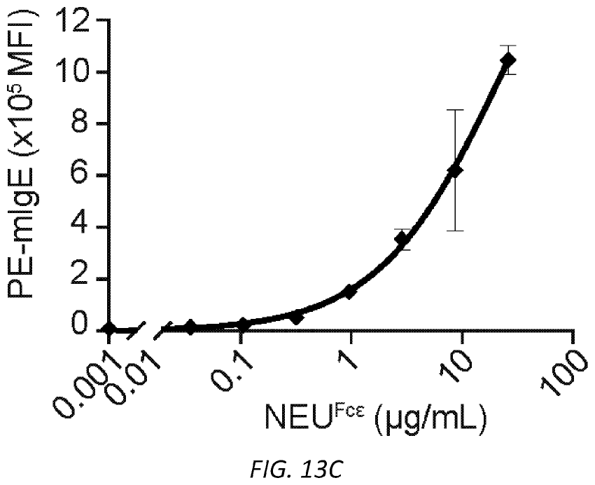

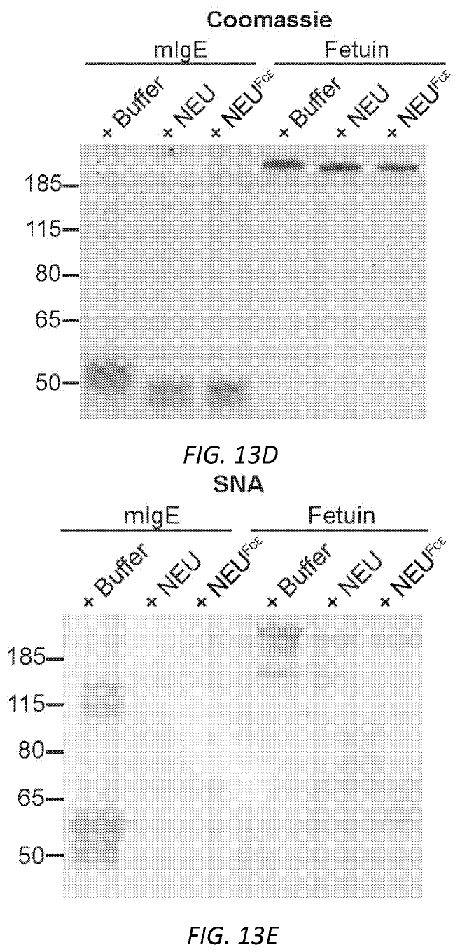

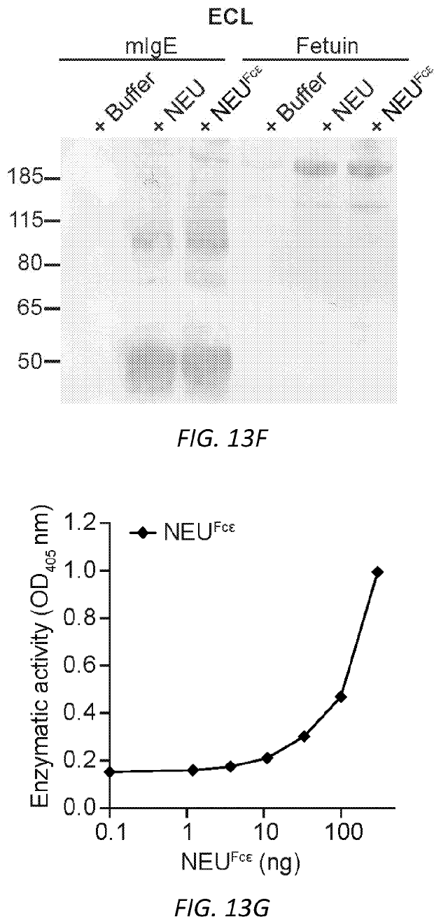

As sialic acid removal attenuated IgE effector functions, we explored whether targeting sialic acid on IgE-bearing cells is a viable strategy for attenuating allergic inflammation. Thus, we genetically fused a neuraminidase to the N-terminus of IgE Fc CE2-4 domains (Neu^{Fcε}, FIG. 4f; FIG. 13a) to direct sialic acid removal specifically to IgE-bearing cells. This fusion protein retained binding to FIERI in BLI binding assays (FIG. 13b), could be loaded on mast cells (FIG. 13c), and had neuraminidase activity (FIG. 13d-g). LAD2 mast cells were sensitized with OVA-specific ^{Sia}hIgE, and then incubated briefly with increasing concentrations of Neu^{Fcε}, heat-inactivated Neu^{Fcε}, or an IgE isotype to control for FIERI occupancy, and stimulated with OVA. Remarkably, treatment with Neu^{Fcε}, but not heat-inactivated Neu^{Fcε} nor the isotype control attenuated OVA-induced degranulation in a dose-dependent manner (FIG. 4g). To extend our findings to allergic hIgE from peanut allergic patients, we sensitized LAD2 mast cells with peanut allergic ^{Sia}hIgE and treated with Neu^{Fcε}, or an IgE isotype control. Consistently, allergen-induced degranulation was significantly attenuated by Neu treatment of peanut allergic ^{Sia}hIgE-sensitized cells compared to IgE isotype control treatment (FIG. 4h). Unsensitized LAD2 mast cells treated with Neu^{Fcε} did not degranulate (no IgE+Neu^{Fcε}, FIG. 4g, h), indicating Neu^{Fcε} treatment does not stimulate mast cells. We next explored the therapeutic potential of modulating sialic acid content in vivo. Mice were sensitized systemically with ^{Sia}mIgE on day 0, received PBS, Neu^{Fcε}, or IgE isotype control treatment on day 1. The following day, the mice were challenged systemically with allergen, and core body temperature measured as described above. ^{Sia}mIgE-sensitized mice that received PBS or isotype control exhibited robust drops in temperature (FIG. 4i). Remarkably, Neu^{Fcε} treatment significantly attenuated allergen-induced temperature drop, providing evidence of the therapeutic potential of targeting sialic acid on IgE-bearing cells.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1 Gupta, R. S. et at Prevalence and Severity of Food Allergies Among US Adults. JAMA Netw Open 2, e185630, doi:10.1001/jamanetworkopen.2018.5630 (2019).
2 Salo, P. M. et al. Prevalence of allergic sensitization in the United States: results from the National Health and Nutrition Examination Survey (NHANES) 2005-2006. The Journal of allergy and clinical immunology 134, 350-359, doi:10.1016/j.jaci.2013.12.1071 (2014).
3 Gould, H. J. & Sutton, B. J. IgE in allergy and asthma today. Nature reviews. Immunology 8, 205-217, doi:10.1038/nri2273 (2008).
4 Bird, J. A., Crain, M. & Varshney, P. Food allergen panel testing often results in misdiagnosis of food allergy. J Pediatr 166, 97-100, doi:10.1016/j.jpeds.2014.07.062 (2015).
5 Du Toit, G. et al. Randomized trial of peanut consumption in infants at risk for peanut allergy. The New England journal of medicine 372, 803-813, doi:10.1056/NEJMoa1414850 (2015).
6 Peters, R. L., Gurrin, L. C. & Allen, K. J. The predictive value of skin prick testing for challenge-proven food allergy: a systematic review. Pediatr Allergy Immunol 23, 347-352, doi:10.1111/j.1399-3038.2011.01237.x (2012).
7 Arnold, J. N., Wormald, M. R., Sim, R. B., Rudd, P. M. & Dwek, R. A. The impact of glycosylation on the biological function and structure of human immunoglobulins. Annual review of immunology 25, 21-50 (2007).
8 Begin, P. et al. Phase 1 results of safety and tolerability in a rush oral immunotherapy protocol to multiple foods using Omalizumab. Allergy, asthma, and clinical immunology: official journal of the Canadian Society of Allergy and Clinical Immunology 10, 7, doi:10.1186/1710-1492-10-7 (2014).
9 Patil, S. U. et al. Peanut oral immunotherapy transiently expands circulating Ara h 2-specific B cells with a homologous repertoire in unrelated subjects. The Journal of allergy and clinical immunology 136, 125-134 e112, doi:10.1016/j.jaci.2015.03.026 (2015).
10 Schneider, L. C. et al. A pilot study of omalizumab to facilitate rapid oral desensitization in high-risk peanut-allergic patients. The Journal of allergy and clinical immunology 132, 1368-1374, doi:10.1016/j.jaci.2013.09.046 (2013).
11 Oettgen, H. C. Fifty years later: Emerging functions of IgE antibodies in host defense, immune regulation, and allergic diseases. The Journal of allergy and clinical immunology 137, 1631-1645, doi:10.1016/j.jaci.2016.04.009 (2016).
12 Wang, T. T. et al. IgG antibodies to dengue enhanced for FcgammaRIIIA binding determine disease severity. Science 355, 395-398, doi:10.1126/science.aai8128 (2017).
13 Lu, L. L. et al. A Functional Role for Antibodies in Tuberculosis. Cell 167, 433-443 e414, doi:10.1016/j.cell.2016.08.072 (2016).
14 Wang, T. T. et al. Anti-HA Glycoforms Drive B Cell Affinity Selection and Determine Influenza Vaccine Efficacy. Cell 162, 160-169, doi:10.1016/j.cell.2015.06.026 (2015).
15 Parekh, R. B. et al. Association of rheumatoid arthritis and primary osteoarthritis with changes in the glycosylation pattern of total serum IgG. Nature 316, 452-457 (1985).
16 Espy, C. et al. Sialylation levels of anti-proteinase 3 antibodies are associated with the activity of granulomatosis with polyangiitis (Wegener's). Arthritis and rheumatism 63, 2105-2115, doi:10.1002/art.30362 (2011).
17 Wang, T. T. IgG Fc Glycosylation in Human Immunity. Curr Top Microbiol Immunol, doi:10.1007/82_2019_152 (2019).
18 Shields, R. L. et al. Lack of fucose on human IgG1 N-linked oligosaccharide improves binding to human Fcgamma RIII and antibody-dependent cellular toxicity. The Journal of biological chemistry 277, 26733-26740 (2002).
19 Kaneko, Y., Nimmerjahn, F. & Ravetch, J. V. Anti-inflammatory activity of immunoglobulin G resulting from Fc sialylation. Science 313, 670-673 (2006).
20 Washburn, N. et al. Controlled tetra-Fc sialylation of IVIg results in a drug candidate with consistent enhanced anti-inflammatory activity. Proceedings of the National Academy of Sciences of the United States of America 112, E1297-1306, doi:10.1073/pnas.1422481112 (2015).
21 Arnold, J. N. et al. The glycosylation of human serum IgD and IgE and the accessibility of identified oligomannose structures for interaction with mannan-binding lectin. Journal of immunology 173, 6831-6840 (2004).
22 Plomp, R. et al. Site-Specific N-Glycosylation Analysis of Human Immunoglobulin E. Journal of proteome research, doi:10.1021/pr400714w (2013).
23 Shade, K. T. et al. A single glycan on IgE is indispensable for initiation of anaphylaxis. The Journal of experimental medicine 212, 457-467, doi:10.1084/jem.20142182 (2015).
24 Wu, G. et al. Glycoproteomic studies of IgE from a novel hyper IgE syndrome linked to PGM3 mutation. Glycoconj J 33, 447-456, doi:10.1007/s10719-015-9638-y (2016).
25 Stanley, P., Taniguchi, N. & Aebi, M. in Essentials of Glycobiology (eds rd et al.) 99-111 (2015).
26 Dore, K. A. et al. Thermal sensitivity and flexibility of the Cepsilon3 domains in immunoglobulin E. BiochimBiophys Acta Proteins Proteom 1865, 1336-1347, doi:10.1016/j.bbapap.2017.08.005 (2017).
27 Wuhrer, M. et al. Glycosylation profiling of immunoglobulin G (IgG) subclasses from human serum. Proteomics 7, 4070-4081, doi:10.1002/pmic.200700289 (2007).
28 Nimmerjahn, F. & Ravetch, J. V. Anti-inflammatory actions of intravenous immunoglobulin. Annual review of immunology 26, 513-533 (2008).
29 Colucci, M. et al. Sialylation of N-linked glycans influences the immunomodulatory effects of IgM on T cells. Journal of immunology 194, 151-157, doi:10.4049/jimmunol.1402025 (2015).
30 Ding, J. X. et al. Aberrant sialylation of serum IgA1 was associated with prognosis of patients with IgA nephropathy. Clin Immunol 125, 268-274, doi:10.1016/j.clim.2007.08.009 (2007).
31 Collins, B. E., Smith, B. A., Bengtson, P. & Paulson, J. C. Ablation of CD22 in ligand-deficient mice restores B cell receptor signaling. Nature immunology 7, 199-206, doi:10.1038/ni1283 (2006).
32 Maurer, M. A. et al. Glycosylation of Human IgA Directly Inhibits Influenza A and Other Sialic-Acid-Binding Viruses. Cell Rep 23, 90-99, doi:10.1016/j.celrep.2018.03.027 (2018).
33 Anthony, R. M. et al. Recapitulation of IVIG anti-inflammatory activity with a recombinant IgG Fc. Science 320, 373-376 (2008).
34 Igdoura, S. A. Asialoglycoprotein receptors as important mediators of plasma lipids and atherosclerosis. Curr Opin Lipidol 28, 209-212, doi:10.1097/MOL.0000000000000395 (2017).
35 Fleischer, D. M. et al. Oral food challenges in children with a diagnosis of food allergy. J Pediatr 158, 578-583 e571, doi:10.1016/j.jpeds.2010.09.027 (2011).
36 Hamilton, R. G., MacGlashan, D. W., Jr. & Saini, S. S. IgE antibody-specific activity in human allergic disease. Immunol Res 47, 273-284, doi:10.1007/s12026-009-8160-3 (2010).
37 Christensen, L. H., Holm, J., Lund, G., Riise, E. & Lund, K. Several distinct properties of the IgE repertoire determine effector cell degranulation in response to allergen challenge. The Journal of allergy and clinical immunology 122, 298-304, doi:10.1016/j.jaci.2008.05.026 (2008).
38 Croote, D., Darmanis, S., Nadeau, K. C. & Quake, S. R. High-affinity allergen-specific human antibodies cloned from single IgE B cell transcriptomes. Science 362, 1306-1309, doi:10.1126/science.aau2599 (2018).
39 Henault, J. et al. Self-reactive IgE exacerbates interferon responses associated with autoimmunity. Nature immunology 17, 196-203, doi:10.1038/ni.3326 (2016).
40 Dema, B. et al. Autoreactive IgE is prevalent in systemic lupus erythematosus and is associated with increased disease activity and nephritis. PloS one 9, e90424, doi:10.1371/journal.pone.0090424 (2014).
41 Kojima, S., Yokogawa, M. & Tada, T. Raised levels of serum IgE in human helminthiases Am J Trop Med Hyg 21, 913-918, doi:10.4269/ajtmh.1972.21.913 (1972).
42 Dombrowicz, D. et al. Anaphylaxis mediated through a humanized high affinity IgE receptor. Journal of immunology 157, 1645-1651 (1996).
43 Dombrowicz, D., Flamand, V., Brigman, K. K., Koller, B. H. & Kinet, J. P. Abolition of anaphylaxis by targeted disruption of the high affinity immunoglobulin E receptor alpha chain gene. Cell 75, 969-976 (1993).
44 Kirshenbaum, A. S. et al. Characterization of novel stem cell factor responsive human mast cell lines LAD 1 and 2 established from a patient with mast cell sarcoma/leukemia; activation following aggregation of FcepsilonRl or FcgammaRI. Leukemia research 27, 677-682 (2003).
45 MacGlashan, D., Jr., Lavens-Phillips, S. & Katsushi, M. IgE-mediated desensitization in human basophils and mast cells. Front Biosci 3, d746-756 (1998).
46 Christensen, S. & Egebjerg, J. Cloning, expression and characterization of a sialidase gene from Arthrobacter ureafaciens. Biotechnol Appl Biochem 41, 225-231, doi:10.1042/BA20040144 (2005).

1. A fusion polypeptide comprising:
an Immunolobulin E (IgE) antibody Fc domain region; and
a sialidase or a functional portion thereof, preferably wherein the sialidase or a functional portion thereof can hydrolyze alpha-(2->3)-, alpha-(2->6)-, alpha-(2->8)-glycosidic linkages of terminal sialic residues on IgE.

2. The fusion polypeptide of claim 1, wherein the sialidase is NEU1, NEU2, NEU3, NEU4, or Vibrio cholerae serotype O1 sialidase.

3. The fusion polypeptide of claim 1, wherein the sialidase is a human sialidase.

4. The fusion polypeptide of claim 1, wherein the fusion polypeptide comprises an IgE CH2 region, an IgE CH3 region, and an IgE CH4 region.

5. A polynucleotide encoding the fusion polypeptide of claim 1.

6. A vector comprising a polynucleotide encoding the fusion polypeptide of claim 1.

7. A cell comprising the vector of claim 6, and optionally expressing the fusion polypeptide of claim 1.

8. A method of treating a subject having an IgE-mediated disorder, the method comprising:
administering to the subject an effective amount of a composition comprising the fusion protein of claim 1.

9. The method of claim 8, wherein the IgE-mediated disorder is an allergic disorder.

10. The method of claim 9, wherein the allergic disorder is an anaphylactic allergy.

11. The method of claim 9, wherein the allergic disorder is asthma, atopic dermatitis. allergic rhinitis, allergic conjunctivitis, eczema, or urticaria.

12. A method of preparing glycoengineered IgE, the method comprising:
providing a composition comprising IgE, preferably human IgE, obtained from a plurality of subjects,
contacting the IgE with a sialidase under conditions and for a time sufficient to remove sialylation from the IgE;
thereby preparing glycoengineered IgE.

13. The method of claim 12, wherein the method further comprises formulating the glycoengineered IgE for intravenous administration.

14. A composition comprising the glycoengineered IgE prepared by the method of claim 12, and a pharmaceutically acceptable carrier.

15. A method of treating a subject having an IgE-mediated disorder, the method comprising administering to the subject an effective amount of the composition of claim 12.

16. The method of claim 15, wherein the IgE-mediated disorder is an allergic disorder.

17. The method of claim 16, wherein the allergic disorder is an anaphylactic allergy.

18. The method of claim 16, wherein the allergic disorder is asthma, atopic dermatitis. allergic rhinitis, allergic conjunctivitis, eczema, or urticaria. for use in treating a subject having an IgE-mediated disorder.

19. A composition comprising the fusion polypeptide of claim 1, and a pharmaceutically acceptable carrier.

20.-27. (canceled)


SEQUENCE LISTING (73 sequences)

SEQ ID NO: 1 - protein, 415 aa, Homo sapiens
MTGERPSTALPDRRWGPRILGFWGGCRVWVFAAIFLLLSLAASWSKAENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISVYGTL

SEQ ID NO: 2 - protein, 380 aa, Homo sapiens
MASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 3 - protein, 461 aa, Homo sapiens
MRPADLPPRPMEESPASSSAPTETEEPGSSAEVMEEVTTCSFNSPLFRQEDDRGITYRIPALLYIPPTHTFLAFAEKRSTRRDEDALHLVLRRGLRIGQLVQWGPLKPLMEATLPGHRTMNPCPVWEQKSGCVFLFFICVRGHVTERQQIVSGRNAARLCFIYSQDAGCSWSEVRDLTEEVIGSELKHWATFAVGPGHGIQLQSGRLVIPAYTYYIPSWFFCFQLPCKTRPHSLMIYSDDLGVTWHHGRLIRPMVTVECEVAEVTGRAGHPVLYCSARTPNRCRAEALSTDHGEGFQRLALSRQLCEPPHGCQGSVVSFRPLEIPHRCQDSSSKDAPTIQQSSPGSSLRLEEEAGTPSESWLLYSHPTSRKQRVDLGIYLNQTPLEAACWSRPWILHCGPCGYSDLAALEEEGLFGCLFECGTKQECEQIAFRLFTHREILSHLQGDCTSPGRNPSQFKSN

SEQ ID NO: 4 - protein, 484 aa, Homo sapiens
MGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 5 - protein, 781 aa, Vibrio cholerae
MRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 6 - protein, 324 aa, Homo sapiens
CSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSALPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 7 - protein, 331 aa, Mus sp.
VRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPS

SEQ ID NO: 8 - protein, 327 aa, Canis lupus familiaris
CALNFIPPTVKLFHSSCNPVGDTHTTIQLLCLISGYVPGDMEVIWLVDGQKATNIFPYTAPGTKEGNVTSTHSELNITQGEWVSQKTYTCQVTYQGFTFKDEARKCSESDPRGVTSYLSPPSPLDLYVHKAPKITCLVVDLATMEGMNLTWYRESKEPVNPGPLNKKDHFNGTITVTSTLPVNTNDWIEGETYYCRVTHPHLPKDIVRSIAKAPGKRAPPDVYLFLPPEEEQGTKDRVTLTCLIQNFFPADISVQWLRNDSPIQTDQYTTTGPHKVSGSRPAFFIFSRLEVSRVDWEQKNKFTCQVVHEALSGSRILQKWVSKTPGK

SEQ ID NO: 9 - protein, 327 aa, Felis catus
CTMNFIPPTVKLFHSSCNPLGDTGSTIQLLCLISGYVPGDMEVTWLVDGQKATNIFPYTAPGKQEGKVTSTHSELNITQGEWVSQKTYTCQVTYQGFTFEDHARKCTESDPRGVSTYLSPPSPLDLYVHKSPKITCLVVDLANTDGMILTWSRENGESVHPDPMVKKTQYNGTITVTSTLPVDATDWVEGETYQCKVTHPDLPKDIVRSIAKAPGRRFPPEVYVFLPPEGEPKTKDKVTLTCLIQNFFPPDISVQWLHNDSPVRTEQQATTWPHKATGPSPAFFVFSRLEVSRADWEQRDVFTCQVVHEALPGFRTLKKSVSKNPGK

SEQ ID NO: 10 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 215 aa, Homo sapiens
PPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 278 aa, Homo sapiens
LKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 13 - protein, 210 aa, Homo sapiens
PEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSL

SEQ ID NO: 14 - protein, 227 aa, Mus sp.
VPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 15 - protein, 233 aa, Mus sp.
MPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 16 - protein, 239 aa, Mus sp.
EPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 17 - protein, 233 aa, Mus sp.
EPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 18 - protein, 224 aa, Canis lupus familiaris
PPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 19 - protein, 223 aa, Felis catus
PKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 20 - protein, 732 aa, Artificial Sequence
  Description of Artificial Sequence Synthetic polypeptide
MYRMQLLSCIALSLALVTNSGSPAQWKLENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISVYGTLGGGGGGCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSALPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKHHHHHH

SEQ ID NO: 21 - protein, 724 aa, Artificial Sequence
  Description of Artificial Sequence Synthetic polypeptide
MYRMQLLSCIALSLALVTNSCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSALPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKMASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 22 - protein, 731 aa, Artificial Sequence
  Description of Artificial Sequence Synthetic polypeptide
MPLLLLLPLLWAGALAASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQGGGGGGCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSALPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKHHHHHH

SEQ ID NO: 23 - protein, 813 aa, Artificial Sequence
  Description of Artificial Sequence Synthetic polypeptide
MPLLLLLPLLWAGALAMRPADLPPRPMEESPASSSAPTETEEPGSSAEVMEEVTTCSFNSPLFRQEDDRGITYRIPALLYIPPTHTFLAFAEKRSTRRDEDALHLVLRRGLRIGQLVQWGPLKPLMEATLPGHRTMNPCPVWEQKSGCVFLFFICVRGHVTERQQIVSGRNAARLCFIYSQDAGCSWSEVRDLTEEVIGSELKHWATFAVGPGHGIQLQSGRLVIPAYTYYIPSWFFCFQLPCKTRPHSLMIYSDDLGVTWHHGRLIRPMVTVECEVAEVTGRAGHPVLYCSARTPNRCRAEALSTDHGEGFQRLALSRQLCEPPHGCQGSVVSFRPLEIPHRCQDSSSKDAPTIQQSSPGSSLRLEEEAGTPSESWLLYSHPTSRKQRVDLGIYLNQTPLEAACWSRPWILHCGPCGYSDLAALEEEGLFGCLFECGTKQECEQIAFRLFTHREILSHLQGDCTSPGRNPSQFKSNGGGGGGCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSALPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKHHHHHH

SEQ ID NO: 24 - protein, 836 aa, Artificial Sequence
  Description of Artificial Sequence Synthetic polypeptide
MPLLLLLPLLWAGALAMGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPSGGGGGGCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSALPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKHHHHHH

SEQ ID NO: 25 - protein, 1125 aa, Artificial Sequence
  Description of Artificial Sequence Synthetic polypeptide
MYRMQLLSCIALSLALVTNSCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSALPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKMRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 26 - protein, 739 aa, Artificial Sequence
  Description of Artificial Sequence Synthetic polypeptide
MYRMQLLSCIALSLALVTNSGSPAQWKLENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISVYGTLGGGGGGVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPSHHHHHH

SEQ ID NO: 27 - protein, 740 aa, Artificial Sequence
  Description of Artificial Sequence Synthetic polypeptide
MPLLLLLPLLWAGALADMASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQGGGGGGVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPSHHHHHH

SEQ ID NO: 28 - protein, 820 aa, Artificial Sequence
  Description of Artificial Sequence Synthetic polypeptide
MPLLLLLPLLWAGALAMRPADLPPRPMEESPASSSAPTETEEPGSSAEVMEEVTTCSFNSPLFRQEDDRGITYRIPALLYIPPTHTFLAFAEKRSTRRDEDALHLVLRRGLRIGQLVQWGPLKPLMEATLPGHRTMNPCPVWEQKSGCVFLFFICVRGHVTERQQIVSGRNAARLCFIYSQDAGCSWSEVRDLTEEVIGSELKHWATFAVGPGHGIQLQSGRLVIPAYTYYIPSWFFCFQLPCKTRPHSLMIYSDDLGVTWHHGRLIRPMVTVECEVAEVTGRAGHPVLYCSARTPNRCRAEALSTDHGEGFQRLALSRQLCEPPHGCQGSVVSFRPLEIPHRCQDSSSKDAPTIQQSSPGSSLRLEEEAGTPSESWLLYSHPTSRKQRVDLGIYLNQTPLEAACWSRPWILHCGPCGYSDLAALEEEGLFGCLFECGTKQECEQIAFRLFTHREILSHLQGDCTSPGRNPSQFKSNGGGGGGVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPSHHHHHH

SEQ ID NO: 29 - protein, 843 aa, Artificial Sequence
  Description of Artificial Sequence Synthetic polypeptide
MPLLLLLPLLWAGALAMGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPSGGGGGGVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPSHHHHHH

SEQ ID NO: 30 - protein, 725 aa, Artificial Sequence
  Description of Artificial Sequence Synthetic polypeptide
MYRMQLLSCIALSLALVTNSCALNFIPPTVKLFHSSCNPVGDTHTTIQLLCLISGYVPGDMEVIWLVDGQKATNIFPYTAPGTKEGNVTSTHSELNITQGEWVSQKTYTCQVTYQGFTFKDEARKCSESDPRGVTSYLSPPSPLDLYVHKAPKITCLVVDLATMEGMNLTWYRESKEPVNPGPLNKKDHFNGTITVTSTLPVNTNDWIEGETYYCRVTHPHLPKDIVRSIAKAPGKRAPPDVYLFLPPEEEQGTKDRVTLTCLIQNFFPADISVQWLRNDSPIQTDQYTTTGPHKVSGSRPAFFIFSRLEVSRVDWEQKNKFTCQVVHEALSGSRILQKWVSKTPGKMASCPVLQKEKVFQSGAHAYRIPALLYLPQQKTLLAFAEKRTSKKDEHAELIVLRRGSYDASTHHVQWHAQEVVAQAQLEGHRSMNPSPLYDEKTGTLFLFFIAIPGQVSEHHQLHTRVNVTRLCQVTSTDHGRSWSPARDLTGPAIGAAHKEWATFAVGPGHCLQLHNKAQSLVVPAYAYRNLHSLPRPSPFAFCFVSHDHGHTWERGNFVAQDTLECQVAEVGGAERRVVYLNARSPRRARVQAESTNEGLDFTEAQVVKELVEPPHGCQGSVISFPNPHAGSGACDRWLLYTHPTDPQRRAHLGAYLHRRRPGREAWSEPVPLAAGGCAYSDLQAMGAGPDGSPQFGCLYESGHYEEVVFLMFTLKQAFPAEFVS

SEQ ID NO: 31 - protein, 726 aa, Artificial Sequence
  Description of Artificial Sequence Synthetic polypeptide
MYRMQLLSCIALSLALVTNSCTMNFIPPTVKLFHSSCNPLGDTGSTIQLLCLISGYVPGDMEVTWLVDGQKATNIFPYTAPGKQEGKVTSTHSELNITQGEWVSQKTYTCQVTYQGFTFEDHARKCTESDPRGVSTYLSPPSPLDLYVHKSPKITCLVVDLANTDGMILTWSRENGESVHPDPMVKKTQYNGTITVTSTLPVDATDWVEGETYQCKVTHPDLPKDIVRSIAKAPGRRFPPEVYVFLPPEGEPKTKDKVTLTCLIQNFFPPDISVQWLHNDSPVRTEQQATTWPHKATGPSPAFFVFSRLEVSRADWEQRDVFTCQVVHEALPGFRTLKKSVSKNPGKMESCPILQKERVFQSGAHAYRIPALLYLPEQKTVLAFAEQRISKKDEHAELIVLRRGSYDASTHQVQWHAQEVVAQAQLEGHRSMNPSPLYDEKTGTLFLFFIAIPGQVSEHHQLHTRVNVTRLCQVTSPDHGRSWSPARDLTDPAIGTAHKEWATFAVGPGHCLQLGNKTRSLLVPAYAYRNLHPHRRPSPFAFCFVSHDHGHTWQRGNFVAQDTVECQVAEVGGAEQRVVYLNARSPLRARVQAESTNEGLDFKEAQLVEELVEPPHGCQGSIVSFPGPGAGADASDRWLLYTHPTDPRQRAHLGAYLSKPRPGPAAWSEPALLATGSCAYSDLQSMGTGPDGSPQFGCLYESDDYEAVMFLTFTLKQAFPAEFLSL

SEQ ID NO: 32 - protein, 20 aa, Artificial Sequence
  Description of Artificial Sequence Synthetic peptide
MYRMQLLSCIALSLALVTNS

SEQ ID NO: 33 - protein, 16 aa, Artificial Sequence
  Description of Artificial Sequence Synthetic peptide
MPLLLLLPLLWAGALA

SEQ ID NO: 34 - protein, 39 aa, Artificial Sequence
  Description of Artificial Sequence Synthetic polypeptide
METDTLLLWVLLLWVPGSTGDAAQPARRAVRSLVPSSDP

SEQ ID NO: 35 - protein, 5 aa, Artificial Sequence
  Description of Artificial Sequence Synthetic peptide
GGGGS

SEQ ID NO: 36 - protein, 4 aa, Artificial Sequence
  Description of Artificial Sequence Synthetic peptide
GGGS

SEQ ID NO: 37 - protein, 6 aa, Artificial Sequence
  Description of Artificial Sequence Synthetic peptide
GGGGGG

SEQ ID NO: 38 - protein, 5 aa, Artificial Sequence
  Description of Artificial Sequence Synthetic peptide
GSGGS

SEQ ID NO: 39 - protein, 4 aa, Artificial Sequence
  Description of Artificial Sequence Synthetic peptide
GGSG

SEQ ID NO: 40 - protein, 5 aa, Artificial Sequence
  Description of Artificial Sequence Synthetic peptide
GGSGG

SEQ ID NO: 41 - protein, 5 aa, Artificial Sequence
  Description of Artificial Sequence Synthetic peptide
GSGSG

SEQ ID NO: 42 - protein, 5 aa, Artificial Sequence
  Description of Artificial Sequence Synthetic peptide
GSGGG

SEQ ID NO: 43 - protein, 5 aa, Artificial Sequence
  Description of Artificial Sequence Synthetic peptide
GGGSG

SEQ ID NO: 44 - protein, 5 aa, Artificial Sequence
  Description of Artificial Sequence Synthetic peptide
GSSSG

SEQ ID NO: 45 - protein, 415 aa, Canis lupus familiaris
MAGRPGAALPGRPGGPRVPGCWGGWRVRARAATFLLLWPLAGLGAGIKNDFSLVQPLVTMEQLLWVSGRQIGNVHTFRIPLITATPRGTLLAFAEARKMSTSDEGAKFIALRRSTDEGSTWSPTAFIVNDGETPDGLNLGAVVSDMETGVVFLFYSLCAHKAGCQVASTMLVWSKDDGVSWSPPRNLSLDIGTEMFAPGPGSGIQKRHDPRKGRLIVCGHGTLERDGVFCLLSDDHGASWRYGSGVSGIPYGQPKRENDFNPDECQPYELPDGSVVINARNQNNYHCHCRIVLRSYDACDTIRPRDVTFDPELVDPVVAAGAVATGSGLVFFSNPAHPEFRVNLTLRWSFSNGTSWRKETVQLWPGPSGYSSLAALEGSVGGKDQAPQLYVLYEKGRNQYTESISLAKVSVYGTL

SEQ ID NO: 46 - protein, 378 aa, Canis lupus familiaris
MASCPVLQKEKVFQSGAHAYRIPALLYLPQQKTLLAFAEKRTSKKDEHAELIVLRRGSYDASTHHVQWHAQEVVAQAQLEGHRSMNPSPLYDEKTGTLFLFFIAIPGQVSEHHQLHTRVNVTRLCQVTSTDHGRSWSPARDLTGPAIGAAHKEWATFAVGPGHCLQLHNKAQSLVVPAYAYRNLHSLPRPSPFAFCFVSHDHGHTWERGNFVAQDTLECQVAEVGGAERRVVYLNARSPRRARVQAESTNEGLDFTEAQVVKELVEPPHGCQGSVISFPNPHAGSGACDRWLLYTHPTDPQRRAHLGAYLHRRRPGREAWSEPVPLAAGGCAYSDLQAMGAGPDGSPQFGCLYESGHYEEVVFLMFTLKQAFPAEFVS

SEQ ID NO: 47 - protein, 438 aa, Canis lupus familiaris
MEEPESRGEVMEEVTSCSFNSPLFQQEDKRGITYRIPALLYIPPTHTFLAFAEKRSTSRDEDALHLVLRRGLRTGHSVQWEPLKPLMEAMLPGHRTMNPCPVWEQKSGCVYLFFICVRGHVTERQQVMSGRNAARLCFICSQDAGCSWSEVRDLTEEVIGAEVKRWATFAVGPGHGIQLQSGRLIIPAYAYYIPYWFFCFRLPCKAKPHSLMIYSDDLGATWHHGRLIKPTATVECEVAEVTGRAGDPVLYCSARTPNRCRAEAFSINNGECFQRPALSNQLCEPPHGCQGSVVSFRPLEILHGYQDLNGKDSPTIQQSPLLDSSLWPEQEGGILSKSWLLYSHPTSKKRRVNLGIYLNQTPLDAACWSRPWILHCGPCGYSDLAALEKEGLFGCLFECGTKRECEQIAFRLFTDREILSHMHEDSTGPGKNSEPTES

SEQ ID NO: 48 - protein, 417 aa, Felis catus
MMADRAGQVLLGRPGDPRMPGFWGGCRVQVLATIIFLLLLSLAESGARAKNDFSLVQPLVTMEQLLWVSGRQIGSVHTFRIPLITTTPQGTLLAFAEARKMSTSDEGAKFIALRRSLDQGSTWSPTAFIVDDGDTPDGLNLGAVVNDMETGVVFLFYSLCAHKAGCQVASTMLVWSKDDGVSWSPPRNLSLDIGTEMFAPGPGSGIQKQREPRKGRLIVCGHGTLERDGVFCLLSDDHGATWRYGSGVSGIPYGQPKRENDFNPDECQPYELPDGSVVINARNQNNYHCHCRIVLRSYDACDTIRPRDVTFDPELVDPVVAAGAVATSSGIVFFSNPAHPEFRVNLTLRWSFSNGTSWRKETVQLWPGPSGYSSLAALEGNVGGKDQAPQLYVLYEKGRNQYTESISLAKVSVYGTL

SEQ ID NO: 49 - protein, 379 aa, Felis catus
MESCPILQKERVFQSGAHAYRIPALLYLPEQKTVLAFAEQRISKKDEHAELIVLRRGSYDASTHQVQWHAQEVVAQAQLEGHRSMNPSPLYDEKTGTLFLFFIAIPGQVSEHHQLHTRVNVTRLCQVTSPDHGRSWSPARDLTDPAIGTAHKEWATFAVGPGHCLQLGNKTRSLLVPAYAYRNLHPHRRPSPFAFCFVSHDHGHTWQRGNFVAQDTVECQVAEVGGAEQRVVYLNARSPLRARVQAESTNEGLDFKEAQLVEELVEPPHGCQGSIVSFPGPGAGADASDRWLLYTHPTDPRQRAHLGAYLSKPRPGPAAWSEPALLATGSCAYSDLQSMGTGPDGSPQFGCLYESDDYEAVMFLTFTLKQAFPAEFLSL

SEQ ID NO: 50 - protein, 438 aa, Felis catus
MEELGSREEVMEEVTSCSFSSPLFQQEDKRGITYRIPALLYIPPTHTFLAFAEKRSTSRDEDALHLVLRRGLRTGHSVQWGPLKPLMEATLPGHRTMNPCPVWEQKSGCVYLFFICVRGHVTERQQIMSGRNAARLCFICSRDTGCSWSEVRDLTEEVIGAEVKRWATFAVGPGHGIQLQSGRLVIPAYAYYIPYWFFCFRLPCKAKPHSLMIYSDDLGATWHHGRLIQPMATVECEVAEVTGRGGNPVLYCSARTPNRCRAEAFSDDRGECFQRLALSRELCEPPHGCQGSVVSFRPPEILRGCQDPNGKDASTIQQSPLLDRSLRLEQEAGTLSESWLLYSHPTCKKRRVNLGIYLNQTPLDAACWSHPWILHCGPCGYSDLAALEKEGLFGCLFECGTKRECEQIAFRLFTDREILSHVHGDFTSPDKNPEPIES

SEQ ID NO: 51 - protein, 346 aa, Felis catus
  MOD_RES (3)..(3): Any amino acid
TWXAARDLTEEAVGGAEQDWATFAVGPGHGIQLRSGRLLVPAYTYHVDRRECFGKICRTSPHAFTFYSDDHGRTWRHGGLVPNLRSGECQLAAVDRGQAGHILYCNARSPLGSRVQALSADEGTSFLPGELVPALAETARGCQGSVLGFPAPLFSRPRNGRWPVGAGSHSYSPHLCLGVQEPSEEGTGDTQGAQGPSGMEGCRDSPEEPGLGPGLSGIMAAWTSKVPGPPATLSQSPTWLLYSHPVGRRARLHMGIYLSRSPLDPHSWTEPWVIYEGPSGYSDLASVGPTVKGALTFACLFESGARVSYEEISFCMFSLGEVLQNLPPGPKPPGLGDKPLERCRPS

SEQ ID NO: 52 - protein, 425 aa, Bos taurus
SIQAPSIYPLRLCCTEEARVRLGCLVKDYLPGSVTVTWDTVPLDGSTLTFPSIQMASSSLYITTSQLTISGEQSKEFTCRVLHPETNSNLNKTISTECAKNFSDPSVKLFYSSCNPNGDTQTTIHLLCRISGYTPGKIKVTWLVDGLQSEELYARSGPEIQEGNLTSTYSEVNITQGQWVSEKTYTCRVNYYGYNFESHAHRCTAESEPRGVSAYLSPPTPLELYVNKSPKITCLVVDLANEKNLSLTWSRANGKPVHAGPPEIKRQFNGTVTVTSTLPVDVTDWVEGETYYCKVSHSDLPTDIQRSISKDVGKRLAPKAYVFLPDGKELENEEELTLTCMIQKFFPRDIFVRWLHNKELMRADQHTTTQPHRADNNTPAFFAYSRLAVPRANWKRGDEFTCQVIHEALPRTRTLEKSVFINSGK

SEQ ID NO: 53 - protein, 428 aa, Bos taurus
MEEVTSCSFSSPLFQQEDKRGVTYRIPALIYVPPAHTFLAFAEKRSSSKDEDALHLVLRRGLRTGQSVQWEPLKSLMKATLPGHRTMNPCPVWERKSGYVYLFFICVQGHVTERQQIMSGRNPARLCFICSQDAGYSWSDVRDLTEEVIGPEVTHWATFAVGPGHGIQLQSGRLIIPAYAYYIPFWFFCFRLPYRARPHSLMIYSDDLGATWHHGRLIKPMVTVECEVAEVIGKAGHPVLYCSARTPNRHRAEALSIDHGECFQKPVLSHQLCEPPHGCQGSVVSFCPLEIPGGCQDLAGEDAPAIQQSPLLCSSVRPEPEAGTLSESWLLYSHPTNKKRRVDLGIYLNQSPLEAACWSRPWILHCGPCGYSDLAALENEGLFGCLFECGTKQECEQIAFRLFTDREILSHVQGDCSTPGMNSEPSKK

SEQ ID NO: 54 - protein, 415 aa, Bos taurus
MTEEGPGIVSLGKLRRPRMLRLWGICRVQIFSAIFMLMSPAGVGAGAKDDFSLVHPLVTMEQLLWVSGKQIGSVDTFRIPLITTTPRGTLLAFAEARKMSTSDKGAKFIALRRSMDQGSTWSPTAFIVDDGETPDGLNLGAVVSDTTTGVVFLFYSLCAHKAGCQVASTMLVWSKDDGVSWSSPRNLSLDIGTEMFAPGPGSGIQKQREPRKGRLIVCGHGTLERDGVFCLLSDDHGVSWRYGGGVSGIPYGQPKRENDFNPDECQPYELPDGSVVINARNQNNYHCHCRIILRSYDACDTLRPRDVTFDTELVDPVVAAGAVATSSGIVFFSNPAHPEFRVNLTLRWSFSNGTSWRKETVQLWPGPSGYSSLTTLEGNVDGKDEAPQLYVLYEKGRNQYMESISLVKVSVYGTL

SEQ ID NO: 55 - protein, 424 aa, Equus caballus
VSKQAPLILPLAACCKDTKTTNITLGCLVKGYFPEPVTVTWDAGSLNRSTMTFPAVFDQTSGLYTTISRVVASGKWAKQKFTCNVVHSQETFNKTFNACIVTFTPPTVKLFHSSCDPGGDSHTTIQLLCLISDYTPGDIDIVWLIDGQKVDEQFPQHGLVKQEGKLASTHSELNITQGQWASENTYTCQVTYKDMIFKDQARKCTESDPRGVSVYLSPPSPLDLYVSKSPKITCLVVDLANVQGLSLNWSRESGEPLQKHTLATSEQFNKTFSVTSTLPVDTTDWIEGETYKCTVSHPDLPREVVRSIAKAPGKRLSPEVYVFLPPEEDQSSKDKVTLTCLIQNFFPADISVQWLRNNVLIQTDQQATTRPQKANGPNPAFFVFSRLEVSRAEWEQKNKFACKVVHEALSQRTLQKEVSKDPGK

SEQ ID NO: 56 - protein, 416 aa, Equus caballus
MMAERSGTVLRDRPGGPRTLGFWGGCRVQVLAAIFLLLLSLARFGTRAGNDFDLVQPLVTMEQLLWVSGKQIGSVDTFRIPLITVTPQGTLLAFAEARKMSSSDLGAKFIALRRSMDQGSTWSPTAFIVDDGETPDGLNLGAVVSDRETGIVFLFYSLCAHKAGCQVASTMLVWSKDDGVSWSSPRNLSLDIGTEVFAPGPGSGIQKQRQPRKGRLIVCGHGTLERDGVFCLLSDDHGASWRYGNGIRGIPYGQPKQKYDFNPDECQPYELPDGSVVINARNQNNYHCHCRIILRSYDACDTLRPRDVTFDPELVDPVVAAGAVATNSGIVFFSNPAHPEFRVNLTLRWSFSNGTSWRKETVQLWPGPSGYSSMAALEGSLGGEDQAPQLYVLYEKGLSQYTESISLAKVSVYGTL

SEQ ID NO: 57 - protein, 379 aa, Equus caballus
MASCPILQKEKVFQSGAHAYRIPALLYLPQQKTLLAFAEKRRDKRDEHAELIALRKGSYDVATHRVLWHAEEVVTQAQLEGHRSMNPSPLYDKKTGTLFLFFISIPGQVSERHQLHTRVNVTRLCQVTSTDHGRSWSPARDVTDSIIGSAYKEWATFALGPGHCLQLHNESQSLVVPAYAYRNLHPRHRPAPFAVCFISHDHGHTWQRGNFVAQDTLECQVAEVGSTKQRVVYLNARSPLRARVQAESNNDGLDFKEIQLVTKLAEPPQGCQGSIVSFPNPTVQSDFSDKWLLYTHPTDSRQRSNLGVYLNKWPLAPTAWSEPTLLAKGSCAYSDLQSMGAGPDGSPQFACLYESNNYEEIIFLMFTLKQAFPAEFLPQ

SEQ ID NO: 58 - protein, 428 aa, Equus caballus
MEEVTSCSFNSPLFQQEDKRGVTYRIPALLYVPPTHTFLAFAEKRSTCRDEDALHLVLRRGVRTEHSVQWGPLKPLMEATLPGHRTMNPCPVWEQKSGCVYLFFICVRDHVTERQQIVSGRNAARLCFICSQDAGCSWSELRDLTEEVIGPEMKRWATFAVGPGHGIQLQSGRLVIPAYAYYIPYLFFCFRLPCKARPHSLMIYSDDLGATWHHGRLIKPMVTVECEVAEVTERTGYPVLYCSARTPNRCRAEALSTDHGECFQRPALSQQLCEPPHGCQGSVVSFQPLEIPHGYQDPSGQDAPTIQQNPLLDSSLRLEQEAGTLSQSWLLYSHPTSKKRRVNLGIYLNQTPLEATCWSRPWILHCGPCGYSDLAALEKEGLFACLFECGIKRECEQIAFRLFTVQEIVSHVQGDCTSPGRISEPIQN

SEQ ID NO: 59 - DNA, 2199 bp, Artificial Sequence
  Description of Artificial Sequence Synthetic polynucleotide
atgtacaggatgcaactcctgtcttgcattgcactaagtcttgcacttgtcacgaattcgggatccccagcacagtggaagcttgagaacgacttcggtctggtgcagccgctggtgaccatggagcaactgctgtgggtgagcgggagacagatcggctcagtggacaccttccgcatcccgctcatcacagccactccgcggggcactcttctcgcctttgctgaggcgaggaaaatgtcctcatccgatgagggggccaagttcatcgccctgcggaggtccatggaccagggcagcacatggtctcctacagcgttcattgtcaatgatggggatgtccccgatgggctgaaccttggggcagtagtgagcgatgttgagacaggagtagtatttcttttctactccctttgtgctcacaaggccggctgccaggtggcctctaccatgttggtatggagcaaggatgatggtgtttcctggagcacaccccggaatctctccctggatattggcactgaagtgtttgcccctggaccgggctctggtattcagaaacagcgggagccacggaagggccgcctcatcgtgtgtggccatgggacgctggagcgggacggagtcttctgtctcctcagcgatgatcatggtgcctcctggcgctacggaagtggggtcagcggcatcccctacggtcagcccaagcaggaaaatgatttcaatcctgatgaatgccagccctatgagctcccagatggctcagtcgtcatcaatgcccgaaaccagaacaactaccactgccactgccgaattgtcctccgcagctatgatgcctgtgatacactaaggccccgtgatgtgaccttcgaccctgagctcgtggaccctgtggtagctgcaggagctgtagtcaccagctccggcattgtcttcttctccaacccagcacatccagagttccgagtgaacctgaccctgcgatggagcttcagcaatggtacctcatggcggaaagagacagtccagctatggccaggccccagtggctattcatccctggcaaccctggagggcagcatggatggagaggagcaggccccccagctctacgtcctgtatgagaaaggccggaaccactacacagagagcatctccgtggccaaaatcagtgtctatgggacactcggaggaggaggaggaggatgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccgccctgccggtgggcacccgagactggatcgagggggagacctaccagtgcagggtgacccacccccacctgcccagggccctcatgcggtccacgaccaagaccagcggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaacaccatcaccatcaccattga

SEQ ID NO: 60 - DNA, 2196 bp, Artificial Sequence
  Description of Artificial Sequence Synthetic polynucleotide
atgcctctgctgctgctgctgccactgctgtgggctggcgctctggctgcttctctgccagtgctgcagaaggagtccgtgtttcagagcggagctcacgcttacaggatccccgccctgctgtatctgcctggacagcagtccctgctggccttcgctgagcagagggccagcaagaaggacgagcacgctgagctgatcgtgctgaggaggggcgactacgatgctcctacccatcaggtgcagtggcaggctcaggaggtggtggctcaggctaggctggacggacaccggagcatgaacccatgccctctgtatgatgcccagaccggcacactgtttctgttctttatcgctatcccaggccaggtgaccgagcagcagcagctgcagacaagagccaatgtgacccgcctgtgccaggtgacctctacagaccatggaagaacctggtccagcccaagggacctgacagatgctgctatcggccctgcctacagagagtggagcacctttgctgtgggaccaggacactgcctgcagctgcatgatagagcccgctctctggtggtgccagcctacgcttatagaaagctgcacccaatccagaggccaatcccatccgccttctgctttctgtctcacgaccatggaaggacctgggctaggggacatttcgtggctcaggatacactggagtgtcaggtggctgaggtggagaccggagagcagagggtggtgacactgaacgctcggtctcacctgagggctagggtgcaggctcagtccaccaatgacggcctggattttcaggagagccagctggtgaagaagctggtggagccacctccacagggatgtcagggatccgtgatcagcttcccttctccacgctccggaccaggatctccagctcagtggctgctgtacacacacccaacccattcctggcagagagccgacctgggagcttatctgaacccccgcccccctgctcctgaggcttggtccgagcccgtgctgctggctaagggatcttgcgcttactccgacctgcagagcatgggaaccggaccagatggaagcccactgtttggctgtctgtacgaggctaatgattatgaggagatcgtgttcctgatgtttacactgaagcaggccttcccagctgagtatctgccacagggaggaggaggaggaggatgctctagggactttacaccacccaccgtgaagatcctgcagtcttcctgcgatggcggcggccacttccctccaaccatccagctgctgtgcctggtgtccggctacacacccggcaccatcaacatcacctggctggaggacggccaggtcatggacgtggatctgagcacagcctctaccacacaggagggcgagctggcttccacacagagcgagctgaccctgtcccagaagcactggctgagcgatcggacctacacatgccaggtgacatatcagggccataccttcgaggacagcacaaagaagtgtgccgattctaaccctaggggcgtgtccgcttatctgtctcggccctcccctttcgacctgtttatcagaaagagcccaaccatcacatgtctggtggtggatctggccccctctaagggcacagtgaatctgacctggagccgcgcttctggcaagcccgtgaaccattccaccaggaaggaggagaagcagcggaatggcaccctgacagtgaccagcgccctgcctgtgggcacaagggactggatcgagggcgagacctaccagtgccgggtgacacaccctcatctgccaagagctctgatgcgctctaccacaaagacctccggccctagagccgctccagaggtgtatgcctttgctacccccgagtggcctggctccagagataagcgcacactggcctgtctgatccagaacttcatgcctgaggacatctctgtgcagtggctgcacaatgaggtgcagctgcctgatgctagacattccaccacacagccacgcaagaccaagggcagcggcttcttcgtgttctccaggctggaggtgacaagggctgagtgggagcagaaggacgagtttatctgcagagctgtgcacgaggccgctagcccttctcagaccgtgcagcgcgccgtgagcgtgaatccaggcaagcaccatcaccatcaccattga

SEQ ID NO: 61 - DNA, 2442 bp, Artificial Sequence
  Description of Artificial Sequence Synthetic polynucleotide
atgcctctgctgctgctgctgccactgctgtgggctggcgctctggctatgagacctgcggacctgcccccgcgccccatggaagaatccccggcgtccagctctgccccgacagagacggaggagccggggtccagtgcagaggtcatggaagaagtgacaacatgctccttcaacagccctctgttccggcaggaagatgacagagggattacctaccggatcccagccctgctctacataccccccacccacaccttcctggcctttgcagagaagcgttctacgaggagagatgaggatgctctccacctggtgctgaggcgagggttgaggattgggcagttggtacagtgggggcccctgaagccactgatggaagccacactaccggggcatcggaccatgaacccctgtcctgtatgggagcagaagagtggttgtgtgttcctgttcttcatctgtgtgcggggccatgtcacagagcgtcaacagattgtgtcaggcaggaatgctgcccgcctttgcttcatctacagtcaggatgctggatgttcatggagtgaggtgagggacttgactgaggaggtcattggctcagagctgaagcactgggccacatttgctgtgggcccaggtcatggcatccagctgcagtcagggagactggtcatccctgcgtatacctactacatcccttcctggttcttttgcttccagctaccatgtaaaaccaggcctcattctctgatgatctacagtgatgacctaggggtcacatggcaccatggtagactcattaggcccatggttacagtagaatgtgaagtggcagaggtgactgggagggctggccaccctgtgctatattgcagtgcccggacaccaaacaggtgccgggcagaggcgctcagcactgaccatggtgaaggctttcagagactggccctgagtcgacagctctgtgagcccccacatggttgccaagggagtgtggtaagtttccggcccctggagatcccacataggtgccaggactctagcagcaaagatgcacccaccattcagcagagctctccaggcagttcactgaggctggaggaggaagctggaacaccgtcagaatcatggctcttgtactcacacccaaccagtaggaaacagagggttgacctaggtatctatctcaaccagacccccttggaggctgcctgctggtcccgcccctggatcttgcactgtgggccctgtggctactctgatctggctgctctggaggaggagggcttgtttgggtgtttgtttgaatgtgggaccaagcaagagtgtgagcagattgccttccgcctgtttacacaccgggagatcctgagtcacctgcagggggactgcaccagccctggtaggaacccaagccaattcaaaagcaatggaggaggaggaggaggatgctctagggactttacaccacccaccgtgaagatcctgcagtcttcctgcgatggcggcggccacttccctccaaccatccagctgctgtgcctggtgtccggctacacacccggcaccatcaacatcacctggctggaggacggccaggtcatggacgtggatctgagcacagcctctaccacacaggagggcgagctggcttccacacagagcgagctgaccctgtcccagaagcactggctgagcgatcggacctacacatgccaggtgacatatcagggccataccttcgaggacagcacaaagaagtgtgccgattctaaccctaggggcgtgtccgcttatctgtctcggccctcccctttcgacctgtttatcagaaagagcccaaccatcacatgtctggtggtggatctggccccctctaagggcacagtgaatctgacctggagccgcgcttctggcaagcccgtgaaccattccaccaggaaggaggagaagcagcggaatggcaccctgacagtgaccagcgccctgcctgtgggcacaagggactggatcgagggcgagacctaccagtgccgggtgacacaccctcatctgccaagagctctgatgcgctctaccacaaagacctccggccctagagccgctccagaggtgtatgcctttgctacccccgagtggcctggctccagagataagcgcacactggcctgtctgatccagaacttcatgcctgaggacatctctgtgcagtggctgcacaatgaggtgcagctgcctgatgctagacattccaccacacagccacgcaagaccaagggcagcggcttcttcgtgttctccaggctggaggtgacaagggctgagtgggagcagaaggacgagtttatctgcagagctgtgcacgaggccgctagcccttctcagaccgtgcagcgcgccgtgagcgtgaatccaggcaagcaccatcaccatcaccattga

SEQ ID NO: 62 - DNA, 2511 bp, Artificial Sequence
  Description of Artificial Sequence Synthetic polynucleotide
atgcctctgctgctgctgctgccactgctgtgggctggcgctctggctatgggggtccctcgtaccccttcacggacagtgctcttcgagcgggagaggacgggcctgacctaccgcgtgccctcgctgctccccgtgccccccgggcccaccctgctggcctttgtggagcagcggctcagccctgacgactcccacgcccaccgcctggtgctgaggaggggcacgctggccgggggctccgtgcggtggggtgccctgcacgtgctggggacagcagccctggcggagcaccggtccatgaacccctgccctgtgcacgatgctggcacgggcaccgtcttcctcttcttcatcgcggtgctgggccacacgcctgaggccgtgcagatcgccacgggaaggaacgccgcgcgcctctgctgtgtggccagccgtgacgccggcctctcgtggggcagcgcccgggacctcaccgaggaggccatcggtggtgccgtgcaggactgggccacattcgctgtgggtcccggccacggtgtgcagctgccctcaggccgcctgctggtacccgcctacacctaccgcgtggaccgccgagagtgttttggcaagatctgccggaccagccctcactccttcgccttctacagcgatgaccacggccgcacctggcgctgtggaggcctcgtgcccaacctgcgctcaggcgagtgccagctggcagcggtggacggtgggcaggccggcagcttcctctactgcaatgcccggagcccactgggcagccgtgtgcaggcgctcagcactgacgagggcacctccttcctgcccgcagagcgcgtggcttccctgcccgagactgcctggggctgccagggcagcatcgtgggcttcccagcccccgcccccaacaggccacgggatgacagttggtcagtgggccccgggagtcccctccagcctccactcctcggtcctggagtccacgaacccccagaggaggctgctgtagacccccgtggaggccaggtgcctggtgggcccttcagccgtctgcagcctcggggggatggccccaggcagcctggccccaggcctggggtcagtggggatgtggggtcctggaccctggcactccccatgccctttgctgccccgccccagagccccacgtggctgctgtactcccacccagtggggcgcagggctcggctacacatgggtatccgcctgagccagtccccgctggacccgcgcagctggacagagccctgggtgatctacgagggccccagcggctactccgacctggcgtccatcgggccggcccctgaggggggcctggtttttgcctgcctgtacgagagcggggccaggacctcctatgatgagatttccttttgtacattctccctgcgtgaggtcctggagaacgtgcccgccagccccaaaccgcccaaccttggggacaagcctcgggggtgctgctggccctccggaggaggaggaggaggatgctctagggactttacaccacccaccgtgaagatcctgcagtcttcctgcgatggcggcggccacttccctccaaccatccagctgctgtgcctggtgtccggctacacacccggcaccatcaacatcacctggctggaggacggccaggtcatggacgtggatctgagcacagcctctaccacacaggagggcgagctggcttccacacagagcgagctgaccctgtcccagaagcactggctgagcgatcggacctacacatgccaggtgacatatcagggccataccttcgaggacagcacaaagaagtgtgccgattctaaccctaggggcgtgtccgcttatctgtctcggccctcccctttcgacctgtttatcagaaagagcccaaccatcacatgtctggtggtggatctggccccctctaagggcacagtgaatctgacctggagccgcgcttctggcaagcccgtgaaccattccaccaggaaggaggagaagcagcggaatggcaccctgacagtgaccagcgccctgcctgtgggcacaagggactggatcgagggcgagacctaccagtgccgggtgacacaccctcatctgccaagagctctgatgcgctctaccacaaagacctccggccctagagccgctccagaggtgtatgcctttgctacccccgagtggcctggctccagagataagcgcacactggcctgtctgatccagaacttcatgcctgaggacatctctgtgcagtggctgcacaatgaggtgcagctgcctgatgctagacattccaccacacagccacgcaagaccaagggcagcggcttcttcgtgttctccaggctggaggtgacaagggctgagtgggagcagaaggacgagtttatctgcagagctgtgcacgaggccgctagcccttctcagaccgtgcagcgcgccgtgagcgtgaatccaggcaagcaccatcaccatcaccattga

SEQ ID NO: 63 - DNA, 2220 bp, Artificial Sequence
  Description of Artificial Sequence Synthetic polynucleotide
atgtacaggatgcaactcctgtcttgcattgcactaagtcttgcacttgtcacgaattcgggatccccagcacagtggaagcttgagaacgacttcggtctggtgcagccgctggtgaccatggagcaactgctgtgggtgagcgggagacagatcggctcagtggacaccttccgcatcccgctcatcacagccactccgcggggcactcttctcgcctttgctgaggcgaggaaaatgtcctcatccgatgagggggccaagttcatcgccctgcggaggtccatggaccagggcagcacatggtctcctacagcgttcattgtcaatgatggggatgtccccgatgggctgaaccttggggcagtagtgagcgatgttgagacaggagtagtatttcttttctactccctttgtgctcacaaggccggctgccaggtggcctctaccatgttggtatggagcaaggatgatggtgtttcctggagcacaccccggaatctctccctggatattggcactgaagtgtttgcccctggaccgggctctggtattcagaaacagcgggagccacggaagggccgcctcatcgtgtgtggccatgggacgctggagcgggacggagtcttctgtctcctcagcgatgatcatggtgcctcctggcgctacggaagtggggtcagcggcatcccctacggtcagcccaagcaggaaaatgatttcaatcctgatgaatgccagccctatgagctcccagatggctcagtcgtcatcaatgcccgaaaccagaacaactaccactgccactgccgaattgtcctccgcagctatgatgcctgtgatacactaaggccccgtgatgtgaccttcgaccctgagctcgtggaccctgtggtagctgcaggagctgtagtcaccagctccggcattgtcttcttctccaacccagcacatccagagttccgagtgaacctgaccctgcgatggagcttcagcaatggtacctcatggcggaaagagacagtccagctatggccaggccccagtggctattcatccctggcaaccctggagggcagcatggatggagaggagcaggccccccagctctacgtcctgtatgagaaaggccggaaccactacacagagagcatctccgtggccaaaatcagtgtctatgggacactcggaggaggaggaggaggcgtgagaccagtgaacatcacagagcccaccctggagctgctgcactcttcctgcgatcccaatgccttccatagcacaatccagctgtactgttttatctatggccacatcctgaacgacgtgagcgtgtcttggctgatggacgatcgcgagatcacagataccctggctcagaccgtgctgatcaaggaggagggcaagctggccagcacatgctctaagctgaatatcaccgagcagcagtggatgtccgagtccaccttcacctgcaaggtgacatctcagggcgtggactacctggctcacaccagacgctgtcctgatcatgagccaaggggcgtgatcacatacctgatcccaccctctccactggacctgtatcagaacggcgcccccaagctgacctgtctggtggtggatctggagtccgagaagaacgtgaatgtgacatggaatcaggagaagaagacctccgtgtccgccagccagtggtacacaaagcaccataacaatgccaccacatccatcaccagcatcctgccagtggtggctaaggactggatcgagggctacggctatcagtgcatcgtggaccatcccgatttccctaagccaatcgtgaggagcatcacaaagacccctggacagcggtctgccccagaggtgtacgtgttccctccacccgaggaggagtctgaggataagcggacactgacctgtctgatccagaacttctttcctgaggacatcagcgtgcagtggctgggcgatggcaagctgatctctaattcccagcactctaccacaaccccactgaagtctaacggctccaatcagggcttctttatcttctccaggctggaggtggctaagacactgtggacccagcggaagcagtttacatgccaggtcatccatgaggccctgcagaagcccagaaagctggagaagacaatcagcacctctctgggcaacacctccctgcgccctagccaccatcaccatcaccattga

SEQ ID NO: 64 - DNA, 2223 bp, Artificial Sequence
  Description of Artificial Sequence Synthetic polynucleotide
atgcctctgctgctgctgctgccactgctgtgggctggcgctctggctgacatggctagcctgcctgtgctgcagaaggagagcgtgttccagtctggagctcacgcttacaggatccccgccctgctgtatctgcctggccagcagtccctgctggcttttgccgagcagcgggctagcaagaaggatgagcacgctgagctgatcgtgctgaggaggggcgactacgatgctccaacccatcaggtgcagtggcaggctcaggaggtggtggctcaggctagactggacggacaccgctccatgaacccttgcccactgtatgatgctcagacaggcaccctgttcctgttctttatcgctatcccaggacaggtgaccgagcagcagcagctgcagacaagggccaatgtgacccggctgtgccaggtgacaagcaccgaccatggcagaacatggtccagccctagggacctgaccgatgctgctatcggcccagcttacagagagtggtccaccttcgccgtgggaccaggacactgcctgcagctgcatgatagggctcggagcctggtggtgccagcttacgcctataggaagctgcaccctatccagcggcccatcccttctgccttctgctttctgtcccacgaccatggcaggacatgggctcggggccattttgtggcccaggataccctggagtgtcaggtggctgaggtggagacaggcgagcagagagtggtgaccctgaacgcccgctctcacctgagagctcgcgtgcaggcccagtccaccaatgacggcctggatttccaggagagccagctggtgaagaagctggtggagccacctccacagggatgtcagggatccgtgatcagctttccatctccacgctccggaccaggatctcctgctcagtggctgctgtacacacaccccacccattcctggcagagagctgacctgggagcttatctgaacccccgcccccctgctcctgaggcttggagcgagcctgtgctgctggctaagggctcctgcgcctacagcgacctgcagtctatgggcacaggcccagatggctctcccctgttcggctgtctgtacgaggctaatgactatgaggagatcgtgttcctgatgtttaccctgaagcaggcttttccagccgagtatctgccacagggaggaggaggaggaggcgtgagaccagtgaacatcacagagcccaccctggagctgctgcactcttcctgcgatcccaatgccttccatagcacaatccagctgtactgttttatctatggccacatcctgaacgacgtgagcgtgtcttggctgatggacgatcgcgagatcacagataccctggctcagaccgtgctgatcaaggaggagggcaagctggccagcacatgctctaagctgaatatcaccgagcagcagtggatgtccgagtccaccttcacctgcaaggtgacatctcagggcgtggactacctggctcacaccagacgctgtcctgatcatgagccaaggggcgtgatcacatacctgatcccaccctctccactggacctgtatcagaacggcgcccccaagctgacctgtctggtggtggatctggagtccgagaagaacgtgaatgtgacatggaatcaggagaagaagacctccgtgtccgccagccagtggtacacaaagcaccataacaatgccaccacatccatcaccagcatcctgccagtggtggctaaggactggatcgagggctacggctatcagtgcatcgtggaccatcccgatttccctaagccaatcgtgaggagcatcacaaagacccctggacagcggtctgccccagaggtgtacgtgttccctccacccgaggaggagtctgaggataagcggacactgacctgtctgatccagaacttctttcctgaggacatcagcgtgcagtggctgggcgatggcaagctgatctctaattcccagcactctaccacaaccccactgaagtctaacggctccaatcagggcttctttatcttctccaggctggaggtggctaagacactgtggacccagcggaagcagtttacatgccaggtcatccatgaggccctgcagaagcccagaaagctggagaagacaatcagcacctctctgggcaacacctccctgcgccctagccaccatcaccatcaccattga

SEQ ID NO: 65 - DNA, 2463 bp, Artificial Sequence
  Description of Artificial Sequence Synthetic polynucleotide
atgcctctgctgctgctgctgccactgctgtgggctggcgctctggctatgagacctgcggacctgcccccgcgccccatggaagaatccccggcgtccagctctgccccgacagagacggaggagccggggtccagtgcagaggtcatggaagaagtgacaacatgctccttcaacagccctctgttccggcaggaagatgacagagggattacctaccggatcccagccctgctctacataccccccacccacaccttcctggcctttgcagagaagcgttctacgaggagagatgaggatgctctccacctggtgctgaggcgagggttgaggattgggcagttggtacagtgggggcccctgaagccactgatggaagccacactaccggggcatcggaccatgaacccctgtcctgtatgggagcagaagagtggttgtgtgttcctgttcttcatctgtgtgcggggccatgtcacagagcgtcaacagattgtgtcaggcaggaatgctgcccgcctttgcttcatctacagtcaggatgctggatgttcatggagtgaggtgagggacttgactgaggaggtcattggctcagagctgaagcactgggccacatttgctgtgggcccaggtcatggcatccagctgcagtcagggagactggtcatccctgcgtatacctactacatcccttcctggttcttttgcttccagctaccatgtaaaaccaggcctcattctctgatgatctacagtgatgacctaggggtcacatggcaccatggtagactcattaggcccatggttacagtagaatgtgaagtggcagaggtgactgggagggctggccaccctgtgctatattgcagtgcccggacaccaaacaggtgccgggcagaggcgctcagcactgaccatggtgaaggctttcagagactggccctgagtcgacagctctgtgagcccccacatggttgccaagggagtgtggtaagtttccggcccctggagatcccacataggtgccaggactctagcagcaaagatgcacccaccattcagcagagctctccaggcagttcactgaggctggaggaggaagctggaacaccgtcagaatcatggctcttgtactcacacccaaccagtaggaaacagagggttgacctaggtatctatctcaaccagacccccttggaggctgcctgctggtcccgcccctggatcttgcactgtgggccctgtggctactctgatctggctgctctggaggaggagggcttgtttgggtgtttgtttgaatgtgggaccaagcaagagtgtgagcagattgccttccgcctgtttacacaccgggagatcctgagtcacctgcagggggactgcaccagccctggtaggaacccaagccaattcaaaagcaatggaggaggaggaggaggagtgagaccagtgaacatcacagagcccaccctggagctgctgcactcttcctgcgatcccaatgccttccatagcacaatccagctgtactgttttatctatggccacatcctgaacgacgtgagcgtgtcttggctgatggacgatcgcgagatcacagataccctggctcagaccgtgctgatcaaggaggagggcaagctggccagcacatgctctaagctgaatatcaccgagcagcagtggatgtccgagtccaccttcacctgcaaggtgacatctcagggcgtggactacctggctcacaccagacgctgtcctgatcatgagccaaggggcgtgatcacatacctgatcccaccctctccactggacctgtatcagaacggcgcccccaagctgacctgtctggtggtggatctggagtccgagaagaacgtgaatgtgacatggaatcaggagaagaagacctccgtgtccgccagccagtggtacacaaagcaccataacaatgccaccacatccatcaccagcatcctgccagtggtggctaaggactggatcgagggctacggctatcagtgcatcgtggaccatcccgatttccctaagccaatcgtgaggagcatcacaaagacccctggacagcggtctgccccagaggtgtacgtgttccctccacccgaggaggagtctgaggataagcggacactgacctgtctgatccagaacttctttcctgaggacatcagcgtgcagtggctgggcgatggcaagctgatctctaattcccagcactctaccacaaccccactgaagtctaacggctccaatcagggcttctttatcttctccaggctggaggtggctaagacactgtggacccagcggaagcagtttacatgccaggtcatccatgaggccctgcagaagcccagaaagctggagaagacaatcagcacctctctgggcaacacctccctgcgccctagccaccatcaccatcaccattga

SEQ ID NO: 66 - DNA, 2532 bp, Artificial Sequence
  Description of Artificial Sequence Synthetic polynucleotide
atgcctctgctgctgctgctgccactgctgtgggctggcgctctggctatgggggtccctcgtaccccttcacggacagtgctcttcgagcgggagaggacgggcctgacctaccgcgtgccctcgctgctccccgtgccccccgggcccaccctgctggcctttgtggagcagcggctcagccctgacgactcccacgcccaccgcctggtgctgaggaggggcacgctggccgggggctccgtgcggtggggtgccctgcacgtgctggggacagcagccctggcggagcaccggtccatgaacccctgccctgtgcacgatgctggcacgggcaccgtcttcctcttcttcatcgcggtgctgggccacacgcctgaggccgtgcagatcgccacgggaaggaacgccgcgcgcctctgctgtgtggccagccgtgacgccggcctctcgtggggcagcgcccgggacctcaccgaggaggccatcggtggtgccgtgcaggactgggccacattcgctgtgggtcccggccacggtgtgcagctgccctcaggccgcctgctggtacccgcctacacctaccgcgtggaccgccgagagtgttttggcaagatctgccggaccagccctcactccttcgccttctacagcgatgaccacggccgcacctggcgctgtggaggcctcgtgcccaacctgcgctcaggcgagtgccagctggcagcggtggacggtgggcaggccggcagcttcctctactgcaatgcccggagcccactgggcagccgtgtgcaggcgctcagcactgacgagggcacctccttcctgcccgcagagcgcgtggcttccctgcccgagactgcctggggctgccagggcagcatcgtgggcttcccagcccccgcccccaacaggccacgggatgacagttggtcagtgggccccgggagtcccctccagcctccactcctcggtcctggagtccacgaacccccagaggaggctgctgtagacccccgtggaggccaggtgcctggtgggcccttcagccgtctgcagcctcggggggatggccccaggcagcctggccccaggcctggggtcagtggggatgtggggtcctggaccctggcactccccatgccctttgctgccccgccccagagccccacgtggctgctgtactcccacccagtggggcgcagggctcggctacacatgggtatccgcctgagccagtccccgctggacccgcgcagctggacagagccctgggtgatctacgagggccccagcggctactccgacctggcgtccatcgggccggcccctgaggggggcctggtttttgcctgcctgtacgagagcggggccaggacctcctatgatgagatttccttttgtacattctccctgcgtgaggtcctggagaacgtgcccgccagccccaaaccgcccaaccttggggacaagcctcgggggtgctgctggccctccggaggaggaggaggaggagtgagaccagtgaacatcacagagcccaccctggagctgctgcactcttcctgcgatcccaatgccttccatagcacaatccagctgtactgttttatctatggccacatcctgaacgacgtgagcgtgtcttggctgatggacgatcgcgagatcacagataccctggctcagaccgtgctgatcaaggaggagggcaagctggccagcacatgctctaagctgaatatcaccgagcagcagtggatgtccgagtccaccttcacctgcaaggtgacatctcagggcgtggactacctggctcacaccagacgctgtcctgatcatgagccaaggggcgtgatcacatacctgatcccaccctctccactggacctgtatcagaacggcgcccccaagctgacctgtctggtggtggatctggagtccgagaagaacgtgaatgtgacatggaatcaggagaagaagacctccgtgtccgccagccagtggtacacaaagcaccataacaatgccaccacatccatcaccagcatcctgccagtggtggctaaggactggatcgagggctacggctatcagtgcatcgtggaccatcccgatttccctaagccaatcgtgaggagcatcacaaagacccctggacagcggtctgccccagaggtgtacgtgttccctccacccgaggaggagtctgaggataagcggacactgacctgtctgatccagaacttctttcctgaggacatcagcgtgcagtggctgggcgatggcaagctgatctctaattcccagcactctaccacaaccccactgaagtctaacggctccaatcagggcttctttatcttctccaggctggaggtggctaagacactgtggacccagcggaagcagtttacatgccaggtcatccatgaggccctgcagaagcccagaaagctggagaagacaatcagcacctctctgggcaacacctccctgcgccctagccaccatcaccatcaccattga

SEQ ID NO: 67 - protein, 5 aa, Homo sapiens
TPGTI

SEQ ID NO: 68 - protein, 6 aa, Homo sapiens
TPGTIN

SEQ ID NO: 69 - protein, 7 aa, Homo sapiens
TPGTINI

SEQ ID NO: 70 - protein, 8 aa, Homo sapiens
TPGTINIT

SEQ ID NO: 71 - protein, 9 aa, Homo sapiens
TPGTINITW

SEQ ID NO: 72 - protein, 9 aa, Homo sapiens
GTVNLTWSR

SEQ ID NO: 73 - protein, 6 aa, Artificial Sequence
  Description of Artificial Sequence Synthetic 6xHis tag
HHHHHH